SEQUENCE LISTING (10 sequences)

SEQ ID NO: 1 - DNA, 31 bp, Artificial Sequence
  Synthetic oligonucleotide
tgtaacatcacagcatcaccgccatcagtct

SEQ ID NO: 2 - DNA, 30 bp, Artificial Sequence
  Synthetic oligonucleotide
gactgatggcgcactacgacactacaatgt

SEQ ID NO: 3 - DNA, 31 bp, Artificial Sequence
  Synthetic oligonucleotide
acattgtagtgtcgtagtgcgccatcagtct

SEQ ID NO: 4 - DNA, 41 bp, Artificial Sequence
  Synthetic oligonucleotide
ggattctaatacgactcactatagggatcgcaccagcgtgt

SEQ ID NO: 5 - DNA, 21 bp, Artificial Sequence
  Synthetic oligonucleotide
cgtggatgaggagccgcagtg

SEQ ID NO: 6 - DNA, 21 bp, Artificial Sequence
  Synthetic oligonucleotide
ctgggaatcgcaccagcgtgt

SEQ ID NO: 7 - DNA, 32 bp, Artificial Sequence
  Synthetic oligonucleotide
cgctcagcggccgcagcatcaccgccatcagt

SEQ ID NO: 8 - DNA, 33 bp, Artificial Sequence
  Synthetic oligonucleotide
cgctcagcggccgcgtcgtagtgcgccatcagt

SEQ ID NO: 9 - DNA, 200 bp, Artificial Sequence
  Synthetic oligonucleotide
atcgcaccagcgtgtnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncactgcggctcctca

SEQ ID NO: 10 - DNA, 200 bp, Artificial Sequence
  Synthetic oligonucleotide
tgaggagccgcagtgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnacacgctggtgcgat

(19) United States
(12) Patent Application Publication
(10) Pub. No.: US 2015/0126377 A1
(43) Pub. Date: May 7, 2015

(54) SELECTION OF NUCLEIC ACIDS BY SOLUTION HYBRIDIZATION TO OLIGONUCLEOTIDE BAITS

(71) Applicants: Massachusetts Institute of Technology, Cambridge, MA (US); President and Fellows of Harvard College, Cambridge, MA (US); Whitehead Institute for Biomedical Research, Cambridge, MA (US)

(72) Inventors: ANDREAS GNIRKE, Wellesley, MA (US); Chad Nusbaum, Newton, MA (US); Eric S. Lander, Cambridge, MA (US)

(73) Assignees: Massachusetts Institute of Technology, Cambridge, MA (US); President and Fellows of Harvard College, Cambridge, MA (US); Whitehead Institute for Biomedical Research, Cambridge, MA (US)

(21) Appl. No.: 14/509,497

(22) Filed: Oct. 8, 2014

Related U.S. Application Data
(63) Continuation of application No. 12/365,650, filed on Feb. 4, 2009, now abandoned.
(60) Provisional application No. 61/206,386, filed on Jan. 30, 2009.
(60) Provisional application No. 61/063,489, filed on Feb. 4, 2008.

(51) Int. Cl. C12Q 1/68

(52) U.S. Cl. CPC C12Q 1/6811
USPC 506/2; 506/9

(57) ABSTRACT

Methods of selection of nucleic acids using solution hybridization, methods of sequencing nucleic acids including such selection methods, and products for use in the methods are disclosed.

### RELATED APPLICATIONS

This application is a continuation of U.S. patent application Ser. No. 12/365,650, filed Feb. 4, 2009, and claims the benefit under 35 U.S.C. §119(e) of U.S. provisional application 61/063,489, filed Feb. 4, 2008, and U.S. provisional application 61/206,386, filed Jan. 30, 2009, the entire disclosures of which are incorporated herein by reference.

### GOVERNMENT INTEREST

This work was funded in part by the National Human Genome Research Institute under grant number HG03067-05. The government has certain rights in this invention.

### FIELD OF THE INVENTION

The invention relates to methods of selection of nucleic acids using solution hybridization, methods of sequencing nucleic acids including such selection methods, and products for use in the methods.

### BACKGROUND OF THE INVENTION

Direct selection of nucleic acids for isolation of cDNAs and other nucleic acid molecules was developed in the 1990s. As described by Lovett et al. (Direct selection: A method for the isolation of cDNAs encoded by large genomic regions. Proc. Natl. Acad. Sci. 88:9628-9632, 1991) and Parimoo et al. (cDNA selection: efficient PCR approach for the selection of cDNAs encoded in large chromosomal DNA fragments. Proc. Natl. Acad. Sci. 88: 9623-9627, 1991), direct selection involves hybridization of a library of cDNAs to an immobilized genomic clone. Nonspecific hybrids are eliminated and selected cDNAs are eluted. The selected cDNAs are then amplified and are either cloned or subjected to further selection/amplification cycles. See also: Lovett, Direct selection of cDNAs with large genomic DNA clones. In Molecular Cloning: A Laboratory Manual, Edn. 3, Vol. 2, 2001, (J. Sambrook and D. W. Russell, eds.) Cold Spring Harbor Press, Cold Spring Harbor, N.Y.; Del Mastro and Lovett, Isolation of coding sequences from genomic regions using direct selection. Methods Mol Biol. 68: 183-199, 1997.

More recently, Lovett and coworkers have described a direct selection protocol in which biotinylated bacterial artificial chromosomes (BACs) are used for selection by hybridization with total genomic DNA as a target, followed by amplification of the selected sequences by PCR to isolate DNA from a specific genomic location (Bashiardes et al., Direct genomic selection. Nat. Methods 2(1): 63-69, 2005).

This same approach has been recently used to select genomic sequence corresponding to specific regions in a sample (Albert et al., Direct selection of human genomic loci by microarray hybridization. Nat. Methods. 4(11):903-905, 2007). High-density microarrays were used in a solid-state capture method for the enrichment of specific human genomic sequences for high-throughput sequencing. Microarrays were used to capture individual gene exons, and single long segments corresponding to entire gene loci. The targeted exon-containing segments had a median size of >600 bases which is large compared to the median size of protein-coding exons (120 bp; Clamp et al. Proc. Natl. Acad. Sci. USA 104, 19428-19433, 2007). The long segments were 200 kb, 500 kb, 1 Mb, 2 Mb and 5 Mb and excluded repeat sequences. The direct selection method was described as a substitute for multiplex PCR for the large-scale analysis of genomic regions. The same method using high-density capture microarrays was described by Hodges et al. (Genome-wide in situ exon capture for selective resequencing. Nat. Genetics. 39:1522-1527, 2007) who applied it genome-wide and showed that array capture works best for genomic DNA fragments that are ˜500 bases long, thereby limiting the enrichment and sequencing efficiency for very short dispersed targets such as protein-coding exons.

Porreca et al. described a method of multiplex amplification (Porreca et al., Nature Meth. 4:931-936, 2007). Multiplex amplification uses primer extension to copy, rather than capture, a strand of the targeted genomic DNA. The method utilizes the formation of covalently closed circular molecules which are resistant to digestion with exonuclease while linear side products from mispriming events are eliminated. Circular molecules are then amplified and sequenced. While having a low background of non-targeted sequences, the multiplex amplification method permitted less than 20% of the targets to be detected by deep sequencing of the multiplex amplified material. Moreover, the concentration and hence sequence coverage of the recovered targets was much less uniform than desirable. Finally, allelic drop-out was observed: in many cases only one of the two alleles present in the original DNA samples was found.

### SUMMARY OF THE INVENTION

The techniques described above face key technical challenges in that neither meets all four requirements for a selection method to be truly useful: 1) low unspecific (off-target) and specific (e.g., specifically captured near-target) background of unwanted sequences, 2) experiment-to-experiment and sample-to-sample reproducibility, 3) even representation of sequencing targets and 4) balanced recovery of alleles. By targeting exon-containing segments and array-capturing fragments that are about three times larger than an average exon one obtains much unwanted sequence that also must be sequenced and then separated from the desired sequence information. By multiplex amplifying target exons one obtains less unwanted sequence. However, the highly uneven recovery of targets and poor reproducibility precludes systematic comparative sequencing studies in multiple individual genomes of the same set of targets. Moreover, the allele bias and allele drop-out limits its utility for the study of outbred populations of diploid species such as the human.

All the techniques described above generate enriched genome fractions wherein the selected targets show extreme variation in molarity. Certain targets are recovered at a reduced rate, particularly targets that have extreme base composition. Some targets are not recovered at all. Moreover, the molar variation has not been well characterized in previous studies (Bashiardes et al., 2005). For direct selection techniques to be a practical and economical method for systematic resequencing, it is necessary to ensure that a substantial fraction of the targeted bases are represented and covered by sequence at a level that is equal or greater than a reasonable fraction of the mean coverage (averaged over all target bases), for example, at least half the targeted bases achieving at least half the mean coverage.

It now has been discovered that selection of nucleic acids can be carried out using solution hybridization with oligonucleotide bait sequences. The invention features several unexpected features. First, the selection methods described herein select nucleic acids such that there is an unexpected evenness of sequence coverage in the selected materials; thus, the differences in molarity of different captured sequences are minimized, and are unexpectedly less than is found with previous multiplex amplification or direct selection methods. Second, only a very small amount of nucleic acid sample is needed for successful selection. Third, the length of the bait sequences is unexpectedly important in that baits with >100 bases are more specific and effective capture agents. Fourth, complex mixtures of bait sequences and nucleic acids being directly selected work better than expected. Selection of tens or hundreds of thousands of different nucleic acid sequences is possible, such as a large fraction of the total number of exons of the human genome. Fifth, RNA sequences unexpectedly can be used effectively as bait sequences and even more unexpectedly are at least as good as DNA bait sequences. Sixth, the recovery of the two alleles at heterozygous single-nucleotide polymorphic (SNP) loci is unexpectedly even and shows virtually no allele bias or allele drop-out. Seventh, the experiment-to-experiment reproducibility of target representation in captured sequences is surprisingly high. Eighth, unexpectedly, copy number variations of individual selected targets in the biological samples lead to corresponding copy number variations in the captured target sequences; thus, sequencing hybrid selected targets can not only be used to detect qualitative differences (e.g., single-base changes), but also quantitative differences between nucleic acid samples. Ninth, unexpectedly, bait sequences can also be designed for sequences that represent the cellular RNA and be used to select RNA or cDNA derived from RNA.

Selection as described herein dramatically simplifies large-scale exon resequencing by avoiding the need to amplify hundreds of thousands of exons from each DNA sample. Preliminary experiments have demonstrated that the procedure can be made to work at significant scale using cDNA clones as capture baits. Synthetic baits derived from oligonucleotides that are customized and eluted from microarray chips is a flexible system that can yield relatively uniform coverage across the exon targets. Thus, for example, it is possible to resequence all of the coding exons in a genome using the methods of the invention.

Unlike previous capture methods in solution, the methods of the invention can target any sequence, whether it has been cloned or not, whether it happens to be present in a clone in a reference library or not. Using synthetic bait sequences also allows for targeting of known sequence variants (e.g., common mutations).

The present invention can be applied not only to coding exons in a genome, but to any arbitrarily defined sequenced portion of a genome or even metagenome (i.e., the genomes of all organisms and individuals present in a community of organisms or DNA sample).

The present invention can also be applied to the transcriptome, (i.e. the RNA transcribed and expressed from the genome in a cell, tissue, organ, organism or community of organisms) and to cDNAs derived from the transcriptome.

The present invention in some embodiments combines low cost parallel synthesis of oligonucleotides on chips and intrinsic advantages of solution hybridization, e.g., favorable binding kinetics, higher sensitivity, smaller reaction volumes, and hence less material needed. These features have important implications for cost and sensitivity of targeted sequencing.

The present invention also allows, in some embodiments, the use of a panel of amplification (e.g., PCR) products as bait. For example, a pool of 10,000 specific PCR products amplified from human DNA can be used as template to generate a complex pool of RNA baits for solution hybrid selection.

According to one aspect of the invention, methods for solution-based selection of nucleic acids are provided. The methods include hybridizing in solution (1) a group of nucleic acids and (2) a set of bait sequences, to form a hybridization mixture, contacting the hybridization mixture with a molecule or particle that binds to or is capable of separating the set of bait sequences from the hybridization mixture, and separating the set of bait sequences from the hybridization mixture to isolate a subgroup of nucleic acids that hybridize to the bait sequences from the group of nucleic acids, wherein the subgroup of nucleic acids is a part or all of a set of target sequences that is desired to be selected. The sequence composition of the set of bait sequences determines the nucleic acids directly selected from the group of nucleic acids.

In some embodiments, the set of bait sequences comprises an affinity tag on each bait sequence. Preferably the affinity tag is a biotin molecule or a hapten.

In certain embodiments of the foregoing methods, the molecule or particle that binds to or is capable of separating the set of bait sequences from the hybridization mixture binds to the affinity tag. Preferably the molecule or particle that binds to or is capable of separating the set of bait sequences is an avidin molecule, or an antibody that binds to the hapten or an antigen-binding fragment thereof.

In some embodiments of the foregoing methods, the set of bait sequences is derived from (i.e., produced using) synthetic long oligonucleotides. In some preferred embodiments, the set of bait sequences is derived from (i.e., produced using) oligonucleotides synthesized on a microarray.

In some embodiments of the foregoing methods, the bait sequences are oligonucleotides between about 100 nucleotides and 300 nucleotides in length. Preferably the bait sequences are oligonucleotides between about 130 nucleotides and 230 nucleotides in length. More preferably the bait sequences are oligonucleotides of between about 150 and 200 nucleotides in length. In additional embodiments of the foregoing methods, the bait sequences are oligonucleotides between about 300 nucleotides and 1000 nucleotides in length.

In some embodiments, the target-specific sequences in the oligonucleotides are between about 40 and 1000 nucleotides in length, more preferably between about 70 and 300 nucleotides, more preferably between about 100 and 200 nucleotides, and more preferably still between about 120 and 170 nucleotides in length.

In some preferred embodiments of the foregoing methods, the pool of synthetic oligonucleotides contains forward and reverse complemented sequences for the same target sequence whereby the oligonucleotides with reverse-complemented target specific sequences also carry reverse complemented universal tails. This will lead to RNA transcripts that are the same strand, i.e., not complementary to each other.

In other embodiments of the foregoing methods, the bait sequences are oligonucleotides containing degenerate or mixed bases at one or more positions. In still other embodiments, the bait sequences include multiple or substantially all known sequence variants present in a population of a single species or community of organisms.

In other embodiments of the foregoing methods, the set of bait sequences comprises cDNAs or is derived from cDNAs.

In other embodiments of the foregoing methods, the set of bait sequences comprises pools of amplification products (e.g., PCR products) that are amplified out of genomic DNA, cDNA or cloned DNA.

In some embodiments of the foregoing methods, the set of bait sequences is produced according to methods described hereinbelow. Certain of these methods include obtaining a pool of synthetic long oligonucleotides, originally synthesized on a microarray and amplifying the oligonucleotides to produce a set of bait sequences. In some embodiments, the methods include adding a RNA polymerase promoter sequence at one end of the bait sequences, and synthesizing RNA sequences using RNA polymerase.

In other embodiments of the foregoing methods, the set of bait sequences is produced using known nucleic acid amplification methods, such as PCR. For example, a set of bait sequences (e.g., 10,000 bait sequences) can be specifically amplified using human DNA or pooled human DNA samples as the template, or RNA, according to known methods.

In yet another embodiment, specific subsets of a genome are isolated by physical means (e.g. by flow-sorting of individual chromosomes or by microdissection of cytogenetically and microscopically distinct features of chromosome preparations) followed by specific or non-specific nucleic acid amplification methods that are well known to those skilled in the art.

In some embodiments of the foregoing methods, the bait sequences in the set of bait sequences are RNA molecules. In some embodiments the bait sequences are chemically or enzymatically modified or in vitro transcribed RNA molecules including but not limited to those that are more stable and resistant to RNase.

In other embodiments of the foregoing methods, the group of nucleic acids is fragmented genomic DNA. In some of these embodiments, the group of nucleic acids includes less than 50% of genomic DNA, such as a subfraction of genomic DNA that is a reduced representation or a defined portion of a genome, e.g., that has been subfractionated by other means, while in other of these embodiments the group of nucleic acids comprises all or substantially all genomic DNA.

In certain embodiments of the foregoing methods, the target sequences or subgroup of nucleic acids comprises substantially all exons in a genome. In other embodiments of the foregoing methods, the target sequences or subgroup of nucleic acids comprises exons from selected genes of interest. In some embodiments the selected genes of interest comprise genes involved in a disease, while in other embodiments the selected genes of interest are genes that are not involved in a disease. Such genes may be involved in a biological pathway or process. In still other embodiments, the target sequences or subgroup of nucleic acids comprises a set of cDNAs or viral sequences.

In still other embodiments of the foregoing methods, the group of nucleic acids comprises environmental samples. In such embodiments, the target sequences or subgroup of nucleic acids comprises 16S rRNA or other evolutionary conserved sequences.

In further embodiments of the foregoing methods, the target sequences or subgroup of nucleic acids comprises promoters, enhancers, 5′ untranslated regions, 3′ untranslated regions, transposon exclusion zones, and/or a set of distinct genomic features, which set constitutes less than 10% of a genome. In some embodiments, the set constitutes less than 1% of a genome.

In some embodiments, the target sequences or subgroup of nucleic acids comprises one or more large genomic regions, that span less than 1 Mb, more than 1 Mb, more than 5 Mb, more than 20 Mb, more than 100 Mb, or more than 500 Mb of the genome. In some embodiments, the targets correspond to chromosomes, subchromosomal regions or regions containing cytogenetically defined chromosomal aberrations such as translocations or supernumerary marker chromosomes.

In still other embodiments, the target sequences or subgroup of nucleic acids comprises more than 10%, more than 50% or essentially all the genome, for example for applications that include but are not limited to enriching the DNA of one species within a DNA sample that contains the DNA from other species.

In certain preferred embodiments, sequences that are not unique, or similar to other sequences, or repetitive or low complexity, are excluded from the pool of capture baits.

In certain embodiments of the foregoing methods, the number of bait sequences in the set of bait sequences is less than 1,000. In other embodiments, the number of bait sequences in the set of bait sequences is greater than 1,000, greater than 5,000, greater than 10,000, greater than 20,000, greater than 50,000, greater than 100,000, or greater than 500,000.

In some embodiments of the foregoing methods, the group of nucleic acids comprises less than 5 micrograms of nucleic acids. Preferably the group of nucleic acids comprises less than 1 microgram of nucleic acids.

In some embodiments, the group of nucleic acids is amplified by whole-genome amplification methods such as random-primed strand-displacement amplification.

In preferred embodiments of the foregoing methods, the group of nucleic acids is fragmented by physical or enzymatic methods and ligated to synthetic adapters, size-selected (e.g., by preparative gel electrophoresis) and amplified (e.g., by PCR).

In other preferred embodiments, the fragmented and adapter-ligated group of nucleic acids is used without explicit size selection or amplification prior to hybrid selection.

In preferred embodiments, the selected subgroup of nucleic acids (“catch”) is amplified (e.g., by PCR) before being analyzed by sequencing or other methods. In other embodiments, the selected subgroup of nucleic acids is analyzed without such an amplification step.

In some embodiments of the foregoing methods, the methods further include subjecting the isolated subgroup of nucleic acids to one or more additional rounds of solution hybridization with the set of bait sequences.

In other embodiments of the foregoing methods, the method further includes subjecting the isolated subgroup of nucleic acids to one or more additional rounds of solution hybridization with a different set of bait sequences.

In still other embodiments of the foregoing methods, the group of nucleic acids consists of RNA or cDNA derived from RNA. In some embodiments, the RNA consists of total cellular RNA. In other embodiments, certain abundant RNA sequences (e.g., ribosomal RNAs) have been depleted. In some preferred embodiments, the poly(A)-tailed mRNA fraction in the total RNA preparation has been enriched. In some preferred embodiments, the cDNA is produced by random-primed cDNA synthesis methods. In other preferred embodiments, the cDNA synthesis is initiated at the poly(A) tail of mature mRNAs by priming by oligo(dT)-containing oligonucleotides. Methods for depletion, poly(A) enrichment, and cDNA synthesis are well known to those skilled in the art.

In additional embodiments of the foregoing methods, the molarity of at least 50% of the isolated subgroup of nucleic acids is within 20-fold of the mean molarity. More preferably, the molarity of at least 75% of the isolated subgroup of nucleic acids is within 10-fold of the mean molarity. Even more preferably, the molarity of at least 75% or the isolated subgroup of nucleic acids is within 3-fold of the mean molarity.

In some embodiments of the foregoing methods, at least 50% of the bases in the isolated subgroup of nucleic acids are present at and can therefore achieve sequence coverage with at least 50% of the mean averaged over all target bases. In preferred embodiments, 75% or more of the targeted bases comprise and can achieve at least 50% of the mean. For example, see FIG. 9 which shows >60% for exon capture and ˜80% for regional capture.

In some embodiments of the foregoing methods, the method is carried out using automated or semi-automated liquid handling.

According to another aspect of the invention, methods of sequencing or resequencing nucleic acids are provided. The methods include isolating by solution hybridization a subgroup of nucleic acids according to the methods described herein, and subjecting the isolated subgroup of nucleic acids to nucleic acid sequencing.

According to another aspect of the invention, methods for genotyping nucleic acids are provided. The methods include isolating by solution hybridization a subgroup of nucleic acids according to the methods described herein, and subjecting the isolated subgroup of nucleic acids to genotyping.

According to still another aspect of the invention, methods of producing a set of bait sequences are provided. The methods include obtaining a pool of synthetic long oligonucleotides, originally synthesized on a microarray and amplifying the oligonucleotides to produce a set of bait sequences.

In some embodiments of the foregoing methods, the oligonucleotides are amplified by polymerase chain reaction (PCR). In some of these embodiments, the amplified oligonucleotides are reamplified by rolling circle amplification or hyperbranched rolling circle amplification. The same methods also can be used to produce bait sequences using human DNA or pooled human DNA samples as the template. The same methods can also be used to produce bait sequences using subfractions of a genome obtained by other methods, including but not limited to restriction digestion, pulsed-field gel electrophoresis, flow-sorting, CsCl density gradient centrifugation, selective kinetic reassociation, microdissection of chromosome preparations and other fractionation methods known to those skilled in the art.

In some embodiments of the foregoing methods, the methods further include size selecting the amplified oligonucleotides.

In other embodiments of the foregoing methods, the methods further include reamplifying the oligonucleotides using one or more biotinylated primers. Preferably the reamplification process is PCR.

In some embodiments of the foregoing methods, the oligonucleotides comprise universal sequences at the end of each oligonucleotide attached to the microarray, and the methods further include removing the universal sequences from the oligonucleotides. Preferably such methods also include removing the complementary strand of the oligonucleotides, annealing the oligonucleotides, and extending the oligonucleotides. In some of these embodiments, the methods for reamplifying the oligonucleotides use one or more biotinylated primers. Preferably the reamplification process is PCR. The methods of these embodiments also can include size selecting the amplified oligonucleotides.

In some embodiments of the foregoing methods, the oligonucleotides are between about 100 nucleotides and 300 nucleotides in length. Preferably the oligonucleotides are between about 130 nucleotides and 230 nucleotides in length. More preferably the oligonucleotides are between about 150 and 200 nucleotides in length.

In some embodiments the target-specific sequences in the oligonucleotides for selection of exons and other short targets are between about 40 and 1000 nucleotides in length, more preferably between about 70 and 300 nucleotides, more preferably between about 100 and 200 nucleotides, and more preferably still between about 120 and 170 nucleotides in length.

According to another aspect of the invention, methods of producing a set of RNA bait sequences are provided. The methods include producing a set of bait sequences according to the methods described herein, adding a RNA polymerase promoter sequence at one end of the bait sequences, and synthesizing RNA sequences using RNA polymerase.

In some embodiments of the foregoing methods, the RNA polymerase is a T7 RNA polymerase, a SP6 RNA polymerase or a T3 RNA polymerase.

In other embodiments of the foregoing methods, the RNA polymerase promoter sequence is added at the ends of the bait sequences by reamplifying the bait sequences. Preferably the reamplifying is performed by PCR.

In embodiments where the bait sequences are amplified by PCR with specific primer pairs out of genomic or cDNA, adding an RNA promoter sequence to the 5′ end of one of the two specific primers in each pair will lead to a PCR product that can be transcribed into a RNA bait using standard methods.

According to yet another aspect of the invention one or more sets of bait sequences are provided that are produced according to any of the methods described herein.

According to still another aspect of the invention, methods for determining the presence or sequence of a nucleic acid sequence, cell, tissue or organism in a sample are provided. The methods include obtaining a sample containing nucleic acids, subjecting the nucleic acids in the sample to solution-based selection of nucleic acids according to any of the methods described herein or sequencing according to the methods described herein or genotyping according to the methods described herein, and determining the presence or sequence of one or more nucleic acids of the subgroup of nucleic acids obtained by selection. The presence or sequence of the one or more nucleic acids indicates the presence of a nucleic acid sequence, cell, tissue or organism in the sample.

In some embodiments of the foregoing methods, the nucleic acid sequence, cell, tissue or organism is a bacterial cell, a tumor cell or tissue, a virus, or a nucleic acid mutation. In some embodiments, the nucleic acid mutation is a germ line mutation or a somatic mutation.

In some embodiments of the foregoing methods, the sample containing nucleic acids is an environmental sample.

These and other aspects of the invention, as well as various embodiments thereof, will become more apparent in reference to the drawings and detailed description of the invention.

### BRIEF DESCRIPTION OF THE DRAWINGS

FIG. 1 schematically shows an exemplary selection process of an embodiment of the invention. As shown, bait sequences are hybridized in solution with a group of nucleic acids (the “pond”). The hybridized sequences are then captured using a moiety linked to or incorporated in the bait sequences. The hybrid-selected targets represent a subgroup of the starting group of sequences (“pond”), and referred to here as the “catch”. This subgroup of sequences can then be subjected to sequencing.

FIG. 2 schematically shows and describes two basic exemplary processes to obtain bait sequences from microarray chips. On the left side of FIG. 2, an embodiment of bait sequences is described in which each bait sequence is produced from a single oligonucleotide. In this embodiment, the oligonucleotide includes universal bases at each end (A, B) and x target-specific bases between the universal sequences. On the right side of FIG. 2, an embodiment of bait sequences is described in which a longer bait sequence is produced from two oligonucleotides. In this embodiment, the oligonucleotide includes universal bases at each end (A,B on one oligonucleotide and B,C on the second oligonucleotide) and x target-specific bases between the universal sequences. The two oligonucleotides anneal via n target specific bases.

FIG. 3 schematically shows preferred embodiments of methods for producing single-stranded bait sequences from single oligonucleotides (e.g., as described on the left side of FIG. 2), including the production of biotinylated RNA bait sequences by transcription using biotinylated ribonucleotides after the addition of a T7 RNA polymerase promoter sequence (“T7”) and biotinylated DNA bait sequences by denaturation of double stranded DNA molecules after addition of biotin moieties. The biotin moieties are represented by solid circles attached to the bait sequences.

FIG. 4 schematically shows preferred embodiments of methods for producing longer bait sequences from two oligonucleotides (e.g., as described on the right side of FIG. 2) by overlap extension. Subsequent production of biotinylated RNA bait sequences and biotinylated DNA bait sequences proceeds as described above for FIG. 3.

FIG. 5 schematically shows a preferred embodiment of producing single-stranded non-self-complementary RNA bait sequences from synthetic oligonucleotides that represent different strands of the double-stranded DNA target. Two reverse complementary oligonucleotide sequences are designed such that the entire sequences (including the universal tails) are reverse complementary to each other. One of them contains a poly(G) stretch (indicated in red) that may be more difficult to synthesize chemically than the corresponding poly(C) stretch (green) on the complementary oligonucleotide. Both oligonucleotides give rise to the very same double-stranded PCR product and hence to the same RNA strand. If the synthetic oligodeoxynucleotide containing the poly(G) stretch is absent or under-represented and the reverse complementary poly(C) containing oligo present at the normal concentration the net effect of the deleterious poly(G) sequence would be a 50% reduction of the biotinylated RNA bait for the corresponding target. If the reverse-complemented oligodeoxynucleotide had not been present, the bait for this target would be completely absent. If both sequences are synthesized at equal amounts, reverse-complementary oligodeoxynucleotides may anneal to each other. However, the final single-stranded biotinylated RNA bait is the same strand, regardless which strand has been chemically synthesized initially. This method provides some redundancy at the chemical synthesis stage without interfering with the goal of producing a pool of non-self-complementary single-stranded RNA baits that can drive the solution hybridization more efficiently than a mixture containing reverse complementary RNA molecules that can anneal to each other.

FIG. 6 schematically shows three exemplary methods for sequence coverage of short isolated target sequences (e.g., exons) by short-read sequencing and the sequence coverage of target sequences obtained therefrom. FIG. 6A shows end-sequenced target sequences with short (e.g., 36 base) reads. FIG. 6B shows short-read (e.g., 36 base) shotgun-sequenced target sequences following concatenation and shearing. FIG. 6C shows short-read (e.g., 36-base) end-sequencing of fragments that have been hybrid selected using staggered baits. The graphs in lower portions of FIG. 6A, FIG. 6B and FIG. 6C show the sequence coverage of a target using each of the sequencing methods. The Y axis of the plots represents the number of sequencing reads at each base along the sequencing target. Fragments that overlap only partially with the bait (and therefore end near the middle) form less stable hybrids and are therefore under-represented. End sequencing with short reads (A) gives rise to high sequence coverage near and beyond the end of the capture baits and a pronounced dip in the middle. Concatenating, re-shearing and shotgun sequencing (B) improves coverage in the middle and increases the fraction of sequenced bases that are on bait and on target. An overlapping set of staggered baits gives rise to relatively even coverage along the target by mere end sequencing the catch with short reads, obviating the need for concatenating and re-shearing but requiring substantially more oligonucleotide baits per target (C). Staggering the baits widens the genome segment that is covered by bait, and therefore widens the impact zone and reduces the fraction of specifically caught sequence that is on-target.

FIG. 7 schematically shows a preferred method for end-sequencing short targets (e.g., exons). Shown are cumulative coverage profiles that sum the per-base sequencing coverage along free-standing single-bait targets that demonstrate the effects of increasing the read length of end sequences. End sequencing with short (e.g., 36 base) reads (FIG. 7A) produced a bimodal profile with high sequence coverage near and slightly beyond the ends of the baits (indicated by the horizontal blue bar). End sequencing with longer (e.g., 76 base) reads (FIG. 7B) produces a larger fraction of bases that are on bait and on target. This preferred method obviates the need for shot-gun library construction while avoiding the dip in coverage seen with very-short end sequencing (A) or the widening of the bait-covered segment when using staggered baits (as in FIG. 6C).

FIG. 8 shows the sequence coverage along the non-repetitive fraction of a larger genomic target that was selected by the method disclosed in the present invention. Sequence corresponding to bait is marked in blue. Segments that had more than 40 repeat-masked bases per 170-base window were not targeted by baits and received little or no coverage with sequencing reads aligning uniquely to the genome.

FIG. 9 shows what fraction of the targeted bases achieve a given normalized sequence coverage. The fraction of target bases is plotted on the Y axis. The X axis is the observed depth of sequence coverage divided by the mean sequence coverage averaged over all target bases. An ideal hypothetical hybrid selection with completely even coverage across all targets would result in a horizontal line connecting X,Y coordinates (0,1) and (1,1) and then dropping vertically to (1,0). An actual hybrid selection using 22,000 200mer oligos targeting>15,000 exons as bait resulted in the plot in FIG. 9A which shows that more than 60% of the target bases received 50% or more of the mean coverage. Almost 80% of the target bases received ⅕ of the mean coverage. FIG. 9B is a similar plot for a regional capture experiment targeting the non-repetitive fraction (0.75 Mb) of four genomic regions spanning 1.7 Mb in total. The curve in FIG. 9B is flatter than the curve in FIG. 9A, indicating more uniform representation of sequencing targets in the regional catch, where 80% of the targeted bases achieved at least half the mean coverage and 86% of the targeted bases had ⅕ of the mean coverage.

FIG. 10 demonstrates the reproducibility of hybrid selection performed by the method of the present invention. For each target exon (n=15,565), the ratio of the mean coverage in two independent hybrid selection experiments performed on the same source DNA (NA15510) was plotted over its mean coverage in one experiment (FIG. 10A). Coverage was normalized to adjust for the different number of sequencing reads. The average ratio (black line) is close to 1. Standard deviations are indicated by purple lines. The graph on the right (FIG. 10B) shows base-by-base sequence coverage along one target in three independent hybrid selections, two of them performed on NA15510 (purple and teal lines) and one on NA11994 source DNA (black). Note the similarities at this fine resolution of the three profiles which were normalized to the same height. The position of the exemplary target exon and bait is indicated by red and blue bars, respectively.

FIG. 11 shows the unexpected quantitative response to copy number variations of hybrid selection. Sequence coverage observed in hybrid-selected DNA from one sample was averaged over each target and plotted of the coverage observed in the targets selected from another sample. Targets that have no variation in copy number between the two samples scatter around the diagonal. Targets that are over-represented in one sample are significantly above or below the diagonal indicated by the black line. In FIG. 11A, target coverage in a female sample was plotted over target coverage in a male sample. Targets on chromosome X (red dots that cluster mainly within the elliptical area) are present twice in females and only once in males and are therefore above the diagonal. FIG. 11B compares coverage of targets in a tumor (Y-axis) vs. a normal sample (X-axis). Target exons for two genes A and B that were known to be amplified in this tumor are indicated by red and green dots, respectively, and cluster mainly within the two ellipses. The slope of the data points for genes A and B indicate gene-amplification levels in the tumor of ˜40-fold and ˜9-fold, respectively.

FIG. 12 shows an example of a laboratory set-up that allows the semiautomated processing of up to 96 hybrid selections in parallel. The exemplary apparatus shown consists of a peristaltic pump wash station with 96 individual chimneys that washes tips and disposes of waste (top row left), a I/O controlled Heat Block set at the temperature (e.g., 65° C.) for the high-stringency wash (top row center), a station for 165 μl sterile aerosol filtered tips that perform liquid handling steps throughout the bead-capture process (top row right), a 96-well plate containing 0.1N NaOH for the final elution of the catch off the beads (middle row left), a six-bar 96-well magnet plate that holds magnetic beads to the sides of wells so supernatant can be aspirated and discarded (middle row center), a position to hold the 96-well hybridization plate containing the solution hybrid selection reaction mixes (middle row right), a second I/O controlled heat block (bottom row left) to preheat high-stringency wash buffer in a deep-well block to the appropriate temperature (e.g., 65° C.), a position for a 96-well plate holding low-stringency wash buffer (bottom row center), and a position at the lower row right that holds a 96-well plate containing magnetic beads to be added to the hybridization plate above until the protocol prompts the user to exchange this plate for a 96-well plate containing 1M Tris-HCl neutralization buffer to receive the supernatant of the alkaline catch elution step off the beads and held at 4° C. until the end of the run.

FIG. 13 shows additional normalized coverage distribution plots for exon captures. Shown is the fraction of targeted exon bases in the human genome achieving coverage equal or greater than the normalized coverage indicated on the X-axis. The hybrid-selected exon catch was either concatenated, re-sheared and shotgun sequenced with 36-base Illumina GA-I reads (a, b) or directly end sequenced with 76-base Illumina GA-II reads (c, d). To show the tail end of the distributions (b, d), the normalized coverage on the X-axis was truncated at 5. The absolute per base coverage was divided by the mean coverage which was 21 for shotgun (a, b) and 94 for long-read end sequences (c, d). Note that these graphs show the normalized coverage of targeted exon bases proper whereas FIG. 3a in the main text shows the normalized coverage for bait sequence.

FIG. 14 shows extended normalized coverage distribution plot for regional capture. To show the tail end of the coverage distribution the normalized coverage on the x-axis was truncated at 5 instead of at 1. Shown is the fraction of bait-covered bases in the human genome achieving coverage equal or greater than the normalized coverage indicated on the X-axis. The hybrid-selected regional catch was concatenated, sheared and shot-gun sequenced with 36-base Illumina GA-I reads. The absolute per base coverage was divided by the mean coverage which was 221 in this particular experiment.

FIG. 15 shows effects of GC content. Normalized coverage-distribution plots for exon-bait sequence broken down by GC content of the baits (shown on the right). Only about 20-30% of bases in extremely GC-rich (70-80%) bait sequences achieved half the mean coverage whereas ˜80% of bases in baits with 50-60% GC achieved this coverage.

FIG. 16 shows sample-to-sample consistency of targeted sequencing. Tumor and normal control DNA samples from a single individual were amplified by random-primed whole-genome strand-displacement amplification before they were converted to “pond” libraries for fishing with a bait that targeted 3,739 exons. The PCR-amplified catches were concatenated, sheared and shotgun Illumina sequenced with 36-base reads. Top: For each exon, the ratio of the mean sequence coverage of tumor to normal DNA was plotted over its mean coverage in normal DNA. Coverage was normalized to adjust for the different number of sequencing reads. The average ratio (blue line) is close to 1. Bottom: Base-by-base sequence coverage along one target exon in tumor (red) and normal (blue) DNA. The blue horizontal bars and shaded areas indicate the position of the two baits for the target exon.

### DETAILED DESCRIPTION OF THE INVENTION

We reasoned that selection of nucleic acids using solution hybridization might be especially useful for isolating targets such as the protein-coding regions of the genome. The ideal bait would consist of individual DNA fragments containing each exon of interest, together with just enough surrounding sequence to ensure strong hybridization. Moreover, the ideal protocol would ensure relatively equimolar output of each target.

As proof of principle, we used cDNAs as baits. These baits had the advantages of being “off the shelf” and of requiring only one bait per gene. However, they have the disadvantage that some exons are too small to allow efficient capture. Below, we describe a protocol to avoid this problem.

In our initial experiments, we used bait consisting of 35 full-length human cDNAs containing ˜400 exons. Baits were biotinylated by nick translation. We sheared total human DNA, ligated to adapters for PCR amplification and hybridized it to the biotinylated bait. Samples were washed under standard high stringency wash conditions (0.1×SSC, 65° C.). We sequenced the resulting product using the 454 platform (454 Life Sciences, part of Roche Applied Science, Branford, Conn.). After one round of hybridization, 19% of the products mapped to the immediate vicinity of one of the ˜400 exons (4000-fold average enrichment). After two rounds, the proportion rose to 74% (15,000-fold average enrichment), providing an average of 200× coverage of the target in a single 454 sequencer run. Thus, tremendous enrichment for the desired targets was obtained.

We next characterized the variation in coverage. We found that 95% of all exons were represented and 78% were present at a molarity within 20-fold of the mean. The exons that were poorly covered were mostly short or GC-poor. Exon coverage was strongly correlated with DNA melting temperature.

With the aim of achieving more uniform recovery of fragments, we explored changes to the wash conditions. The reagent tetramethylammonium chloride (TMACl) is known to reduce the differential hybridization strength of A:T and C:G base pairs. By using 3 M TMACl at 55° C., we improved the uniformity of recovery, and were able to improve the enrichment of GC-poor exons by a factor of four.

Encouraged by this success, we scaled up the experiment to ˜800 cDNAs containing >7000 exons. We found that the vast majority of reads (60%) mapped to exons, confirming that we had achieved substantial enrichment. The variation in coverage was similar to the previous experiments. This experiment demonstrates implementation of selection that works reasonably well with a complex mixture of baits.

An improved protocol for exon selection was then designed. Before extending the protocol to the entire human gene set, we aimed to further reduce the variation in coverage and to increase the proportion of exons that are selected. Our analysis showed that exons of size>140 bp worked relatively well, whereas small exons were sometimes problematic. We reasoned that a good solution would be to replace the cDNA baits with individual genomic DNA fragments of ˜200 bp, corresponding to each exon (with flanking sequence).

We tested this solution by generating such exon-specific baits, using PCR of genomic DNA. As hoped, we found that using genomic baits greatly increased the efficiency of capturing small exons. On average, the exons that were poorly captured with cDNAs showed no systematic underrepresentation with exon-specific genomic baits.

We then focused on generating a large collection of exon-specific baits covering as many as ˜15,000 exons, a sizeable fraction of the ˜200,000 exons in the human genome. In accordance with one preferred embodiment, we obtained the desired 200-base bait sequences as a custom pool of synthetic oligonucleotides originally synthesized as an oligonucleotide array. The oligonucleotides can be liberated from the array by chemical cleavage followed by removal of the protection groups. Each oligonucleotide contains 170 target-specific bases and 15 base universal tails on each end. For another embodiment, pools of 22,000 oligonucleotides of length 170 bases are generated. Two 170-base oligonucleotides for each target are designed, overlapping by ˜30 bases and containing an appropriate tail for PCR amplification on each end. After enzymatic cleavage of one of the tails, and degradation of one of the strands, the single-stranded products can be hybridized, made fully double stranded by filling in, and amplified by PCR. In this manner, it is possible to produce bait molecules that contain >300 contiguous target-specific bases which is more than can be chemically synthesized. Such long baits are useful for applications that require very high specificity and sensitivity, or for applications that do not necessarily benefit from limiting the length of the bait molecules (capture of long contiguous genomic regions, for example).

In some embodiments, oligonucleotides from microarray chips are tested for efficacy of hybridization, and a production round of microarray chips ordered on which oligonucleotides are grouped by their capture efficacy, thus compensating for variation in bait efficacy. For large projects, oligonucleotide pools can be aggregated to form a relatively small number of composite pools, such that there is little variation in capture efficacy among them.

The oligonucleotides from the chips are synthesized once, and then can be amplified to create a set of oligonucleotides that can be used many times. This approach generates a universal reagent that can be used as bait for a large number of selection experiments, thereby amortizing the chip cost to be a small fraction of the sequencing cost. Alternatively, bait sequences can be produced using known nucleic acid amplification methods, such as PCR, using human DNA or pooled human DNA samples as the template. Moreover, the coverage of each target can be assessed and targets that yield similar coverage can be grouped. Distinct sets of bait sequences can be created for each group of targets, further improving the representation.

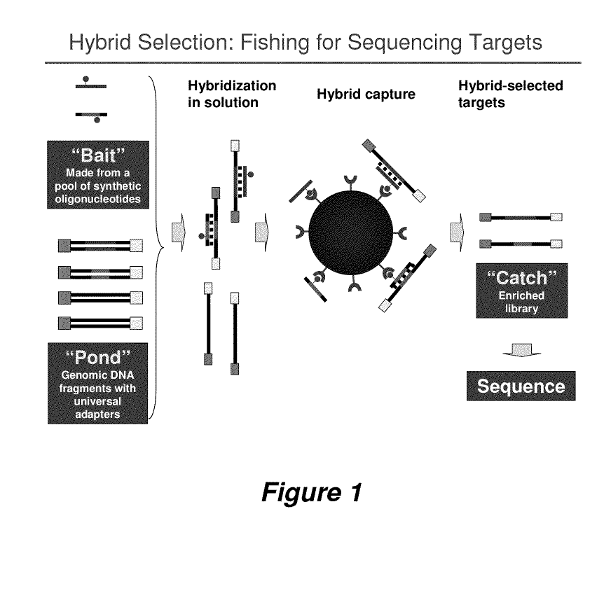

The invention provides methods for solution-based selection of nucleic acids. The methods include hybridizing in solution (1) a group of nucleic acids from which nucleic acids are to be directly selected and (2) a set of bait sequences, to form a hybridization mixture. See FIG. 1 for a schematic representation of one embodiment of the method. The hybridization mixture is contacted with a molecule or particle that binds to or is capable of separating the set of bait sequences from the hybridization mixture, and then the set of bait sequences is separated from the hybridization mixture to isolate from the group of nucleic acids a subgroup of nucleic acids that hybridize to the bait sequences. The sequence composition of the set of bait sequences determines the nucleic acids directly selected from the group of nucleic acids.

The selection methods of the invention are carried out by hybridization in solution, i.e., neither the oligonucleotide bait sequences nor the group of nucleic acids (containing target nucleic acid molecules that are desired to be selected from the group of nucleic acids) being selected from are attached to a solid surface. Performing the selection method by hybridization in solution minimizes the reaction volume and therefore the amount of target nucleic acid necessary to achieve the concentration necessary to drive the hybridization reaction. Performing the selection method described herein using hybridization in solution also means that amplification of the nucleic acids is not required. The ability to select without amplification is important for applications that are not compatible with amplification. For example, bisulfite sequencing for methylation analysis is not compatible with amplification because amplification replaces 5-methyl cytosine in the genomic DNA with cytosine, or vice versa. This ability also eliminates amplification bias during the preparation of the hybridization-ready group of nucleic acids.

Performance of the methods of the invention does not require bulky and expensive equipment (e.g., in contrast to solid-phase hybridization methods, which use chip-specific washing stations etc.) and has therefore better long-term potential for processing many more samples in parallel (e.g., in 96-well plate format).

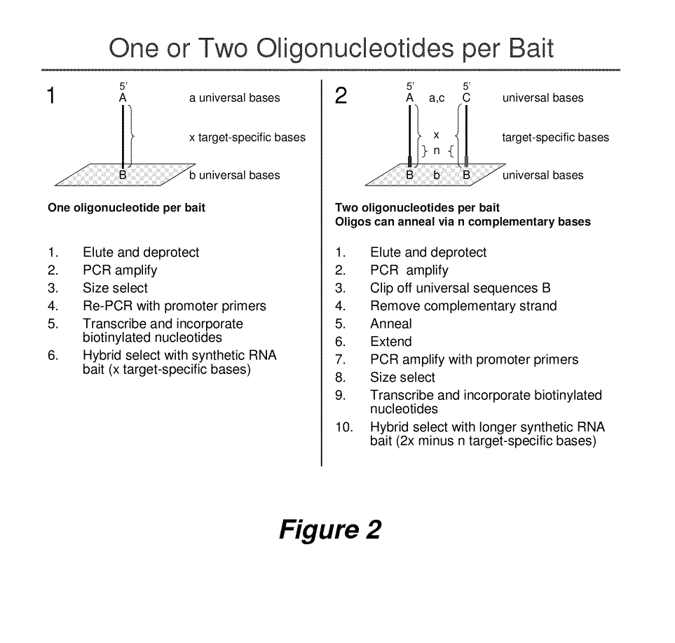

The methods of the invention in some embodiments use long synthetic oligonucleotides including the bait sequences, which in one embodiment are about 200 bases in length, of which 170 bases are target-specific “bait sequence”. The other 30 bases (15 on each end) are universal arbitrary tails used for PCR amplification. The tails can be any sequence selected by the user. In other embodiments, the bait sequence oligonucleotides are between about 150-200 nucleotides in length. In other embodiments, the set of bait sequences is produced using known nucleic acid amplification methods, such as PCR, e.g., using human DNA or pooled human DNA samples as the template. As used herein, the term “bait sequence” can refer to the target-specific bait sequence or the entire oligonucleotide including the target-specific “bait sequence” and other nucleotides of the oligonucleotide. See the left panel of FIG. 2 for a schematic of exemplary oligonucleotides having a bait sequence, and a description of an exemplary method of making and using the oligonucleotides in the methods of the invention.

See also the Examples below for a description of exemplary methods of production and use of the oligonucleotides bait sequences in the selection methods of the invention. In this embodiment, oligonucleotides of 200 bases are used without the need to combine two oligonucleotides to form a single bait sequence. The oligonucleotides are converted to biotinylated RNA bait sequences as described in the Examples. The subgroup of nucleic acids that is selected using the bait sequences is concatenated and sheared as is described elsewhere herein, but also can be end sequenced.

Long oligonucleotides minimize the number of oligonucleotides necessary to capture the target sequences (for example, in one example of the methods of the invention 22,000 oligonucleotides were used for ˜15,000 exons; i.e. in many cases 1 oligonucleotide per exon. The mean length of the protein-coding exons in the human genome is 164 bp; the median length is 120 bp; ˜75% of the ˜300,000 known protein-coding exons are 170 bp or shorter (Clamp et al., 2007).

Longer baits are more specific and capture better than shorter ones. As a result, the success rate per oligonucleotide bait sequence is higher than with short oligonucleotides. This has important implications for capturing exon-sized targets: the preferred minimum bait-covered sequence is the size of one bait (e.g., 120-170 bases). In determining the length of the bait sequences, one also can take into consideration that unnecessarily long baits catch more unwanted DNA directly adjacent to the target.

Another selection methodology (Albert et al., 2007) tiles oligonucleotides across a much wider window (typically 600 bases). This method also captures DNA fragments that are much larger (˜500 bases) than a typical exon. As a result, much more unwanted flanking non-target sequences are selected.

Another advantage of long oligonucleotide baits over shorter ones is that the former are more tolerant to polymorphisms in the targeted region in the DNA samples. The bait sequences are typically—although not necessarily—derived from a reference genome sequence. If the target sequence in the actual DNA sample deviates from the reference sequence, for example if it contains a SNP, it will hybridize less efficiently to the bait and may therefore be under-represented or, in the worst case, completely absent in the sequences hybridized to the bait sequences. Allelic drop-outs due to SNPs are less likely with the longer synthetic baits molecules described in this invention for the reason that a single mispair in, e.g., 120-170 bases will have much less of an effect on hybrid stability than a single mismatch in, 20 or 70 bases, which are the typical bait or primer lengths in multiplex amplification and microarray capture, respectively.

Typically bait sequences are designed from reference sequences, such that the baits are optimal for catching targets of the reference sequences. However, in some embodiments, bait sequences are designed using a mixed base (i.e., degeneracy). For example, the mixed base(s) can be included in the bait sequence at the position(s) of a common SNP or mutation, to optimize the bait sequences to catch both alleles (i.e., SNP and non-SNP; mutant and non-mutant). The same approach may be used for other target sequences such as phylogenetically conserved sequences in viruses or 16S rRNA sequences in environmental samples: use of degenerate base(s) at non-conserved position(s) permit selecting sequences that deviate from a reference sequence. In other embodiments, all known sequence variations (or a subset thereof) can be targeted with multiple oligonucleotide baits, rather than by using mixed degenerate oligonucleotides.

Applications of the foregoing methods include using a library of oligonucleotides containing all known sequence variants (or a subset thereof) of a particular bacterial gene or genes for metagenomic sequencing of this particular gene or genes in environmental or medical specimens. Additional applications include analyzing functional classes of genes or whole or partial pathways of genes. For example, rather than trying to isolate and analyze by sequencing a single gene in a metagenome to make inferences about the presence of absence of particular species, genus or families of species in a sample, one can prepare a phylogenetically diverse capture bait for all genes known or suspected to be involved in a particular biological process or pathway, for example amino acid metabolism, and use this bait to isolate and analyze by sequencing all genes relevant to this process in a bacterial metagenome to make functional inferences about the presence, absence of the genetic potential to carry out certain biochemical reactions in the environment or sample of interest.

Further applications include enriching and analyzing a whole taxonomic class of organisms. These applications include, for example, using a library of oligonucleotides containing sequences and sequence variants of a particular taxonomic class of bacteria to allow deep metagenomic sequencing of this particular group of bacteria, which may represent only a small percentage of the bacteria in these samples and would otherwise be difficult or costly to sequence at great depth. For example, one can design and synthesize baits that are specific to archaeal genomes which may not be very abundant in certain environments and would therefore be difficult to sample with whole-microbiome sequence-based approaches that do not enrich for low-abundant taxae.

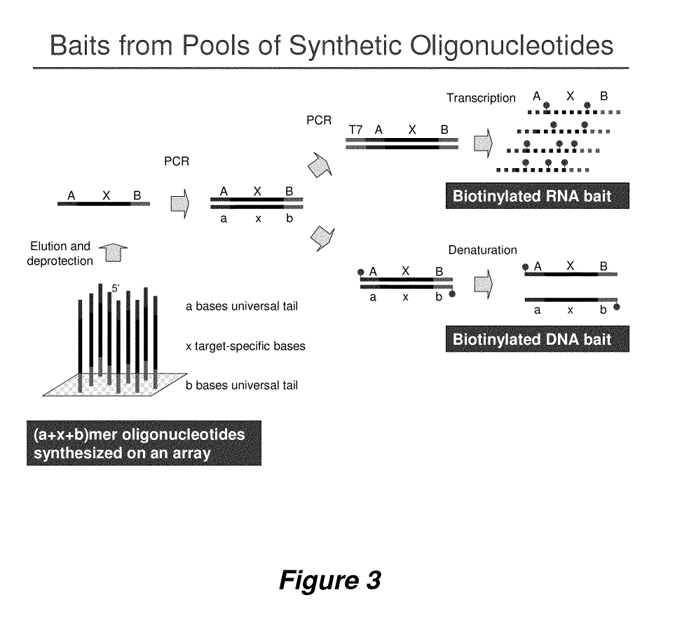

In preferred aspects of the invention, the bait sequences include an affinity tag and more preferably there is an affinity tag on each on each bait sequence in a set of bait sequences. Affinity tags include biotin molecules, magnetic particles, haptens, or other tag molecules that permit isolation of molecules tagged with the tag molecule. Such molecules and methods of attaching them to nucleic acids (e.g., the bait sequences used in the methods disclosed herein) are well known in the art. Exemplary methods for making biotinylated DNA and RNA bait oligonucleotides are shown in FIG. 3.

Also known in the art are molecules, particles or devices that bind to or are capable of separating the set of tagged bait sequences from the hybridization mixture. In some embodiments of the methods, the molecules, particles or devices bind to the affinity tag. The molecules, particles or devices in some preferred embodiments is an avidin molecule, a magnet, or an antibody or antigen-binding fragment thereof.

The bait sequences in some embodiments are synthetic long oligonucleotides or are derived from (e.g., produced using) synthetic long oligonucleotides. In certain embodiments, the set of bait sequences is derived from oligonucleotides synthesized in a microarray and cleaved and eluted from the microarray. Exemplary methods are shown and described in FIGS. 2-5. In other embodiments, the bait sequences are produced by nucleic acid amplification methods, e.g., using human DNA or pooled human DNA samples as the template.

Bait sequences preferably are oligonucleotides between about 70 nucleotides and 1000 nucleotides in length, more preferably between about 100 nucleotides and 300 nucleotides in length, more preferably between about 130 nucleotides and 230 nucleotides in length and more preferably still are between about 150 nucleotides and 200 nucleotides in length. Intermediate lengths in addition to those mentioned above also can be used in the methods of the invention, such as oligonucleotides of about 70, 80, 90, 100, 110, 120, 130, 150, 160, 180, 190, 210, 220, 230, 240, 250, 300, 400, 500, 600, 700, 800, and 900 nucleotides in length, as well as oligonucleotides of lengths between the above-mentioned lengths. For selection of exons and other short targets, preferred bait sequence lengths are oligonucleotides of about 100 to about 300 nucleotides, more preferably about 130 to about 230 nucleotides, and still more preferably about 150 to about 200 nucleotides. The target-specific sequences in the oligonucleotides for selection of exons and other short targets are between about 40 and 1000 nucleotides in length, more preferably between about 70 and 300 nucleotides, more preferably between about 100 and 200 nucleotides, and more preferably still between about 120 and 170 nucleotides in length. For selection of targets that are long compared to the length of the capture baits, such as genomic regions, preferred bait sequence lengths are typically in the same size range as the baits for short targets mentioned above, except that there is no need to limit the maximum size of bait sequences for the sole purpose of minimizing targeting of adjacent sequences.

In certain embodiments, bait sequences contain all sequences in the regions or targets of interest. In preferred embodiments, the bait sequences exclude certain sequences that are non-unique or repetitive in the genome. In preferred embodiments of hybrid selection in mammalian genomes such as the human genome, each bait contains less than 40 bases that are flagged as repetitive and/or low-complexity by algorithms and computer programs well known to those skilled in the art. In one preferred embodiment, the bait sequences are laid onto the reference sequence followed by removal of certain baits that contain less than the pre-defined limit of bases that are flagged as repetitive or low-complexity in whole-genome annotations. The baits can be laid onto the reference genome sequence such that neighboring baits overlap, such that there are no gaps or overlaps between adjacent baits, or such that there are gaps.

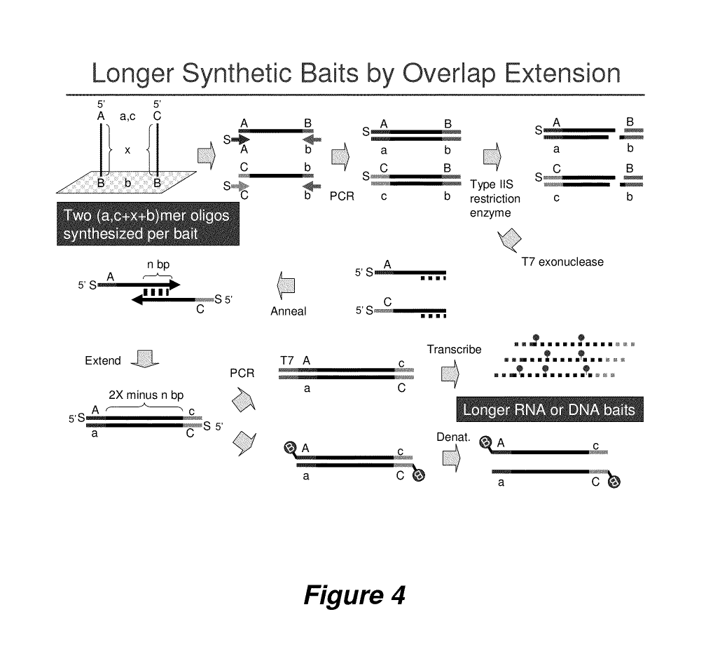

Methods to prepare longer oligonucleotides for bait sequences are well known in the art. One preferred method for preparing longer oligonucleotides by overlap extension from shorter oligonucleotides eluted from an array is shown schematically and described in FIGS. 2 and 4. One such method shown schematically in FIG. 4 includes removing the complementary strand of the oligonucleotides, pairwise annealing of the oligonucleotides via complementary sequence (“n” target-specific nucleotides anneal, see also FIG. 2), and then extending the oligonucleotides. Even longer synthetic oligonucleotide baits can be made by an iterative process whereby the products of the first overlap extension reaction would be further extended either by pairwise annealing among themselves or by annealing to suitable sequences from a separate pool of synthetic oligonucleotides.

In embodiments in which the bait sequences are produced by nucleic acid amplification methods, longer baits can be produced by selecting primer sequences that are spaced apart on the template in a way that produces longer oligonucleotides.

In some embodiments, the bait sequences in the set of bait sequences are RNA molecules. These can be made as described elsewhere herein, using methods known in the art, including de novo chemical synthesis and transcription of DNA molecules using a DNA-dependent RNA polymerase. The RNA molecules can be RNase-resistant RNA molecules, which can be made, for example, by using modified nucleotides during transcription to produce RNA molecules that resist RNase degradation. In preferred embodiments, RNA bait sequences include an affinity tag. In some embodiments, RNA bait sequences are made by in vitro transcription, for example, using biotinylated UTP. Examples of this are shown schematically in FIGS. 3 and 4. In other embodiments, RNA bait sequences are produced without biotin and then biotin is crosslinked to the RNA molecules using methods well known in the art, such as psoralen crosslinking.

As used herein, “group of nucleic acids” means nucleic acids that contain target sequences and are hybridized to bait sequences to select the target sequences. As used herein, “target sequences” are the set of sequences that one desires to isolate from the group of nucleic acids. The term target describes the scope or purpose of the experiment. To use the embodiment of exons as an example, the target sequences can be a specific group of exons, e.g., 500 particular exons. The target sequences, in a different example, can be all ˜300,000 protein-coding exons in the human genome. The sequences that are actually selected from the group of nucleic acids is referred to herein as a “subgroup of nucleic acids”. The term subgroup describes the performance of the method, i.e., that not all of the target sequences are recovered by any particular use of the processes described herein. For example, the subgroup may in some embodiments be a percentage of the target sequences that is as low as 10% or as high as 90%.

The subgroup of nucleic acids, while ideally containing 100% of the target sequences (i.e., when the selection method selects all of the target sequences from the group of nucleic acids) and no additional non-targeted sequences, typically contains less than all of the target sequences and contains some amount of background of unwanted sequences. For example, more typically the subgroup of nucleic acids is at least about 20%, 30%, 40%, 50%, 60%, 70%, 75%, 80%, 85%, 90%, 95%, 98%, 99% or more of the target sequences. The purity of the subgroup (percentage of reads that align to the targets) is typically at least about 20%, 30%, 40%, 50%, 60%, 70%, 75%, 80%, 85%, 90%, 95%, 98%, 99% or more.

The group of nucleic acids in some embodiments is fragmented genomic DNA. Genomic DNA may be fragmented by physical shearing methods, enzymatic cleavage methods, chemical cleavage methods, and other methods well known to those skilled in the art. The group of nucleic acids typically contains all or substantially all of the complexity of the genome. The term “substantially all” in this context refers to the possibility that there may in practice be some unwanted loss of genome complexity during the initial steps of the procedure. However, the methods described herein also are useful in cases where the group of nucleic acids is a portion of the genome, i.e., where the complexity of the genome is reduced by design. In such embodiments, the practitioner may use any selected portion of the genome with the methods described herein.

The target sequences (and the subgroup of nucleic acids) obtained from genomic DNA can include a small fraction of the total genomic DNA, such that it includes less than about 0.0001%, at least about 0.0001%, at least about 0.001%, at least about 0.01% or 0.1% of genomic DNA, or a more significant fraction of the total genomic DNA, such that it includes at least: about 2% of genomic DNA, about 3% of genomic DNA, about 4% of genomic DNA, about 5% of genomic DNA, about 6% of genomic DNA, about 7% of genomic DNA, about 8% of genomic DNA, about 9% of genomic DNA, about 10% of genomic DNA, or more than 10% of genomic DNA.

In some embodiments, the target sequences may include more than 10%, more than 20%, more than 50% or essentially all of the genome. Such embodiments may be used to select targets from a complex mixture of genomes or a metagenome. Examples of applications of such embodiments include but are not limited to the selection of the DNA from one species from a sample containing the DNA from other species. In such applications, the target may include less than 0.0001%, at least 0.0001%, at least about 0.001%, at least about 0.01% or 0.1% of the total complexity of the nucleic acid sequence or metagenome, or a more significant fraction such that it includes at least about 1%, about 2%, about 5%, about 10% or more than 10% of the total complexity of nucleic acid sequences present in the complex sample or metagenome.

In a particular embodiment, the target sequences (and the subgroup of nucleic acids) selected by the solution hybridization selection method of the invention is the set of all exons in a genome. However, the target sequences (and the subgroup of nucleic acids) can include only a portion of exons in a genome, such as greater than 0.1% of genomic exons, greater than 1% of genomic exons, greater than 10% of genomic exons, greater than 20% of genomic exons, greater than 30% of genomic exons, greater than 40% of genomic exons, greater than 50% of genomic exons, greater than 60% of genomic exons, greater than 70% of genomic exons, greater than 80% of genomic exons, greater than 90% of genomic exons, or greater than 95% of genomic exons.

Alternatively, the target sequences and subgroup of nucleic acids can contain exons or other parts of selected genes of interest. The use of specific bait sequences allows the practitioner to select target sequences (ideal set of sequences selected) and subgroups of nucleic acids (actual set of sequences selected) containing as many or as few exons (or other sequences) from a group of nucleic acids as are preferred for a particular selection.

Similarly, the target sequences and subgroup of nucleic acids can include a set of cDNAs. Capturing cDNAs may be used, for example, to analyze the transcriptome, to find splice variants, to identify fusion transcripts (e.g., from genomic DNA translocations), and to obtain evidence to the structure of hypothetical genes. In some embodiments, the analysis of the transcriptome is used to find single base changes and other sequence changes expressed in the RNA fraction of a cell, tissue, organ or organism.

The foregoing exons, cDNAs and other sequences of the group of nucleic acids, target sequences and/or subgroup of nucleic acids can be related or unrelated as desired. For example, selected target sequences and subgroup(s) of nucleic acids may be obtained from a group of nucleic acids that are genes involved in a disease, such as a group of genes implicated in one or more diseases such as cancers, a group of nucleic acids containing specific SNPs, a group of nucleic acids in environmental samples, etc. Other groups of nucleic acids from which target sequences and subgroup(s) of nucleic acids may be selected using the methods of the invention include promoters, enhancers, 5′ untranslated regions, 3′ untranslated regions, transposon exclusion zones, or any set of distinct genomic features, that constitutes less than 10% of a genome. The 10% is by no means a technical limitation of the invention nor should it be construed as one. In certain cases, particularly for, but not limited to, smaller genomes, it may be useful and cost effective to select and analyze more than 10% of a genome. For applications that select sequences from complex DNA samples or metagenomes, the set of distinct genomic features may often constitute more than 10% of a genome, in some case entire genomes or more than one genome. The methods of the invention permit the practitioner to design the set of bait sequences to enable selection of essentially any desired target sequences and subgroup(s) of nucleic acids from the group of nucleic acids.

A variety of samples can be the source of the nucleic acids for selection. For example, the group of nucleic acids can be a part of or isolated from environmental samples, patient samples, such as blood samples or biopsies, archival samples, etc. Such clinical and environmental sequences can be analyzed for a group of viral sequences, a group of bacterial samples, a group of pathogen sequences, etc.

As noted above, one of the unexpected features of the methods of the invention are that solution-based selection can be performed using an unexpectedly small amount of nucleic acids. Thus, in some embodiments, the group of nucleic acids comprises less than 5 micrograms of nucleic acids. More preferably, the group of nucleic acids comprises less than 4, less than 3, less than 2, less than 1, less than 0.8, less than 0.7, less than 0.6, or less than 0.5 micrograms of nucleic acids.

The ability to use small amounts of nucleic acids in the methods is particularly useful because the amount of source DNA often is limiting (even after whole-genome amplification). One protocol that has been tested uses 500 ng of a group of nucleic acids per hybridization with bait sequences. To prepare 500 ng of hybridization-ready nucleic acids (“pond” DNA), one typically begins with 3 μg of genomic DNA. One can start with less, however, if one amplifies the genomic DNA (e.g., using PCR) before the step of solution hybridization. Thus it is possible, but not essential, to amplify the genomic DNA before solution hybridization. There are exceptions, however, where genomic DNA cannot be amplified before solution hybridization, such as in methylation analysis.

It also is unexpected that a large number of bait sequences can be used effectively in solution hybridization. As compared to the earlier direct selection methods that used large bait molecules such as BAC or YAC, it is entirely unexpected that a complex mixture of several thousand bait sequences can effectively hybridize to complementary nucleic acids in a group of nucleic acids and that such hybridized nucleic acids (the subgroup of nucleic acids) can be effectively separated and recovered. Thus it is possible in some embodiments to use a set of bait sequences containing more than 5,000 bait sequences, more than 6,000 bait sequences, more than 7,000 bait sequences, more than 8,000 bait sequences, more than 9,000 bait sequences, more than 10,000 bait sequences, more than 11,000 bait sequences, more than 12,000 bait sequences, more than 13,000 bait sequences, more than 14,000 bait sequences, more than 15,000 bait sequences, more than 16,000 bait sequences, more than 17,000 bait sequences, more than 18,000 bait sequences, more than 19,000 bait sequences, more than 20,000 bait sequences, more than 30,000 bait sequences more than 40,000 bait sequences more than 50,000 bait sequences more than 60,000 bait sequences more than 70,000 bait sequences more than 80,000 bait sequences more than 90,000 bait sequences, more than 100,000 bait sequences, or more than 500,000 bait sequences.

In some instances it may be advantageous to repeat the selection process on the selected subgroup of nucleic acids in order to increase the enrichment of selected nucleic acids. As noted above, after one round of hybridization, a several thousand fold enrichment of nucleic acids was observed. After a second round, the enrichment rose dramatically (e.g., ˜15,000-fold average enrichment for the example cited above), which provided hundreds-fold coverage of the target in a single sequencer run. Thus, for experiments that require enrichment factors not achievable in a single round of hybrid selection, the methods preferentially include subjecting the isolated subgroup of nucleic acids (i.e., a portion or all of the target sequences) to one or more additional rounds of solution hybridization with the set of bait sequences.

Sequential hybrid selection with two different bait sequences (bait 1, bait 2) can be used to isolate and sequence the “intersection”, i.e., the subgroup of DNA sequences that binds to bait 1 and to bait 2. This embodiment can be used for applications that include but are not limited to enriching for interchromosomal or interspecies chimeric sequences. For example, selection of DNA from a tumor sample with a bait specific for sequences on chromosome 1 followed by selection from the product of the first selection of sequences that hybridize to a bait specific for chromosome 2 may enrich for sequences at chromosomal translocation junctions that contain sequences from both chromosomes. Similar experiments are conceivable to enrich for sequences that contain DNA from two species to detect instances of interspecies gene transfer for example. Another use of two sequential selections is to subdivide a phage-display library. This is conceptually similar to the sequential hybrid selection method for isolating and sequencing the intersection described above, except that one would first select for a protein function (for example, enrich phage that display proteins that bind to a fragrant substance) and then select for a DNA function by hybrid selection (for example, to obtain only the subset of phage that contain DNA sequences that are similar to olfactory genes).

Another unexpected feature of the selection methods of the invention is that the molarity of the selected subgroup of nucleic acids can be controlled such that the molarity of any particular nucleic acid is within a small variation of the average molarity of all selected nucleic acids in the subgroup of nucleic acids. Methods for controlling and optimizing the evenness of target representation include but are not limited to rational design of bait sequences based on physicochemical as well as empirical rules of probe design well known in the art, and pools of baits where sequences known or suspected to underperform are overrepresented to compensate for their intrinsic weaknesses. For example, in some embodiments, at least 50% of the isolated subgroup of nucleic acids is within 20-fold of the mean molarity, more preferably within 10-fold of the mean molarity. More preferably, at least 60%, 65%, 70%, 75%, 80%, 85%, 90% or 95% of the isolated subgroup of nucleic acids is within 20-fold of the mean molarity, more preferably within 10-fold of the mean molarity, and more preferably still within 3-fold of the mean molarity.

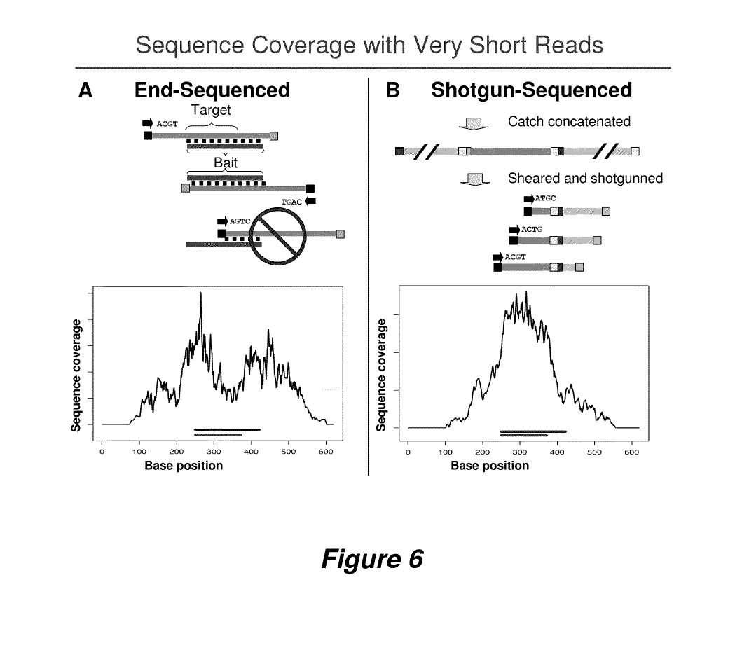

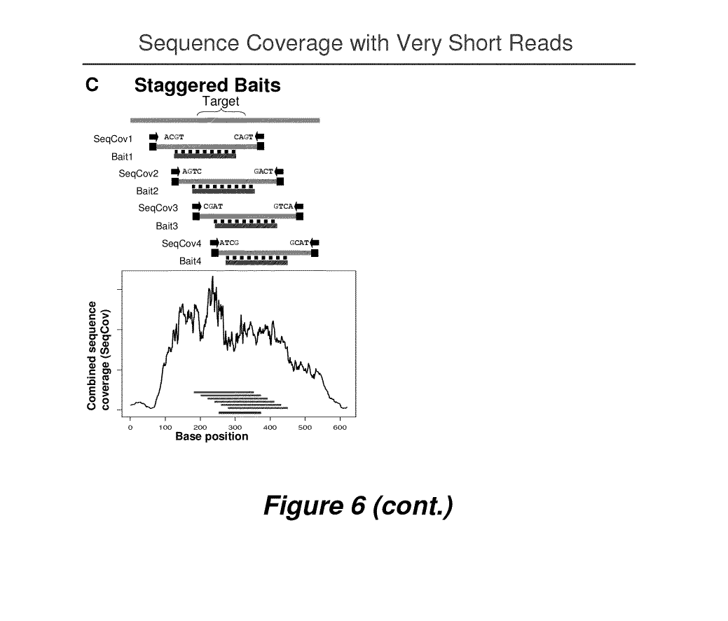

A different way of expressing this unexpected feature of the invention is that the coverage of the target sequences is remarkably even, as is shown in FIG. 6. For example, using the methods of the invention, the percent of target bases having at least 50% of the expected coverage is about 60% for short targets such as protein-coding exons and about 80% for targets that are long compared to the length of the capture baits, such as genomic regions.

The methods of the invention are adaptable to standard liquid handling methods and devices. Thus, in some embodiments, the method is carried out using automated liquid handling technology as is known in the art, such as devices that handle multiwell plates. This can include automated “pond” library construction, and steps of solution hybridization including set-up and post-solution hybridization washes.

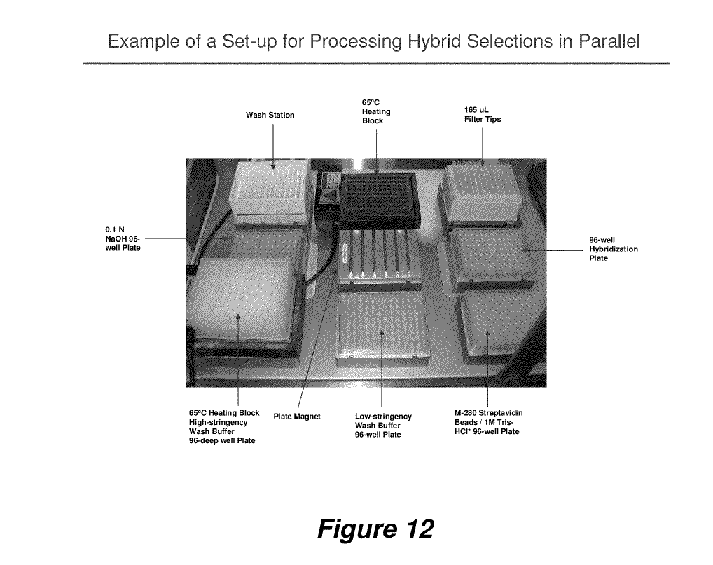

An example of an apparatus that can be used for carrying out such automated methods for the bead-capture and washing steps after the solution hybridization reaction is shown in FIG. 12. The exemplary apparatus is designed to process up to 96 hybrid selections from the bead-capture step through the catch neutralization step in parallel. The minimum set up for an exemplary preferred embodiment of the current invention has a position for a multi-well plate containing streptavidin-coated magnetic beads, a position for the multiwall plate containing the solution hybrid-selection reactions, I/O controlled heat blocks to preheat reagents and to carry out washing steps at a user-defined temperature, a position for a rack of pipet tips, a position with magnets laid out in certain configurations that facilitate separation of supernatants from magnet-immobilized beads, a washing station that washes pipet tips and disposed of waste, and positions for other solutions and reagents such as low and high-stringency washing buffers or the solution for alkaline elution of the final catch. In the example shown, one position has a dual function, and the user is prompted by the protocol to exchange one plate for another.

As those skilled in the art of laboratory automation will appreciate, other steps in preferred methods disclosed here including but not limited to preparation of hybridization baits, the preparation of the group of nucleic acids to be subjected to hybrid selection, setting up and incubating the reaction mixes for the solution hybrid selection, cleaning up the subgroup of selected nucleic acids, amplification steps (e.g., by PCR), size-selection or size exclusion steps whether they are carried out by electrophoresis, chromatography, size-sensitive adsorption or elution methods can also be performed on commercially available or custom devices designed to specifications that are well known to those skilled in the art. In one preferred embodiment of the invention, one or more consecutive handling steps are performed on an individual dedicated apparatus, with manual transfer of reaction plates from one dedicated apparatus to another. In other preferred embodiments, robotic arms, plate hotel and other equipment well known to those in the art can be used to automate longer series of reaction steps, replenish reagents and labware and allow unsupervised processing of multiple sets of nucleic acid samples to be selected with one or more set of capture baits in serial or parallel fashion.

The invention also includes methods of sequencing or resequencing nucleic acids. In these methods, subgroup(s) of nucleic acids are isolated by selection using the methods described herein, i.e., using solution hybridization, and then the isolated subgroup of nucleic acids is subjected to nucleic acid sequencing.

Any method of sequencing known in the art can be used. Sequencing of nucleic acids isolated by the selection methods of the invention preferably is carried out using massively parallel short-read sequencing (e.g., the Solexa sequencer, Illumina Inc., San Diego, Calif.), because the read out generates more bases of sequence per sequencing unit than other sequencing methods that generate fewer but longer reads. However, sequencing also can be carried out using other methods or machines, such as the sequencers provided by 454 Life Sciences (Branford, Conn.), Applied Biosystems (Foster City, Calif.; SOLiD sequencer) or Helicos BioSciences Corporation (Cambridge, Mass.), or by standard Sanger dideoxy terminator sequencing methods and devices.

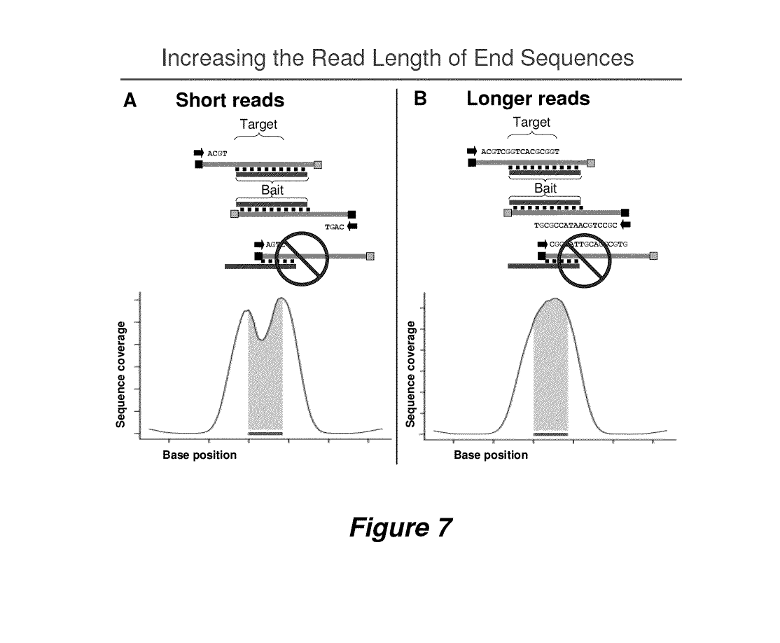

For certain sequencing methods, the directly selected nucleic acids are concatenated and sheared, which is done to overcome the limitations of short sequencing reads. In one embodiment of the invention, each exon-sized sequencing target is captured with a single bait molecule that is about the same size as the target and has endpoints near the endpoints of the target. Only hybrids that form double strand molecules having approximately 100 or more contiguous base pairs survive stringent post-hybridization washes. As a result, the selected subgroup of nucleic acids (i.e., the “catch”) is enriched for randomly sheared genomic DNA fragments whose ends are near the ends of the bait molecules. Mere end-sequencing of the “catch” with very short sequencing reads therefore gives higher coverage near the end (or even outside) of the target and lower coverage near the middle (see FIG. 6A and FIG. 7A).

Concatenating “catch” molecules by ligation and followed by random shearing and shotgun sequencing is one method to get sequence coverage along the entire length of the target sequence (see FIG. 6B). This method produces higher percentage of sequenced bases that are on target (as opposed to near target) than end sequencing with very short reads. Methods for concatenating molecules by co-ligation are well known in the art. Concatenation can be performed by simple blunt end ligation. “Sticky” ends for efficient ligation can be produced by a variety of methods including PCR amplification of the “catch” with PCR primers that have restriction sites near their 5′ ends followed by digestion with the corresponding restriction enzyme (e.g., NotI) or by strategies similar to those commonly used for ligation-independent cloning of PCR products such as partial “chew-back” by T4 DNA polymerase (Aslanidis and de Jong, Nucleic Acids Res. 18:6069-6074, 1990) or treatment of uracil-containing PCR products with UDG glycosylase and lyase endo VIII (e.g., New England Biolabs cat. E5500S).

In another embodiment of the invention, a staggered set of bait molecules is used to target a region, obtaining frequent bait ends throughout the target region. In this embodiment, merely end-sequenced “catch” (i.e., without concatenation and shearing) provides fairly uniform sequence coverage along the entire region that is covered by bait (FIG. 6C) including the actual sequencing target (e.g., an exon). As staggering the bait molecules widens the segment covered by bait, the sequenced bases are distributed over a wider area. As a result, the ratio of sequence on target to near target is lower than for selections with non-overlapping baits that, in many cases, require only a single bait per target.

In another embodiment, end sequencing with slightly longer reads (e.g., 76 bases) is the preferred method for sequencing short selected targets (e.g., exons). Unlike end sequencing with very short reads, this method leads to an unimodal coverage profile without a dip in coverage in the middle (see FIG. 7B). This method is easier to perform than the concatenate and shear method described above, results in relatively even coverage along the targets, and generates a high percentage of sequenced bases fall on bait and on target proper.

In some embodiments, the selected subgroup of nucleic acids will be amplified (e.g., by PCR) prior to being analyzed by sequencing or genotyping. In other embodiments (for example applications where the selected subgroup is analyzed by sensitive analytical methods that can read single molecules), the subgroup can be analyzed without such an amplification step.

The methods of solution hybridization also provide for additional uses, such as using hybrid-selected DNA for DNA assays other than sequencing. For example, one can enrich Plasmodium DNA (or only the DNA segments that contain SNP markers) from DNA prepared from malaria patients for genotyping. The presence of human DNA seems to interfere with genotyping the plasmodium, hence the genotyping methods may work better if the plasmodium DNA is hybrid-selected prior to analysis. This same approach could be used for analysis of other parasites and infectious nucleic acids such as bacteria, fungi, DNA viruses, etc. It also could be used for forensic applications.

The methods of solution hybrid selection also provides for uses where the group of nucleic acids consists of nucleic acids and other biological or chemical constituents (e.g., proteins) and where the hybrid-selected material is subjected to analysis of these non-nucleic acid moieties, or in some cases, of both nucleic acid and non-nucleic acids constituents. Examples include but are not limited to selecting, by solution hybridization via specific nucleic-acid nucleic acid interaction, nucleic acid-protein complexes of interest from a complex mixture prepared from a biological sample followed by mass-spectrometric identification of proteins attached to or co-selected with the selected subgroup of nucleic acids. Analysis of the subgroup of nucleic acids by sequencing or genotyping can be used to measure the specificity of the selection, or, in some cases, to obtain additional information about the nature of the selected subgroup of nucleic acids.

The invention also includes methods for producing a set of bait sequences. The methods include providing or obtaining a nucleic acid array (e.g., microarray chip) that contains a set of synthetic long oligonucleotides, and removing the oligonucleotides from the microarray (e.g., by cleavage or elution) to produce a set of bait sequences. See FIGS. 2-5 for schematic representations of exemplary oligonucleotides and exemplary methods for making pools of bait sequences, including longer bait sequences from two oligonucleotides. Synthesis of oligonucleotides in an array format (e.g., chip) permits synthesis of a large number of sequences simultaneously, thereby providing a set of bait sequences for the methods of selection. The array synthesis also has the advantages of being customizable and capable of producing long oligonucleotides.

In other embodiments, the set of bait sequences is produced using known nucleic acid amplification methods, such as PCR, or other amplification methods described herein or known to the skilled person. For example, a set of bait sequences (e.g., 10,000 bait sequences) can be specifically amplified using human DNA or pooled human DNA samples as the template, according to known methods, whereby spacing of the primers on the template sequence will dictate the length of the resulting oligonucleotide baits.

In some embodiments, the oligonucleotides include universal sequence(s) at the end of each oligonucleotide produced in the microarray. For example, see FIG. 2, in which the universal sequences are designated A, B, and C. The universal sequences can include sequences for amplification (A, B, C). In one embodiment, the target-specific portion of the oligonucleotides contain sequences of length n for annealing two oligonucleotides together for extension (sequence n, see FIG. 2 and FIG. 4).

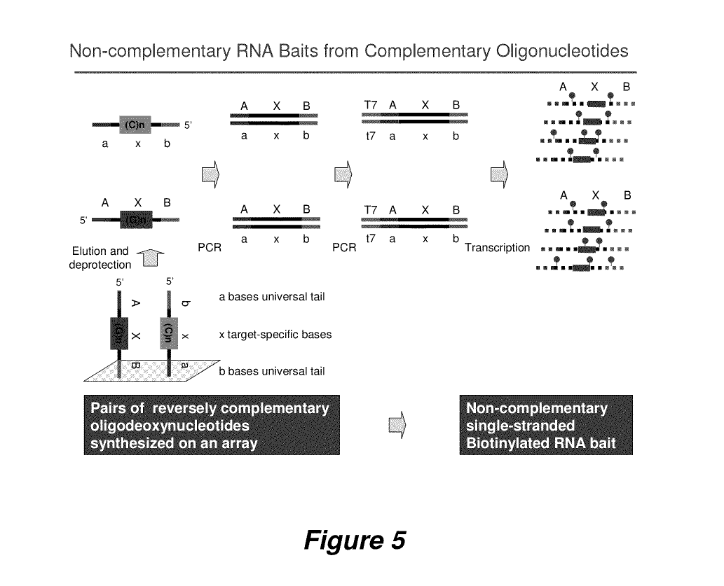

In some preferred embodiments, two reverse complementary oligonucleotides are synthesized on the same microarray. This method provides some redundancy at the chemical synthesis stage while the PCR product and the single-stranded RNA bait transcribed thereof are the same for the two reverse complements. (See FIG. 5). It is well known in the art, that certain sequences (e.g., poly(G) tracks) are refractory standard chemical oligosynthesis chemistry. Synthesizing a reverse complementary “minus” oligonucleotide (containing a less problematic poly(C) track) may produce a functional RNA bait of the same sequence, in cases where the “plus” sequence may fail.

Preferably the methods also include amplifying the oligonucleotides, once removed from the array by elution, to produce a set of bait sequences (see FIGS. 2-5). The synthesized oligonucleotides can be used many times, even thousands of times, and represent an (almost) inexhaustible source of bait sequences. Amplification can be performed using any method of amplification known in the art, such as polymerase chain reaction (PCR). The PCR with primers specific to the universal tails at the end of the synthetic oligonucleotides (see FIGS. 2-5) will also enrich for full-length products of the chemical synthesis as many incomplete truncated products will lack the universal tail at the 5′-end and will therefore not amplify exponentially.

While PCR amplification is preferred to amplify the oligonucleotides, other amplification methods, including other methods that utilizing PCR plus rolling circle amplification can be used.

As an optional step, the amplified oligonucleotides can be selected by size to eliminate short unwanted by-products using standard, well known methods such as gel electrophoresis or HPLC.

It is preferred that the bait sequences be tagged with an affinity tag. As noted above, preferably there is an affinity tag on each on each bait sequence in a set of bait sequences. Affinity tags include biotin molecules, magnetic particles, haptens, or other tag molecules that permit isolation of molecules tagged with the tag molecule. To incorporate a biotin molecule as an affinity tag, for example, the bait oligonucleotides can be reamplified using one or more biotinylated primers in a reamplification process such as PCR. Examples of this are shown schematically in FIGS. 3 and 4.

As noted above, in some embodiments, the oligonucleotides are between about 70 nucleotides and 1000 nucleotides in length, more preferably between about 100 nucleotides and 300 nucleotides in length, more preferably between about 130 nucleotides and 230 nucleotides in length and more preferably still are between about 150 nucleotides and 200 nucleotides in length. In some embodiments the target-specific sequences in the oligonucleotides are between about 40 and 1000 nucleotides in length, more preferably between about 70 and 300 nucleotides, more preferably between about 100 and 200 nucleotides, and more preferably still between about 120 and 170 nucleotides in length. Intermediate lengths in addition to those mentioned above also can be used in the methods of the invention, such as oligonucleotides of about 70, 80, 90, 100, 110, 120, 130, 150, 160, 180, 190, 210, 220, 230, 240, 250, 300, 400, 500, 600, 700, 800, and 900 nucleotides in length, as well as oligonucleotides of lengths between the above-mentioned lengths. For selection of relatively short target sequences such as exons, preferred bait sequence lengths are about 100 to about 300 nucleotides, more preferably about 130 to about 230 nucleotides, still more preferably about 150 to about 200 nucleotides in length. For selection of targets that are long compared to the length of individual bait molecules, such as genomic regions, bait lengths are in the same size range as the baits for short targets mentioned above, except that there is no need to limit the maximum length of bait sequences for the sole purpose of minimizing targeting of adjacent sequences.

RNA molecules preferably are used as bait sequences. A RNA-DNA duplex is more stable than a DNA-DNA duplex, and therefore provides for potentially better capture of nucleic acids. RNA bait sequences can be synthesized using any method known in the art. In some embodiments, in vitro transcription is used, for example based on adding RNA polymerase promoter sequences to one end of oligonucleotides (see FIGS. 3-5 for examples of this embodiment). As is well known in the art, RNA promoter sequences can also be introduced during PCR amplification of bait sequences out of genomic DNA by tailing one primer of each target-specific primer pairs with an RNA-promoter sequence. If RNA is synthesized using biotinylated UTP, single stranded biotin-labeled RNA bait molecules are produced. In preferred embodiments, the RNA baits correspond to only one strand of the double-stranded DNA target. As those skilled in the art will appreciate, such RNA baits are not self-complementary and are therefore more effective as hybridization drivers. In certain embodiments, RNase-resistant RNA molecules are synthesized. Such molecules and their synthesis is well known in the art.

Thus, the invention provides methods of producing a set of RNA bait sequences in which a set of bait sequences is produced as described above, an RNA polymerase promoter sequence at the end(s) of the bait sequences, and the RNA bait sequences are synthesized using RNA polymerase. In preferred embodiments, the RNA polymerase is a T7 polymerase, a SP6 polymerase, or a T3 polymerase. In other embodiments, the RNA polymerase promoter sequence is added at the ends of the bait sequences by reamplifying the bait sequences, such as by PCR or other nucleic acid amplification methods.

The sets of bait sequences produced according to the foregoing methods are useful in the methods of selection of subgroups of nucleic acids described herein.

Also provided are methods for determining the presence of a nucleic acid sequence, cell, tissue or organism in a sample. These methods include obtaining a sample containing nucleic acids and subjecting the nucleic acids in the sample to solution-based selection of nucleic acids according to the methods described herein. Alternatively, the nucleic acids can be sequenced after selection as described elsewhere herein. The presence or sequence of one or more nucleic acids of the subgroup of nucleic acids obtained by selection is determined. The presence or sequence of the one or more nucleic acids indicates the presence (and optionally the sequence) of a nucleic acid sequence, cell, tissue or organism in the sample. Such methods are applicable in diagnostic methods, such as for determining the presence of a pathogen, disease, or contaminant in the sample.

The nucleic acid sequence, cell, tissue or organism can be a variety of nucleic acid sequences, cells, tissues or organisms, including bacterial cells, tumor cells or tissues, viruses, nucleic acids having one or more mutations or variations (e.g., single nucleotide polymorphisms (SNPs), germ line mutations, somatic mutations). For example, somatic mutation detection can include deep resequencing of genes in tumor/normals. In this example, deep single-molecule resequencing is used to detect the mutations in the background of normal DNA. The sample can be obtained from the environment, from a patient, from an archival sample, etc.

As described herein, the invention includes a variety of methods and products for capture of sequences using solution hybridization, e.g., using capture probes derived from synthetic long oligonucleotides. Exemplary applications of the methods and products of the invention including the following:
Resequencing of any arbitrarily defined portion of a previously sequenced reference genome for research or diagnostic purposes;
Exome-resequencing (wherein the exome is all exons in a genome, or exons from a panel of relevant genes, e.g., genes implicated in cancer);
Promoterome resequencing (wherein the promoterome is all promoters in a genome, or promoters from a panel of relevant genes, e.g., genes implicated in cancer);
Enhancerome resequencing (wherein the enhancerome is all enhancers in a genome, or enhancers from a panel of relevant genes, e.g., genes implicated in cancer);
5′ or 3′ UTRome resequencing;
TEZome (transposon exclusion zones) resequencing (including the epigenetically bivalent domains);
Resequencing of other sets of distinct genomic features (“omes”) that constitute less than 10% of the human genome (or other complex genomes);
Resequencing large contiguous genomic regions.

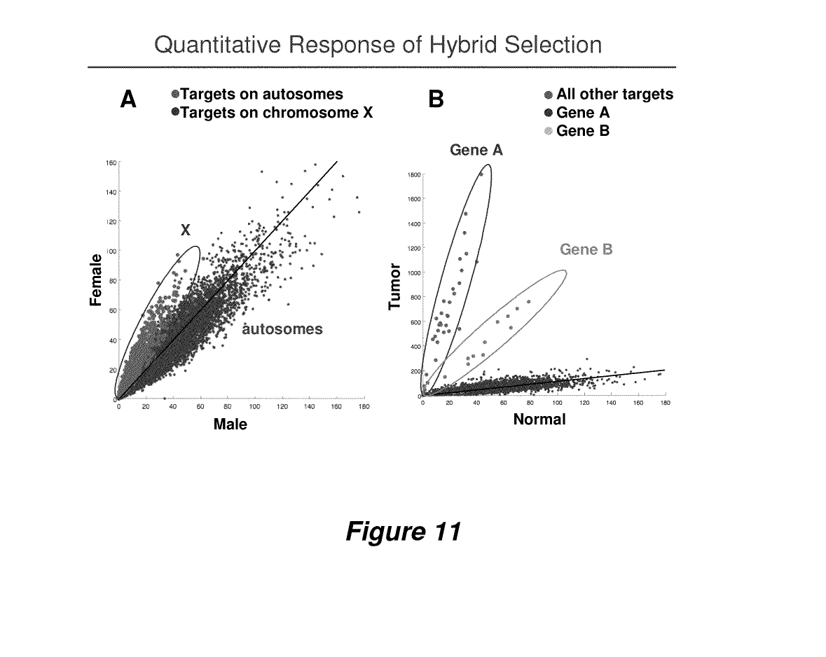

The methods described herein that include selection can be used for research purposes (e.g., resequencing a genomic regions implicated by genetic analysis to harbor disease-causing/modifying sequence variants) or for diagnostics purposes (e.g., resequencing all or a panel of relevant genes in a patient's DNA to aid in diagnosing the patient's condition). Additional uses include the following:
Capturing cDNAs for sequence analysis. cDNAs (first or 2^{nd} strand cDNA) are directly selected using the methods described herein. Capturing cDNAs using such methods will boost cDNAs derived from rare transcripts to levels that can be detected and re-sequenced with fewer reads than without selection. Hybrid selection will also reduce the representation of extremely abundant cDNAs, thus helping to normalize the representation of transcripts in the cDNA library. It is possible to use oligonucleotide-derived capture probes to remove unwanted cDNAs, either before or after the use of the bait sequences. This cDNA capture and sequencing method can be used for deep resequencing of a subset of the transcriptome for various purposes including mutation detection, detection of expressed fusion mRNAs, splice variants, mis-edited RNAs etc. This same approach can be used for analysis of RNA molecules. In this embodiment, DNA (or RNA) bait oligonucleotides are used to select RNA molecules, which then can be analyzed by reverse transcription and DNA sequencing.
Capturing sequences in archeological, forensic and other poorly conserved or heavily contaminated specimens that are refractory to “long-range” or even standard-range (>few hundred bp) PCR, e.g. Neanderthal bone, formaldehyde-fixed paraffin blocks. For example, one can use oligonucleotides for human sequences to enrich Neanderthal DNA from a library of DNA prepared from Neanderthal bones that contains mostly bacterial and other non-hominid DNA for more cost-effective sequencing of the Neanderthal genome (or portions thereof). This approach also can be used for analysis of other ancient DNA samples, and for analysis of modern, heavily contaminated samples, including but not limited to forensic materials obtained at a crime scene that may be contaminated with non-human DNA and therefore refractory to certain DNA diagnostic protocols.
Sequence analysis of tumor samples that are often preserved and archived in form of formaldehyde-fixed paraffin blocks. This form of preservation degrades nucleic acids, rendering the genomic DNA unsuitable for long-range PCR or other methods that require DNA that is more intact. Answering scientific questions that require sequencing long contiguous segment of the tumor genome can be extremely tedious and costly, if the only method for isolating templates for sequencing is standard short-range singleplex PCR with specific primers. It is known to those skilled in the art that it is possible to prepare small-insert fragment libraries (e.g., fragments carrying generic adapters that can be PCR amplified with generic primers) from such degraded DNA samples. Such small-insert libraries have been used for next-generation sequencing of tumor genomes. Hybrid selection, as described here, can be used to select a subgroup of nucleic acids from a small-fragment library that collectively cover a large genomic region in a form that is amenable to deep high-throughput sequencing.
DNA methylation analysis. For example, one can capture specific regions, and bisulfite resequence the captured material (e.g., using Illumina sequencing). Target “omes” include the CpG islands, the promoterome, the TEZome (especially the developmentally uncommitted, epigenetically bivalent domains).
Capturing viral sequences for sequence analysis (e.g., HIV sequences in random-primed cDNA from patient samples).
Capturing sequences in environmental samples for phylogenetically conserved sequences (e.g., 16S ribosomal RNA) and analyzing DNA by sequencing.
Capturing and identifying the sequences flanking transposon insertions, e.g., from a library of transposon-tagged bacteria. In similar embodiments, the methods described herein are used to capture and identify viral integration sites in the human genome (or other genome). For example, one could identify and sequence integration sites for hepatitis B virus by preparing baits specific for hepatitis B virus, selecting DNA fragments that contain hepatitis B viral DNA and sequencing the DNA fragments to determine the location in the genome and the sequence at which the virus integrated. This embodiment can be used for determining the integration sites of different viruses or known viral variants at the same time.
Detection of copy number variations. The finding that solution hybridization methods described herein are not only very consistent from sample to sample but also quantitative is completely unexpected. As shown in FIG. 11A, targets on chromosome X in female human DNA samples are recovered at about twice the rate than in male DNA samples, demonstrating the quantitative response of hybrid selection to copy number differences in the source DNA. More interestingly, as shown in FIG. 11B, by counting target sequences in tumor and normal samples one can identify target loci that are amplified (or under-represented) in the tumor relative to the normal. Selection of nucleic acid complexes for analyses of non-nucleic-acid constituents of the complexes. The complexes can be natural complexes (e.g., RNA-protein complexes formed in the cell) or artificial complexes (e.g., proteins that are tagged with one or more nucleic acids, even drugs and other chemicals). For example, one can use bait sequences as described herein to select all or a subset of non-coding long RNAs that have been crosslinked to proteins. The proteins then can be identified by mass spectrometry according to known standard methods. The RNA constituent can also be sequenced (after reverse transcription into DNA), thereby not only providing an internal control for the specificity of the selection, but also yielding information on the primary structure (e.g., splice forms) of the non-coding RNAs. In another example, one synthesizes a complex library of synthetic peptides (or any other chemical library) by combinatorial chemistry that are known to those skilled in the art such that each member of the chemical library is tagged with a specific oligonucleotides. The library of oligonucleotide-tagged peptides is mixed with a cellular extract for a time sufficient to permit binding of lipids (and/or other cellular constituents) to the peptides. One can then select one or more subgroups of peptides (by virtue of their known oligonucleotide tags) with oligonucleotide baits by solution hybridization. The lipids (or other biological class of molecules) bound and co-selected with the subgroup of oligonucleotide-tagged peptides are identified by HPLC or other analytical techniques according to known standard methods.
Subtractions. As those skilled in the art will appreciate, certain embodiments of the current invention can also be used as a method of depletion of unwanted sequences. Not intending to be bound by theory, to drive the hybridization towards completion, such experiments may require high bait concentrations and/or longer reaction times for complex subtraction targets than those used for hybrid selection. Examples include but are not limited to removing highly abundant cDNAs from cDNA libraries to allow deeper sampling and sequencing of less abundant cDNA sequences.

### EXAMPLES

### Example 1

### Hybrid Selection Protocol

### 1. Materials

1.1 Reagents, Enzymes and Kits

MAXIscript ® T7 Kit (Ambion, Cat # AM1312)
NucAway ™ Spin Columns (Ambion, Cat # AM10070)
TURBO DNAfree ™ kit (Ambion, Cat # AM1907)
Formaldehyde Sample Buffer (Lonza, Cat #50571)
FlashGel ™ RNA Cassettes
SUPERase · In ™ (Ambion, Cat # AM2694)
Biotin-16-uridine-5′-triphosphate (Roche, Cat # 11388908910)
RNA Century ™ Marker (Ambion, Cat # AM7780)
Qubit ™ fluorometer (Invitrogen, Cat # Q32857)
Qubit ™ assay tubes (Invitrogen, Cat # Q32856)
Quant-iT RNA Assay Kit (Invitrogen, Cat # Q32852)
Quant-iT DNA Assay Kit, Broad Range (Invitrogen, Cat # Q33130)
SSPE Buffer 20X Concentrate (Sigma, Cat # S2015)
Denhardt's Solution 50X Concentrate (Sigma, Cat # D2532)
Sodium dodecyl sulfate solution 10% (Sigma, Cat # L4522)
5M NaCl Solution (Ambion, Cat # AM9760G)
Nuclease-free Water (not DEPC-treated) (Ambion, Cat # AM9930)
20X SSC Solution (Ambion, Cat # AM9763)
1M Tris Solution, pH 8.0 (Ambion, Cat # AM9856)
0.5M EDTA Solution, pH 8.0 (Ambion, Cat # AM9260G)
Dynabeads ® M-280 Streptavidin (Invitrogen, Cat # 112-05D)
Phusion ™ High-Fidelity PCR Master Mix with HF Buffer
(NEB, Cat # F-531S)
Herculase ® II Fusion DNA Polymerase (Stratagene, Cat # 600675)
MinElute PCR Purification Kit (Qiagen, Cat # 28004)
QIAquick PCR Purification Kit (Qiagen, Cat # 28104)
FlashGel ™ DNA Cassette (Lonza, Cat # 57023)
FlashGel ™ DNA Marker (Lonza, Cat # 50473)
FlashGel ™ RNA Cassette (Lonza, Cat # 57027)
Agilent DNA 1000 chip
Solexa Library Construction Kit (Solexa, Cat.# 1002290)
10X T4 DNA ligase buffer with 10 mM ATP (NEB)
NuSieve ® GTG ® Agarose (Lonza, Cat # 50080)
Agarose, Molecular Biology grade (VWR, Cat # IB70042)
QIAquick Gel Extraction Kit (Qiagen, Cat # 28704)

1.2 Adapter Oligonucleotides and PCR Primers

AG3792
(SEQ ID NO: 1)
5′-TGTAACATCACAGCATCACCGCCATCAGTCxT-3′
(SEQ ID NO: 2)
AG3793
5′-[PHOS]GACTGATGGCGCACTACGACACTACAATGT-3′
AG3794
(SEQ ID NO: 3)
5′-ACATTGTAGTGTCGTAGTGCGCCATCAGTCxT-3′
AG2475
(SEQ ID NO: 4)
5′-GGATTCTAATACGACTCACTATAGGGATCGCACCAGCGTGT-3′
AG2454
(SEQ ID NO: 5)
5′-CGTGGATGAGGAGCCGCAGTG-3′
AG2888
(SEQ ID NO: 6)
5′-CTGGGAATCGCACCAGCGTGT-3′
AG3802
(SEQ ID NO: 7)
5′-CGCTCAGCGGCCGCAGCATCACCGCCATCAGT-3′
AG3803
(SEQ ID NO: 8)
5′-CGCTCAGCGGCCGCGTCGTAGTGCGCCATCAGT-3′

The “x” in oligonucleotides indicates a phosphorothioate linkage (x) between the last two nucleotides at the 3′ end that is resistant to excision by 3′-5′ exonucleases.

To anneal adapter oligonucleotides AG3792 and AG3793, they are mixed at 15 μM each in 10 mM Tris-HCl, pH 8, 10 mM NaCl and 0.1 mM EDTA, incubated for 2 min at 92° C. in a heat block and slowly cooled down to room temperature by switching off the heat-block. After 90-120 min cool down, the annealed adapter oligonucleotides are put on ice and stored in aliquots at −80° C.

1.3 Bait Oligonucleotide Library

Lyophilized pool of 10K, 22K or 55K synthetic 200mer oligonucleotides from Agilent. The oligonucleotides contain 170 target-specific bases (N₁₇₀) and 15 base universal tails on either end:

(SEQ ID NO: 9)
5′-ATCGCACCAGCGTGTN₁₇₀CACTGCGGCTCCTCA-3′

Some pools contain two 200mer oligonucleotides for each bait: the “plus” strand oligonucleotide above and its reverse complement:

(SEQ ID NO: 10)
5′-TGAGGAGCCGCAGTGN₁₇₀ACACGCTGGTGCGAT-3′

Synthesizing “plus” and “minus” oligonucleotides is meant as a precaution against synthesis failures due to base composition effects or difficult-to-synthesize sequences. “Plus” and “minus” oligonucleotides give rise to the same double-stranded PCR product when amplified with primers AG2888 and AG2454.

### 2. Protocol

2.1 “Pond” Library Preparation

1. Shear 3 μg of human genomic DNA in 100 μl of TE buffer for 4 min at 4° C. on a Covaris E210 instrument set to duty cycle 5, intensity 5, cycles/burst 200.

2. Concentrate sheared DNA sample using a MinElute PCR Purification Kit and elute the DNA in 26 μl EB buffer.

3. Analyze 1 μl of the eluted DNA sample on a Agilent DNA 1000 chip. The electropherogram should show a broad size distribution from ˜50 to 700 bp peaking between 200 and 300 bp.

4. With the remaining 25 μl set up a 100 μl end-repair reaction using reagents from the Solexa Library Construction Kit. When processing multiple samples, prepare a cocktail of all components except the DNA beforehand.

| Component | |
|---|---|
| Sheared and MinEluted DNA | 25 μl |
| Nuclease-free Water | 50 μl |
| 10X T4 DNA ligase buffer (with | 10 μl |
| 10 mM ATP) | |
| dNTP mix | 4 μl |
| T4 DNA polymerase | 5 μl |
| DNA polymerase I, Klenow fragment | 1 μl |
| T4 Polynucleotide kinase | 5 μl |

5. Incubate for 20 min at RT, clean the reaction using a Qiagen MinElute PCR Purification Kit and elute the sample in 32 μl EB buffer.

6. Using reagents from the Solexa Library Construction Kit, set up a 50 μl A-tailing reaction. When processing multiple samples, prepare a cocktail of all components except the DNA beforehand.

| Component | |
|---|---|
| End repaired and MinEluted DNA | 32 μl |
| 10X Klenow buffer | 5 μl |
| dATP | 10 μl |
| Klenow exo⁻ | 3 μl |

7. Incubate for 30 min at 37°, clean the reaction using a Qiagen MinElute PCR Purification Kit and elute the sample in 10 μl EB buffer.

8. Using reagents from the Solexa Library Construction Kit, set up a 50 μl adapter ligation reaction. When processing multiple samples, prepare a cocktail of all components except the DNA beforehand.

| Component | |
|---|---|
| A-tailed and MinEluted DNA | 10 μl |
| Nuclease-free Water | 4 μl |
| 2X DNA ligase buffer | 25 μl |
| 15 uM generic adapter (preannealed oligos AG3792 | 6 μl |
| and AG3793) | |
| DNA ligase | 5 μl |

9. Incubate for 30 min at RT, clean the reaction using a Qiagen MinElute PCR Purification Kit and elute the sample in 30 μl EB buffer.

10. Run adapter ligated and MinEluted sample along with a 100-base ladder size marker on a preparative 4% agarose gel (3:1 w/w mix of NuSieve® GTG® Agarose and Agarose, Molecular Biology grade) in 1×TAE buffer overnight at 23 V at 4° C. (cold room). Stain marker lane with SYBR green. Visualize size markers on a DarkReader and excise 280 to 380 bp region from the unstained preparative lane. (NOTE: A-tailing and adapters add 62 bases to the genomic DNA fragments. In practice, a 280-380 bp excision will produce somewhat smaller ˜260-360 bp products with ˜200-300 bp genomic inserts).

11. Weigh excised gel sliced. For each gram of agarose add 3 ml of buffer QG from the QIAquick Gel Extraction Kit. Melt the agarose by gentle agitation for 15 min at RT on a rotating bar. Once the agarose is solubilized, add 1 ml isopropanol for each g of agarose. Follow the instructions of the kit manufacturer and elute the adapter-ligated DNA in 50 μl EB buffer.

Steps 12-14 are optional.

12. Set up a 50 μl PCR reaction mix with 1 ul adapter-ligated DNA as template, using primers AG3792 and AG3794 and Phusion™ High-Fidelity PCR Master Mix with HF Buffer. Split the reaction mix into four 10 μl reactions on four 384-well PCR plates and run test PCR using a different number of PCR cycles for each plate as follows: 30 s/98° C.; 9, 12, 15, or 18 Cycles [10 s/98° C., 30 s/68° C., 45 s/72° C.]; 7 m/72° C.; ∞/4° C. Analyze the PCR reactions on a 1.2% Flash gel. 12 cycles of PCR is generally sufficient for amplifying the adapter-ligated library.

13. Set up a 200 μl PCR reaction mix with 4 μl of adapter-ligated library as template using primers AG3792 and AG3794 and Phusion™ High-Fidelity PCR Master Mix with HF Buffer. Split into four 50 μl in a 96-well PCR plate and run PCR as follows: 30 s/98° C.; 12 (or optimal number of) Cycles [10 s/98° C., 30 s/68° C., 45 s/72° C.]; 7 m/72° C.; ∞/4° C.

14. Stop by adding 1 ul of 0.5M EDTA per 50 μl aliquot. Clean up the reaction by one phenol/chloroform extraction followed by purification using QIAquick PCR Purification Kit.

15. Determine DNA concentration using Quant-iT™ DNA Assay Kit. Check library quality on agarose gel using FlashGel™ DNA Cassette and FlashGel™ DNA Marker as DNA Ladder. Unamplified adapter-ligated material is generally sufficient for one Hybrid Selection. 200 μl of PCR usually produces enough pond library material for four Hybrid Selections. Scaling up the volume of the PCR reaction is preferable to running more PCR cycles. 50 μl of unamplified adapter-ligated library is enough to set up fifty 50 μl PCR reactions often producing>25 μg of pond library. Larger amounts of pond library can be produced by using 0.1 μl instead of 1 μl of unamplified pond library as template and 15 instead of 12 PCR cycles.

2.2. Bait Preparation

1. Resuspend lyophilized oligonucleotide library from Agilent in 100 μl of low TE buffer (10 mM Tris-HCl, pH 8, 0.1 mM EDTA) and make 1:10 dilution (3 μl plus 27 μl low TE)

2. For each pool of oligonucleotides, set up two 50 μl PCR reaction mixes on ice, one with 1 μl diluted and one with 1 μl undiluted oligonucleotides using primers AG2454 and AG2888 (30 pmol each) and Herculase II Fusion. Split the reaction into 4×10 μl and run PCR in four 384 well plates using different number of PCR cycles for each plate: 2 m/95° C.; 9, 12, 15, or 18 Cycles [20 s/95° C., 30 s/50° C., 30 s/72° C.]; 7 m/72° C., ∞/4° C.

3. Run reactions on a 4% (NuSieve/regular 3:1) 0.5×TBE gel along with a 100 bp ladder size marker. Stain the gel with SYBR green to determine the optimum number of PCR cycles. The 21 base PCR primers increase the size of the universal tails from 15 to 21 bases on each end. The main PCR product should be 212 bp in size.

4. Set up scaled-up PCR reactions (4×50 ul) using the undiluted oligonucleotide library as library and run it in a 96-well PCR plate for the empirically determined number of PCR cycles.

5. Pool the four 50 ul aliquots, add 300 ul TE buffer and concentrate/desalt the PCR reaction on a Millipore Montage PCR-clean-up ultrafiltration cartridge (˜20 min at 1,000×g). Elute sample in 30 ul TE.

6. Run sample on a preparative 4% (NuSieve/regular 3:1) gel in 1×TAE for 3 hours at 50 V at RT. Stain entire gel with SYBR Green, visualize on a DarkReader and excise full-length (212 bp) PCR product band.

7. Weigh excised gel sliced. For each gram of agarose add 3 ml of buffer QG from the QIAquick Gel Extraction Kit. Melt the agarose by gentle agitation for 15 min at RT on a rotating bar. Once the agarose is solubilized, add 1 ml isopropanol for each g of agarose. Follow the instructions of the kit manufacturer and elute the amplified PCR product in 100 μl EB buffer.

8. Set up 50 μl PCR reaction mix with 1 μl of a 1:10 dilution of the gel-purified PCR product as template using primers AG2454 and AG2475 (30 pmol each) and Herculase II Fusion. Split the reaction into 4×10 μl and run PCR in four 384 well plates using different number of PCR cycles for each plate: 2 m/95° C.; 10, 13, 16, or 19 Cycles [20 s/95° C., 30 s/50° C., 30 s/72° C.]; 7 m/72° C.; ∞/4° C.

9. Run reactions on a 4% (NuSieve/regular 3:1) 0.5×TBE gel along with a 100 bp ladder size marker. Stain the gel with SYBR green to determine the optimum number of PCR cycles (usually 13 or 16 cycles). AG2475 contains the original 15 base universal tail, the T7 promoter plus additional 6 nucleotides at the 5′ end. The expected size of the PCR product is 232 bp.

10. Set up a preparative PCR (200 μl) PCR reaction with 4 μl of a 1:10 dilution of the gel-purified PCR product (step 9) as template using primers AG2454 and AG2475 (30 pmol each) and Herculase II Fusion. Split into four 50 μl aliquots and run PCR as follows: 2 m/95° C.; 13 (or optimal number of) Cycles [20 s/95° C., 30 s/55° C., 30 s/72° C.] 7 m/72° C.; ∞/4° C.

Stop by adding 1 ul of 0.5M EDTA per 50 μl aliquot. Clean up the reaction by MinElute Qiagen PCR purification kit. Determine DNA template concentration using Quant-iT™ DNA Assay Kit. Check DNA template on agarose gel using FlashGel™ DNA Cassette and FlashGel™ DNA Marker as DNA Ladder.

11. Set up the Transcription Reaction using MAXIscript® T7 Kit. Place the T7 RNA Polymerase on ice. Vortex the 10× Transcription Buffer and ribonucleotide solutions until they are completely in solution. Put the ribonucleotides on ice. Keep the 10× Transcription Buffer at room temperature (RT).

12. Assemble reaction at RT. Add Transcription Buffer after the water and template DNA.

| Component | |
|---|---|
| Nuclease-free Water to 20 μl | 6 μl |
| DNA template (500 ng) | 6 μl |
| 10X Transcription Buffer | 2.0 μl |
| 10 mM ATP | 1 μl |
| 10 mM CTP | 1 μl |
| 10 mM GTP | 1 μl |
| 10 mM UTP | 0.8 μl |
| 10 mM Biotin-16-UTP | 0.2 μl |
| T7 Enzyme Mix | 2.0 μl |

13. Pipette the mixture up and down and then centrifuge tube briefly. Incubate 1 hr 30 min at 37° C. Add 1 μl of 0.5 M EDTA to stop the reaction.

14. Remove unincorporated NTPs and salt load reaction on NucAway™ Spin Columns:

Re-hydrate the column with 650 μl of TE buffer at RT for 15 min. Spin the column at 750×g for 2 min and discard the wash tube. Apply the reaction sample (20 μl) to the center of the gel bed of the column. Place the column in the sample collection tube and spin at 750×g for 2 min. Remove sample from the collection tube.

15. Remove DNA template by using TURBO DNAfree™ kit:

Add 0.1 volume of 10× TURBO DNase Buffer and 1 μl TURBO DNase to the RNA sample and mix. Incubate at 37° C. for 15 min.

16. Add 0.1 volume of resuspended DNase Inactivation Reagent (at least 2 μl) and mix well and

incubate 2 min at RT, mixing the contents 2-3 times.

17. Pellet the DNase Inactivation Reagent at 10,000×g for 1.5 min and remove the supernatant, which contains the RNA Bait.

18. Determine RNA concentration using Quant-iT RNA Assay Kit.

19. Check RNA quality on gel using FlashGel™ RNA Cassette. Combine 2.5 μl diluted Formaldehyde Sample Buffer and 2.5 μl of RNA sample. Denature 2 minutes at 65° C. and load on the gel. Use RNA Century™ Marker as RNA Ladder.

20. Add 1 ul of SUPERase•In™ (20 U/ul) to RNA Bait for RNA protection and store biotinylated RNA at −70° C.

2.3 Hybrid Selection

1. Prepare RNA Baits and Blocking Agent/“Pond” Library for hybridization. Adjust RNA Baits concentrations to 500 ng in 5 ul. Add 1 ul of SUPERase•In™ to 5 ul of RNA Bait (total 6 ul). Adjust “Pond” Library concentration to 500 ng in 2.0 ul. For each hybridization reaction mix 2.0 ul of Targeted Library with 2.5 ul of Human Cot-1 DNA with concentration 1 ug/ul and 2.5 ul of Salmon Sperm DNA with concentration 1 ug/ul. Prepare 1 ml of 2× Hybridization Buffer: 0.5 ml 20×SSPE+0.2 ml 50×Denhardt's+20 ul 10% SDS+20 ul 0.5 M EDTA+0.24 ml H₂O.

2. Put Blocking Agent/“Pond” Library (7.0 ul) in a well of PCR plate. Put 50 ul of Hybridization Buffer in a separate well of same PCR plate. Incubate PCR plate 5 m/95° C.; 5 m/65° C. Put 6.0 ul of RNA Bait in a well of a separate PCR plate for 2 min at 65° C. Combine RNA Bait and 13 ul of Hybridization Buffer with 7.0 ul Blocking Agent/“Pond” Library at 65° C. Final reaction volume is 26 ul.

3. Incubate PCR plate at 65° C. for 66-70 hours

4. For capture of hybrid selected library wash In a 1.5-ml microcentrifuge tube 3.3×10⁷ (50 ul) of streptavidin-coated beads (Dynabeads® M-280 Streptavidin) three times in 200 ul Streptavidin bead binding buffer: 10 mM Tris-HCl (pH 7.5), 1 mM EDTA (pH 8) and 1 M NaCl. After each wash, remove the beads from the binding buffer with a Dynal magnetic separator. Resuspend the beads in 200 ul Streptavidin bead binding buffer and add the hybridization reaction (total reaction volume of 26 ul might be reduced after hybridization) to the bead suspension. Carry out binding at RT for 30 min with periodic mixing. Remove the beads from the binding buffer using a Dynal magnetic separator and discard the supernatant.

5. Wash the beads once, at RT for 15 min, in 0.5 ml of 1×SSC with 0.1% SDS.

Remove the beads from the binding buffer using a Dynal magnetic separator.

6. Wash the beads three times, each time at 65° C. for 10 min, in 0.5 ml 0.1×SSC with 0.1% SDS pre-warmed at 65° C.

7. To elute the hybridized DNA add 0.05 ml of 0.1 M NaOH to the beads and incubate at RT for 10 min. Remove the beads from the elution mixture using a Dynal magnetic separator. Transfer the supernatant to a 1.5-ml microcentrifuge tube containing 0.05 ml of 1 M Tris-HCl (pH 7.5) and desalt the solution by spin-column Min Elute Qiagen PCR purification kit to obtain ˜15 ul of Post Hybridization Template.

8. Set up Post-Hybridization PCR with primers AG3802 and AG3803 using Phusion™ High-Fidelity PCR Master Mix. Use 0.5 ul of Post Hybridization Template per 50 ul PCR reaction. 30 s 98° C.; 18 Cycles [10 s/98° C., 30 s/55° C., 30 s/72° C.]; 7 m/72° C., ∞/4° C. Clean up the reaction by purification using QIAquick PCR Purification Kit. Determine DNA concentration using Quant-iT™ DNA Assay Kit. Check library quality on agarose gel using FlashGel™ DNA Cassette and FlashGel™ DNA Marker as DNA Ladder.

2.4. Shotgun Sequencing

The Post-Hybridization PCR product is submitted for shotgun next-generation sequencing. Briefly, the PCR product is digested with NotI (to create “sticky” ligatable ends), cleaned up, and self-ligated at high concentration and run on a preparative gel. Concatenated ligation products>2 kb are extracted from the gel, sheared to 50-500 bp fragments, end-repaired, A-tailed, ligated to standard sequencing adapters, size selected, PCR-amplified and sequenced using the standard sequencing protocol.

### Example 2

### Solution Hybrid Selection with Ultra-Long Oligonucleotide Probes for Massively Parallel Targeted Sequencing

The development and commercialization of a new generation of increasingly powerful sequencing methodologies and instruments¹⁻⁴ has lowered the cost per nucleotide of sequencing data by several orders of magnitude. Within a short time, several individual human genomes have been sequenced on “next-generation” instruments^{3,5-7}, with plans and funding in place to sequence more (www.1000genomes.org).

Sequencing entire human genomes will be an important application of next-generation sequencing. However, many research and diagnostic goals may be achieved by sequencing a specific subset of the genome in large numbers of individual samples. For example, there may be substantial economy in targeting the protein-coding fraction, the “exome”, which represents only ˜1% of the human genome. The economy is even greater for many key resequencing targets, such as genomic regions implicated by whole-genome association scans and the exons of sets of protein-coding genes implicated in specific diseases. Efficient and cost-effective targeting of a specific fraction of the genome could substantially lower the sequencing costs of a project, independent of the sequencing technology used.

Sequencing targeted regions on massively parallel sequencing instruments requires developing methods for massively parallel enrichment of the templates to be sequenced. Recognizing the inadequacy of traditional single- or multi-plex PCR for this purpose, several groups have developed “genome-partitioning” methods for preparing complex mixtures of sequencing templates that are highly enriched for targets of interest⁸⁻¹⁵. Only two of these methods have been tested on target sets complex enough to match the scale of current next-generation sequencing instruments.

The first method, microarray capture^{9,12,13}, uses hybridization to arrays containing synthetic oligonucleotides matching the target sequence to capture templates from randomly sheared, adaptor-ligated genomic DNA; it has been applied to more than 200,000 coding exons¹². Array capture works best for genomic DNA fragments that are ˜500 bases long¹², thereby limiting the enrichment and sequencing efficiency for very short dispersed targets such as human protein-coding exons that have a median size of 120 bp¹⁶.

The second method, multiplex amplification¹⁴, uses oligonucleotides that are synthesized on a microarray, subsequently cleaved off and PCR-amplified, to perform a padlock and molecular-inversion reaction^{17,18} in solution where the probes are extended and circularized to copy rather than directly capture the targets. Uncoupling the synthesis and reaction formats in this manner is an advantage in that it allows re-using and quality testing of a single lot of oligonucleotide probes. However, the padlock reaction is far less understood than a simple hybridization and has not been properly optimized for this purpose. At the time of publication¹⁴, multiplex amplification missed more than 80% of the targeted exons in any single reaction and showed highly uneven representation of sequencing targets, poor reproducibility between technical replicates, and uneven recovery of alleles. A more recent non-sequencing-based study using a similar approach suggests that the uniformity, reproducibility and efficiency of multiplex amplification can be improved¹⁵.

Here we describe a new method, developed independently, that overcomes some of the weaknesses of previous methods. It combines the simplicity and robust performance of oligonucleotide hybridization with the advantages of amplifying array-synthesized oligonucleotides and performing the selection reaction in solution.

Sequencing targeted genomic loci using massively parallel technology requires new methods to capture the templates to be sequenced. We developed a capture method that uses biotinylated RNA “baits” to “fish” targets out of a “pond” of DNA fragments. The RNA baits are transcribed from PCR-amplified oligodeoxynucleotides originally synthesized on a microarray. This generates sufficient bait for multiple captures at concentrations high enough to drive the hybridization. We tested this method with 170-mer baits that target>15,000 coding exons and four genomic regions (1.7 Mb total) using Illumina sequencing as read-out. About 90% of bases that aligned uniquely to the genome fell within 500 bases of bait sequence; up to 50% lay on exons proper. The uniformity was such that ˜60% of target bases in the exonic “catch”, and ˜80% in the regional catch, had at least half the mean coverage. Our method which combines the economy of array-based oligonucleotide synthesis with the favorable kinetics of solution hybridization enables efficient and accurate resequencing of “exomes” and megabase-sized regions.

### Results

### Hybrid Selection Method

We developed a method for capturing sequencing targets that combines the flexibility and economy of oligonucleotide synthesis on a microarray with the favorable kinetics of hybridization in solution (see FIG. 1 and FIG. 3). A complex pool of ultra-long 200-mer oligonucleotides is synthesized in parallel on an Agilent microarray and then cleaved from the array. Each oligonucleotide consists of a target-specific 170-mer sequence flanked by 15 bases of a universal primer sequence on each side to allow PCR amplification. After the initial PCR, a T7 promoter is added in a second round of PCR. We then use in vitro transcription in the presence of biotin-UTP to generate a single-stranded RNA hybridization “bait” for “fishing” targets of interest out of a “pond” of randomly sheared, adaptor-ligated and PCR-amplified total human DNA. The hybridization is driven by the vast excess of RNA baits that cannot self-anneal. The “catch” is pulled-down with streptavidin-coated magnetic beads, PCR-amplified with universal primers, and analyzed on a “next-generation” sequencing instrument. The method allows preparation of large amounts of bait from a single oligonucleotide array synthesis that can be quality control tested, stored in aliquots and used repeatedly over the course of a large-scale targeted sequencing project.

### Capturing and Sequencing Exon Targets

For a pilot study, we selected a set of 1,900 human genes randomly chosen to ensure unbiased sampling regardless of length, repeat content or base composition. We designed 22,000 “bait” sequences of 170 bases in length, targeting all of the 15,565 protein-coding exons of these genes. The baits were tiled without overlap or gaps such that the entire coding sequence was covered (see Methods). This simple design minimizes the number of synthetic oligonucleotides required; for 75% of all coding exons in the human genome, a single oligonucleotide would be sufficient. As the median size of protein-coding exons is only 120 bp¹⁶, many baits extend beyond their target exon. Our test baits for catching exons constituted 3.7 Mb, and the targeted exons comprised 2.5 Mb (67%).

Our “pond” consisted of genomic DNA, derived from a human cell line (Coriell NA15510), that had been randomly sheared, ligated to standard Illumina sequencing adapters, size-selected to 200-350 bp (mean insert size˜250 bp), and PCR-amplified for 12 cycles. We hybridized 500 ng of this whole-genome fragment library with 500 ng biotinylated RNA bait, PCR-amplified the hybrid-selected DNA and generated 36-base sequencing reads off the Illumina adaptor sequence at the ends of each fragment. We obtained 85 Mb of sequence that aligned uniquely to the human genome; 76 Mb was on or within 500 bp of a bait.

Of the specifically captured 76 Mb of sequence, 49 Mb (65%) lay directly on a bait. The proportion of this sequence directly within the exons (36 Mb total) closely matched the proportion of exonic sequence within the bait. Overall, 58% and 42% of the 85 Mb uniquely aligning human sequence mapped to baits and exons, respectively.

The high stringency of hybridization selects for fragments that contain a substantial portion of the bait sequence. As a result, fragments for which both ends map near to or outside of the ends of the bait sequence are overrepresented relative to fragments that overlap less (that is, fragments that end near the middle of a bait). Merely end-sequencing the fragments with short 36-base reads therefore leads to elevated coverage near the end of the baits, with many reads falling outside the target, and a pronounced dip in coverage in the center. This effect is evident in the cumulative coverage profile representing 7,052 free-standing single-bait targets (FIG. 7A).

To improve coverage in the middle, we replaced end sequencing of the catch with shotgun sequencing of the catch. Specifically, we changed the Illumina adaptor on the whole-genome fragment library to a generic sequencing-platform independent adaptor and amplified the catch with PCR primers carrying a NotI site at their 5′ ends. NotI-digestion of the PCR product generates sticky ends and facilitates concatenation by co-ligation for subsequent re-shearing and shotgun sequencing of the hybrid selected DNA. This modification to the protocol shifted the coverage to the middle (FIG. 6B). About 90 of 102 Mb unique human sequence (88%) aligned within 500 bases of a bait. The proportion of bait sequence in the specific catch (90 Mb) rose from 65% to 77% (69 Mb; 51 Mb thereof on exon). The fraction of bait and exon sequence in the uniquely aligning human Illumina sequence was 67% and 50%, respectively.

Although shearing the catch improved the proportion of bait sequence, the process adds an additional round of library construction with associated costs, amplification steps, and potential biases. It also generates reads containing uninformative adaptor sequence as a by-product. During the course of these experiments, it became possible to increase the sequence read length on the Illumina platform. We reasoned that simply increasing the read length would also increase coverage in the middle and thus obviate the need for shotgun-library construction. Indeed, we performed end sequencing of the very same catch that had produced the bimodal coverage profile shown in FIG. 7A, this time running 76-base instead of 36-base reads on an Illumina GA-II instrument. The longer reads resulted in a unimodal, center-weighted cumulative coverage profile (FIG. 7B). This lane generated 492 Mb of sequence that aligned uniquely to the genome, of which 445 Mb were on or near a bait. Of the specifically captured sequence, 321 Mb (72%) was directly on the bait itself and 235 Mb (53%) was contained within the exons. About 65% of the unique human sequence was on bait; 48% was on exons proper. The average coverage of bases was 86-fold within baits and 94-fold within coding exons.

The percentage of the uniquely aligning human sequence that falls on or near a bait (e.g., 445/492=90% for the 76-base end reads) provides an upper bound for estimating the specificity of hybrid selection. In this experiment, 358 Mb (42%) of the 851 Mb of raw sequence did not align uniquely to the human genome (Table 1) and were not considered. By comparison, typically ˜55% of raw bases in whole-genome-sequencing lanes do not align uniquely. The raw bases likely contain hybrid-selected human sequence that is not unique. The lower bound, assuming that all discarded sequence represented repetitive human background sequence rather than low-quality reads, was 445/851=52%. To obtain a more precise number, we aligned the raw reads again to the human genome, this time allowing multiple placements, and determined the fraction of all human alignable sequence that lay on or within 500 bp of a bait. Based on this calculation, our best estimate for the specificity of this catch was 82%.

Of note, the specifically captured sequence included near-target hits that were not on exons proper. The percentage of uniquely aligning Illumina sequence that actually lay on coding sequence, i.e., the upper bound of the overall specificity of targeted exon sequencing, was 48% in this experiment. Table 1 shows a detailed breakdown of raw and uniquely aligned Illumina sequences and measures of specificity for the three targeted exon-sequencing experiments.

TABLE 1
Detailed breakdown of Illumina sequences generated from exon catches

| | 36-base GA-I | 36-base GA-I | 76-base GA-II |
|---|---|---|---|
| | end | shotgun | end |
| Length and kind of Illumina sequencing reads | sequences | sequences | sequences |
| Aggregate length of target^{a} | 2.5 Mb | 2.5 Mb | 2.5 Mb |
| Aggregate length of baits | 3.7 Mb | 3.7 Mb | 3.7 Mb |
| Total raw unfiltered sequence | 152 Mb | 219 Mb^{b} | 851 Mb |
| Raw sequence not aligned uniquely to genome^{c} | 67 Mb | 116 Mb | 358 Mb |
| Uniquely aligned human sequence | 85 Mb | 102 Mb | 492 Mb |
| Uniquely aligned sequence on target | 36 Mb | 51 Mb | 235 Mb |
| Uniquely aligned sequence near target^{d} | 40 Mb | 38 Mb | 210 Mb |
| Uniquely aligned sequence on or near target | 76 Mb | 90 Mb | 445 Mb |
| Fraction of uniquely aligned sequence on or near | 89% | 88% | 90% |
| target^{e} | | | |
| Fraction of raw bases uniquely aligned on or near | 50% | 41%^{g} | 52% |
| target^{f} | | | |
| Fraction of uniquely aligned bases on target^{h} | 42% | 50% | 48% |

^{a}Protein-coding exon sequence only.
^{b}Each unit of concatenated catch contains 44-46 bases (~18%) of generic adaptor sequence. Therefore, ~18% (39 Mb) of the 219 Mb is not of human origin.
^{c}All raw sequence that fails to align uniquely to the human reference genome including low-quality sequence.
^{d}Outside but within 500 bp of a target exon.
^{e}Upper bound for estimating the specificity of hybrid selection.
^{f}Lower bound for estimating the specificity of hybrid selection.
^{g}The denominator (219 Mb) includes ~39 Mb of sequence from the generic adapters. Excluding these 39 Mb, the lower bound for the estimated specificity of this catch is 90/180 = 50%.
^{h}Upper bound for the overall specificity of targeted exon sequencing.

### Regional Capture and Sequencing

Next, we designed and tested a pool of 170-mer baits for targeted sequencing of four genomic regions ranging from 0.22 to 0.75 Mb in size (Table 2). The combined span of the regions was 1.68 Mb. The target regions included a large portion of ENCODE region ENr113 as well as the genes IGF2BP2, CDKN2A and B, and CDKAL1. For a pilot experiment, we designed non-overlapping 170-mers that largely excluded repetitive sequence (allowing no more than 40 bases of repetitive sequence in each). The baits totaled 0.75 Mb in length, while the remaining 0.93 Mb was not covered owing to repetitive sequence content. We fished in a pond containing 350-500 bp fragments of DNA from the human cell line GM15510. The catch was analyzed with the shotgun sequencing approach above, with 36-base reads. The experiment preceded the development of the 76-base reads.

TABLE 2
Genome segments targeted for regional capture

| Locus | NCBI Build 35 (hg17) coordinates | Length (bp) |
|---|---|---|
| ENr113 | chr4: 118,629,614-119,072,769 | 443,156 |
| IGF2BP2 | chr3: 186,812,974-187,078,486 | 265,513 |
| CDKN2A + B | chr9: 21,937,304-22,155,946 | 218,643 |
| CDKAL1 | chr6: 20,617,611-21,368,293 | 750,682 |
| total bp | | 1,677,994 |

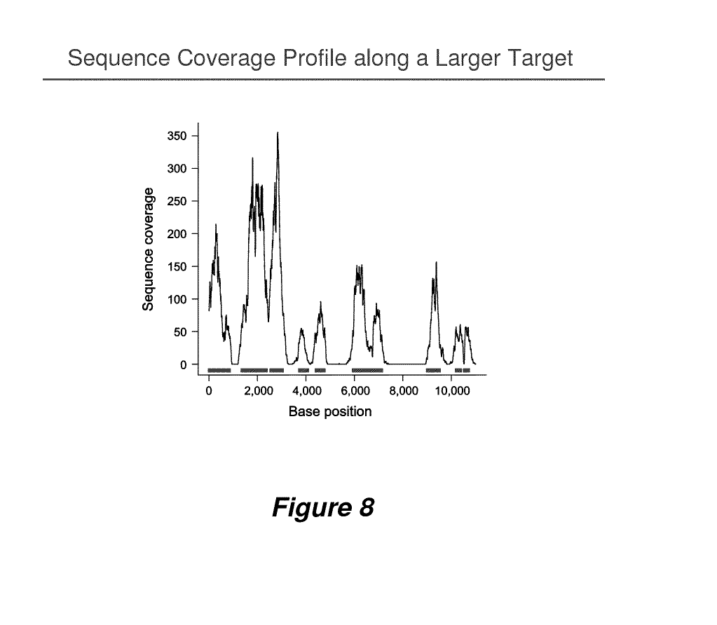

We generated one lane of IIlumina GA-I sequence, yielding 191 Mb that aligned uniquely to the human reference sequence. Of this sequence, 179 Mb (94%) fell within the four targeted genome segments. About 164 Mb were on bait whereas 15 Mb aligned uniquely within the 0.95 Mb that were not covered by baits. Essentially all unique sequence within the bait-free zones was within 500 bp of a bait sequence, suggesting that it had been caught by specific hybridization to a bait. A typical coverage profile along 11 kb is shown in FIG. 8. As expected, the coverage was not uniform and had peaks at unique segments that were represented in the bait pool and deep valleys or holes at mostly repetitive regions outside the baits. The average depth of coverage for the 0.75 million genome bases covered by bait in the four target regions was 221.

### Evenness of Coverage

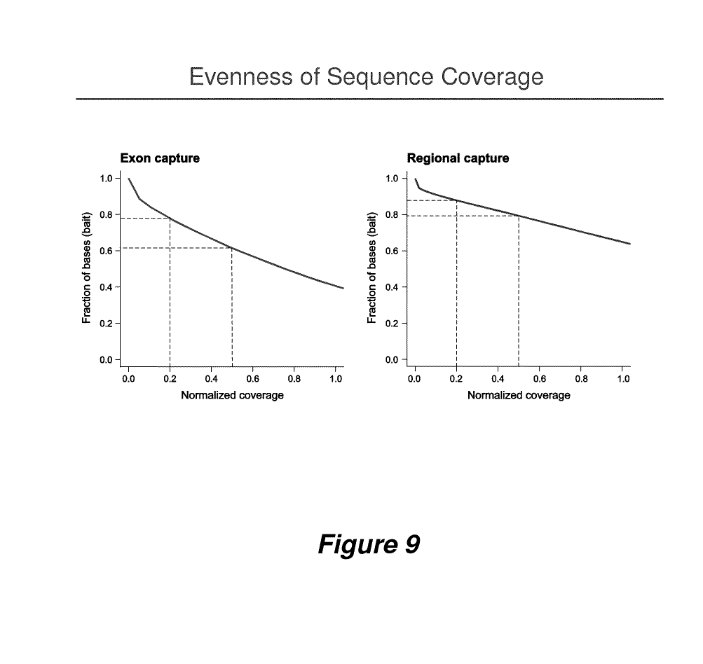

Uniformity of capture, along with specificity, is the main determinant for the efficiency and practical utility of any bulk enrichment method for targeted sequencing. The larger the differences in relative abundance, the deeper one has to sequence to cover the underrepresented targets. We sought to display the data in a form that is independent of the absolute quantity of sequence (FIG. 9). Specifically, we normalized the coverage of each base to the mean coverage observed across the entire set of targets. This allows comparison of results from experiments with widely differing sequence yields, different template preparation methods or different sequencing instruments.

The two graphs in FIG. 9 show the fraction of bases contained within a bait at or above a given normalized coverage level; the normalized coverage was obtained by dividing the observed coverage by the mean coverage, which was 18 for the shotgun-sequenced exon capture (FIG. 9, left panel) and 221 for the regional capture (FIG. 9, right panel). In the exon-capture experiment, more than 60% of the bases within baits achieved at least half the mean coverage, and almost 80% received at least one fifth. Twelve percent had no coverage in this particular sequencing lane. The normalized coverage-distribution plot for targeted regional sequencing is considerably flatter, indicating even better capture uniformity: 80% of the bases within baits received at least half the mean coverage; 86% received at least one fifth; 5% were not covered in this experiment.

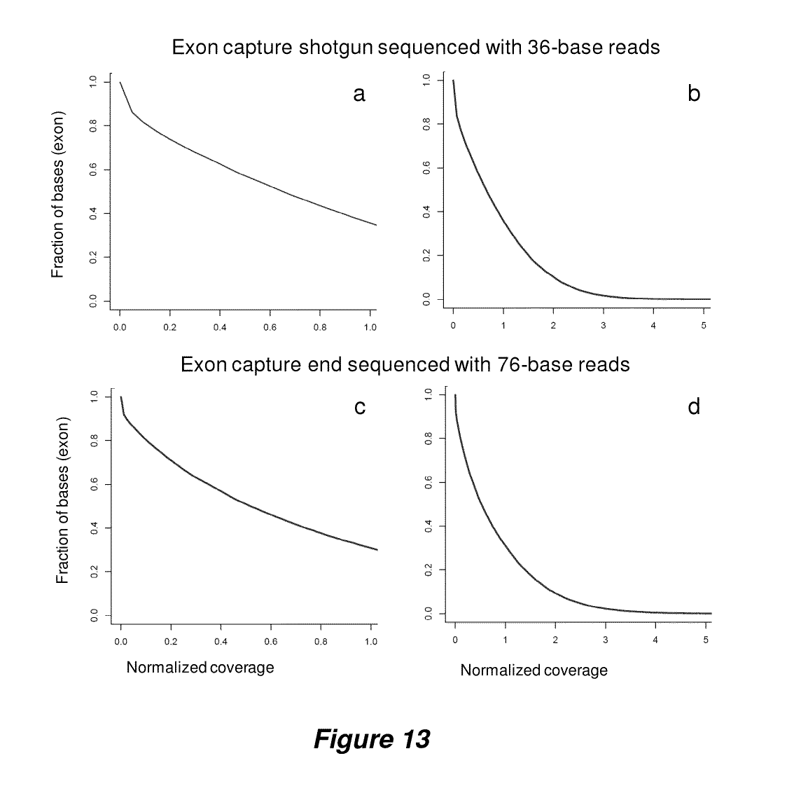

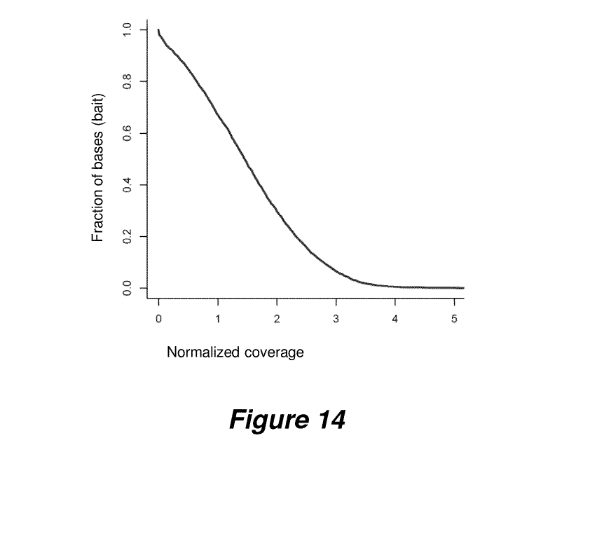

We attribute the differences in performance mainly to the fact that exon targets are generally short and isolated and often targeted by a single capture oligonucleotide (with few additional ones to choose from without widening the segment covered by bait). In contrast, the regional capture benefits from synergistic effects between adjacent baits, i.e., an overhanging genome fragment caught by one bait contributing to the sequence coverage underneath neighboring ones. The slightly longer DNA fragments used in this experiment (350-500 bases compared to 200-350 bases for exon capture) may have contributed to this effect. Additional coverage-distribution data, including graphs that were truncated at a normalized coverage of 5 instead of 1 to show the tail of the distribution, are available in FIGS. 13 and 14.

### Effects of Base Composition

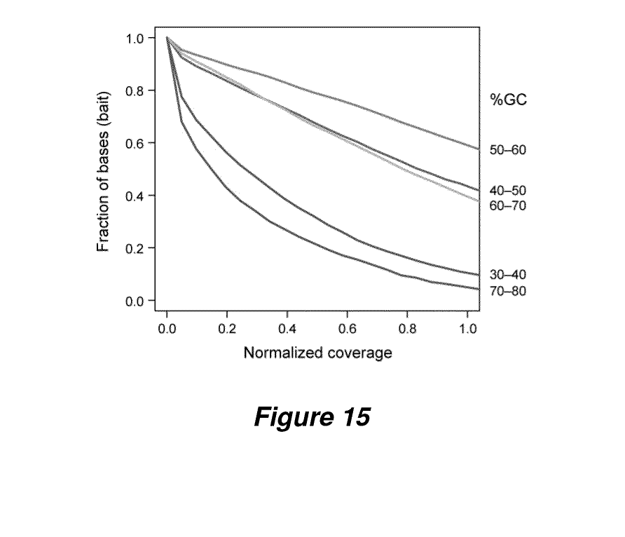

Separating the exon-capture baits into five categories based on their GC content revealed a systematic difference in coverage—with targets having GC content in the range of 50-60% receiving the highest coverage and those with very high (70-80%) or very low (30-40%) GC content getting the least coverage (see FIG. 15). The effects of base composition most likely reflect genuine systematic differences in hybridization behavior. However, it is also conceivable that GC bias at other steps in the process contribute to this effect. For example, we know from microarray assays that PCR can deplete oligonucleotide sequences with extreme base compositions up to ˜5-fold (data not shown). In addition, bias at the oligonucleotide-synthesis step may play a role. PCR amplification of the catch and sequencing itself is also known to introduce bias^{19,20}.

### Reproducibility

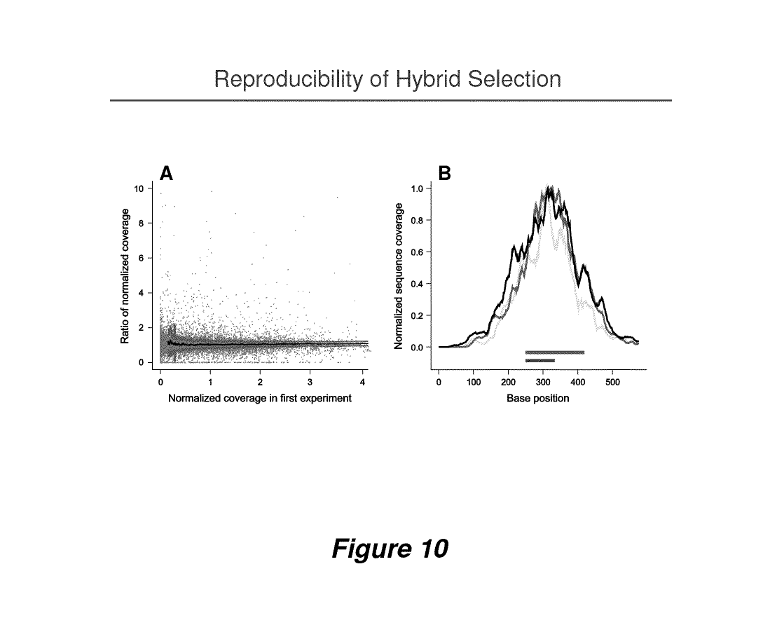

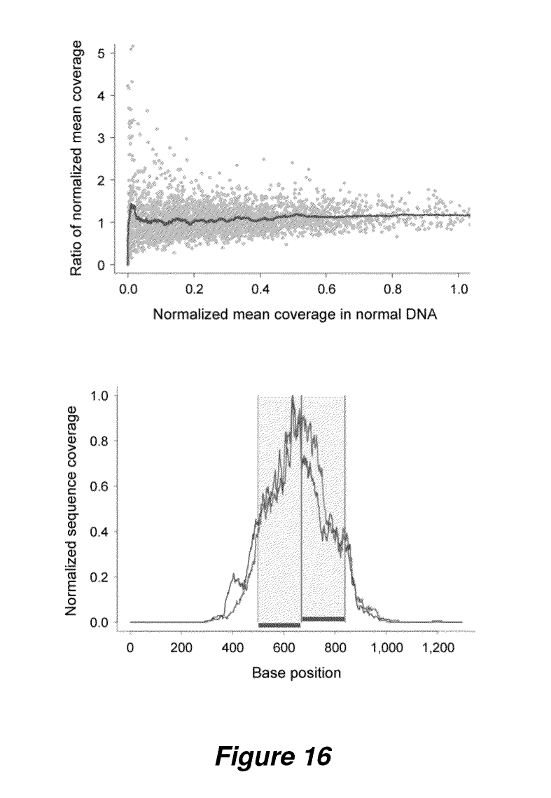

To assess the reproducibility of targeted exon sequencing we compared the results from independent technical replicates. Specifically, we performed two separate hybrid selections with ˜250 bp fragments prepared from the same source DNA (NA15510) and generated one lane of Illumina shotgun sequence each. As shown in FIG. 10A, the ratio of the mean normalized sequence coverage for individual exons in the two experiments was distributed closely around 1, indicating much less experiment-to-experiment than target-to-target variability. Base-by-base coverage profiles for individual exons were remarkably similar between the two technical replicates (purple and teal lines in FIG. 10B), consistent with the notion that variability in coverage is by and large systematic rather than stochastic. The coverage profile along the same exon in a different source DNA (Coriell NA 11994) followed a similar pattern (black line in FIG. 10B). Additional data that demonstrate the sample-to-sample consistency of targeted sequencing of whole-genome amplified DNA samples can be found in FIG. 16.

The number of exon positions where we called a high-confidence genotype (see Methods) in the two technical replicates was 1,586,379 and 1,578,975, respectively, i.e., ˜64% of the 2.5 Mb of targeted exon sequence. A total of 1,459,172 nucleotide positions were called in both. Of these, only 14 disagreed, indicating an overall discordance rate of ˜10⁻⁵ which is consistent with our threshold for genotype calls (LOD≧5).

The excellent reproducibility permits sequencing of essentially the same subset of the genome in different experiments. It also allows accurate predictions of target coverage at a given number of total sequencing reads. According to a normalized coverage distribution plot for exon as opposed to bait sequence (FIG. 13A), quadrupling the number of sequenced bases would increase the fraction of exon sequence called at high confidence to >80%. This can be easily achieved by longer reads and higher cluster densities on a newer Illumina GA-II instrument. Indeed, a single lane of 76-base end-sequencing reads provided high-confidence genotypes for 89% (2.2 Mb) of the targeted exon space.

### Accuracy of SNP Detection

To assess the accuracy of SNP detection, we fished for exons in three different human samples (Coriell NA11830, NA11992 and NA11994) that had been previously genotyped for the International HapMap project. With one lane of Illumina GA-I sequence for each sample, we were able to call 7,712 sequencing-based genotypes in coding exons for direct comparison with previously obtained genotypes. Each cell line had about 3,850 genotypes in HapMap within our target exons, of which ˜22% were heterozygous. As expected, the detection sensitivity of 67% (7,712 high-confidence genotype calls for 11,544 HapMap genotypes) closely matched the percentage of exon bases scanned with high confidence (64%) in these particular GA-I sequencing lanes.

The discordance rate at high-confidence sites was low (0.6%) and close to the estimated error rate of HapMap genotypes²¹. Of note, the HapMap discordancy for the very same loci in whole-genome Illumina sequencing experiments was essentially the same (0.6%). Hence, there is no evidence that the hybrid selection process per se compromises the accuracy.

To resolve a representative subset of the discrepancies we genotyped two DNA samples (NA11830 and NA11992) by mass-spectrometric primer-extension assays (Sequenom). A list of all 44 discordant genotypes plus 22 Sequenom genotypes is shown in Table 3. In 19 of 22 informative cases (86%), the Sequenom assay confirmed the sequencing-based result. Three cases were bona fide hybrid-selection sequencing errors that missed the non-reference allele at heterozygous positions. Bias against the non-reference allele may be due to preferential capture of the reference allele present in the capture probes, to preferential alignment against the reference genome or both.

TABLE 3
HapMap-discordant sequencing-based genotypes in target exons

| DNA | Position of SNP | Discordant | Reference | HapMap | Sequenom | Sequencing |
|---|---|---|---|---|---|---|
| sample | in hg17 | in >1 DNA | allele | genotype | genotype | genotype |
| NA11830 | chr1: 204603946 | 1 | C | G/G | C/C | C/C |
| NA11830 | chr3: 47291845 | | C | C/T | C/C | C/C |
| NA11830 | chr10: 88758232 | | A | A/G | A/G | A/A |
| NA11830 | chr11: 18151402 | 2 | C | C/G | C/C | C/C |
| NA11830 | chr11: 5329704 | | C | C/T | C/C | C/C |
| NA11830 | chr11: 60837716 | 3 | G | A/A | G/G | G/G |
| NA11830 | chr18: 2730713 | | A | A/A | A/G | A/G |
| NA11830 | chr22: 18343524 | | G | A/A | A/G | A/G |
| NA11830 | chrX: 152692839 | 4 | A | G/G | A/G | A/G |
| NA11830 | chr1: 111669558 | 5 | T | T/T | failed | C/T |
| NA11830 | chr7: 72188943 | 6 | G | A/A | failed | G/G |
| NA11830 | chr19: 40289301 | | A | A/G | failed | A/A |
| NA11830 | chrX: 152691986 | 7 | G | T/T | failed | G/G |
| NA11992 | chr1: 201111108 | | T | T/T | C/T | C/T |
| NA11992 | chr1: 204603946 | 1 | C | G/G | C/C | C/C |
| NA11992 | chr6: 134535089 | | G | A/G | G/G | G/G |
| NA11992 | chr6: 138775721 | | A | A/A | A/C | A/C |
| NA11992 | chr11: 18151402 | 2 | C | G/G | C/G | C/C |
| NA11992 | chr11: 36552169 | | G | A/G | G/G | G/G |
| NA11992 | chr11: 5798885 | | T | G/T | G/G | G/G |
| NA11992 | chr11: 60837716 | 3 | G | A/A | G/G | G/G |
| NA11992 | chr11: 71205343 | 8 | C | A/C | A/C | C/C |
| NA11992 | chr16: 360140 | | G | G/G | A/G | A/G |
| NA11992 | chr19: 59253603 | | G | G/G | A/G | A/G |
| NA11992 | chr21: 33536124 | | T | T/T | G/T | G/T |
| NA11992 | chrX: 152692839 | 4 | A | G/G | A/A | A/A |
| NA11992 | chr7: 72188943 | 6 | G | A/A | failed | G/G |
| NA11992 | chrX: 152691986 | 7 | G | T/T | failed | G/G |
| NA11994 | chr1: 111669558 | 5 | T | T/T | no data | C/T |
| NA11994 | chr1: 117371393 | | C | C/C | no data | C/T |
| NA11994 | chr1: 204603946 | 1 | C | G/G | no data | C/C |
| NA11994 | chr2: 79165915 | | T | C/T | no data | T/T |
| NA11994 | chr5: 146060861 | | C | A/A | no data | C/C |
| NA11994 | chr7: 72188943 | 6 | G | A/A | no data | G/G |
| NA11994 | chr11: 18151402 | 2 | C | C/G | no data | C/C |
| NA11994 | chr11: 5418566 | | C | A/C | no data | C/C |
| NA11994 | chr11: 5522626 | | C | A/C | no data | C/C |
| NA11994 | chr11: 60837716 | 3 | G | A/A | no data | G/G |
| NA11994 | chr11: 71205343 | 8 | C | A/C | no data | C/C |
| NA11994 | chr13: 75002303 | | G | G/T | no data | G/G |
| NA11994 | chr19: 1432850 | | G | A/A | no data | G/G |
| NA11994 | chr19: 59868073 | | A | A/A | no data | A/G |
| NA11994 | chr21: 32925800 | | A | A/A | no data | A/G |
| NA11994 | chrX: 152805038 | | A | G/G | no data | A/A |

All 44 discordant genotype calls between HapMap data and targeted exon-sequencing are shown. The total number of sequencing-based genotypes with HapMap data in Coriell DNA samples NA11830, NA11992 and NA11994 was 7,712 of which 6,668 were concordant. Discordancies were seen at 32 distinct SNP loci. Loci with discordant genotype calls in more than one DNA samples are numbered 1-8. To resolve a representative subset of the conflicting data, directed Sequenom SNP genotyping assays were developed and run on two of the DNA samples (NA11830 and NA11992). The resulting 22 Sequenom genotypes were considered the “truth” data. Bona fide errors in HapMap genotypes (N = 20) and in Illumina sequencing data (N = 3) are shown in red. All three sequencing-based miscalls are consistent with preferential capture of the reference allele.

Overall, the two alleles at heterozygous loci were represented almost equally on average. Based on 1,722 heterozygous SNP calls, the fraction of reads supporting the reference allele had a mean of 0.53 and a standard deviation of 0.12. The nearly balanced recovery of both alleles increases the power to detect heterozygotes. Consequently, the sensitivity to detect SNPs is mainly limited by sequence coverage rather than by systematic or stochastic allelic bias or drop-out effects.

### Discussion

We have developed a hybrid-selection method for enriching specific subsets of a genome that is flexible, scalable, and efficient. It combines the economy of oligodeoxynucleotide synthesis on an array with the favorable kinetics of RNA-driven hybridization in solution and works well for short dispersed segments and long contiguous regions alike. With further optimization, routine implementation of hybrid selection would enable deep targeted “next-generation” sequencing of thousands of exons as well as of megabase-sized candidate regions implicated by genetic screens. Hybrid-selection based targeting may be potentially useful for a variety of other applications as well, where traditional single-plex PCR is either too costly or too specific in that specific primers may fail to produce a PCR product that represents all genetic variation in the sample. Examples are enrichment of precious ancient DNA that is heavily contaminated with unwanted DNA, deep sequencing of viral populations in patient material, or metagenomic analyses of environmental or medical specimens.

Previous methods for hybrid selection have used cloned DNA, such as BACs or cosmids, to create capture probes for cDNA^{22,23} or genomic DNA fragments²⁴. Clone-based probes are suboptimal for several reasons. Readily available clones often contain extraneous sequences and are not easily configured into custom pools. Moreover, cDNAs are inefficient for capturing very short exons (data not shown). Instead of cloned DNA, we use pools of ultra-long custom-made oligonucleotides which are synthesized in parallel on a microarray and offer much greater flexibility. In principle, one can target any arbitrary sequence. As with all hybridization-based methods, repeat elements have to be either circumvented at the bait design stage or physically blocked during the hybridization. We currently do both. There are also fundamental limits to the power of hybridization to discriminate between close paralogs, members of gene families, pseudogenes, or segmental duplications.

We perform a simple pull-down with streptavidin-coated magnetic beads, a generic laboratory technique that does not require customized equipment. It can be performed in almost any tube or multi-well plate format, and there are numerous precedents for processing many samples in parallel. Our method is also largely independent of the sequencing platform. As shown here, it works well in combination with the Illumina platform whereby the hybrid-selected material can be either end-sequenced or shotgun sequenced. Direct end-sequencing with longer reads is clearly preferred as it is far less complex and requires fewer amplification steps. Our protocol can also be easily adapted for the 454 instrument (data not shown) which produces fewer but even longer reads, and, presumably, for other sequencing platforms as well.

The length of the baits allows thorough washes at high stringency to minimize contamination with non-targeted sequences that would cross-hybridize to the bait or hybridize to legitimate target fragments via the common adaptor sequence. A related source of background, indirect pull-down of repetitive “passenger” DNA fragments, is suppressed by addition of C0t-1 DNA to block repeats during the hybridization.

To prepare the bait, we amplify the complex pool of synthetic oligonucleotides twice by PCR. The risk of introducing bias during the amplification is more than compensated by its advantages: first, PCR selects for full-length synthesis products; second, it helps amortizing the fixed cost of chemical oligonucleotide synthesis over a large number of DNA samples; third and most importantly perhaps, it allows storage and testing at various stages of aliquots and obviates the need for frequent chemical re-synthesis and quality control of a given set of DNA oligonucleotides.

The sensitivity is in part due to the use of single-stranded RNA as capture agent. While a 5′-biotinylated double-stranded PCR product is equally specific (data not shown), it is not as good a hybridization driver. In a hybrid selection with single-stranded RNA, each bait is present in vast (several hundred-fold) excess over its cognate target. The excess RNA drives the hybridization reaction toward completion and reduces the amount of input fragment library needed. Further, saturating the available target molecules with an excess of bait prevents all-or-none single-molecule capture events that give rise to the stochastic and skewed representation of targets and alleles in multiplex amplification¹⁴. It also helps normalizing differences in abundance and hybridization rates of individual baits to some extent.

An important parameter for capturing short and dispersed targets such as exons is fragment size. Longer fragments extend beyond their baits and thus contain more sequence that is slightly off-target. On the other hand, shearing genomic DNA to a shorter size range generates fewer fragments that are long enough to hybridize to a given bait at high stringency. By virtue of the high excess of bait, our protocol works well for fishing in whole-genome libraries with a mean insert size of ˜250 bp, i.e., only slightly longer than the average protein-coding exon and minimum target size (164 and 170 bp, respectively). In contrast, microarray capture has a lower effective concentration of full-length probes, requires more input fragment library to drive the hybridization and becomes less efficient with input fragment libraries that have insert sizes much smaller than 500 bp¹². Array capture is therefore better suited for longer targets, for which edge effects and target dilution by over-reaching baits or overhanging fragment ends are negligible. In fact, capturing fragments larger than the oligonucleotides is beneficial for this application as it helps extend coverage into segments next to repeats that must be excluded from the baits. Because of synergistic effects between neighboring baits, contiguous regions are less demanding targets than short exons.

One advantage of hybrid selection is that long capture probes are more tolerant to polymorphisms than the shorter sequences typically used as primers for PCR or multiplex amplification. We have seen very little allelic bias and few cases of allelic drop out at SNP loci. The concordance of sequencing-base genotype calls and known HapMap genotypes was excellent (99.4%). For the majority of discrepancies that we looked at, the sequencing genotype was validated by a specific SNP-genotyping assay. We have not examined other genetic variation such as indels, translocations and inversions; the capture efficiency may be lower for such sequence variants because they differ more from the reference sequence used to design the baits.

In conclusion, the technology described here should allow extensive sequencing of targeted loci in genomes. Still, it remains imperfect with some unevenness in selection and some gaps in coverage. Fortunately, these imperfections appear to be largely systematic and reproducible. We anticipate that additional optimization, more sophisticated bait design based on physicochemical as well as empirical rules, and comprehensive libraries of pre-designed and pre-tested oligonucleotides will enable efficient, cost-effective, and routine deep resequencing of important targets and help identify biologically and medically relevant mutations.

### Methods

Capture Probes (“Bait”).

Libraries of synthetic 200-mer oligodeoxynucleotides were obtained from Agilent Technologies Inc. The pool for exon capture consisted of 22,000 oligonucleotides of the sequence 5′-ATCGCACCAGCGTGTN₁₇₀CACTGCGGCTCCTCA-3′ (SEQ ID NO:9) with N₁₇₀ indicating the target-specific bait sequences. Baits were tiled along exons without gaps or overlaps starting at the “left”-most coding base in the strand of the reference genome sequence shown in the UCSC genome browser (i.e., 5′ to 3′ or 3′ to 5′ along the coding sequence, depending on the orientation of the gene) and adding additional 170-mers until all coding bases were covered. The synthetic oligonucleotides for regional capture consisted of 10,000 200-mers that targeted 4,409 distinct 170-mer sequences, of which 3,227 were represented twice (i.e., the sequence above plus its reverse complement, SEQ ID NO:10) and 1,182 were represented thrice. For baits designed to capture a pre-defined set of targets, we first choose the minimal set of unique oligonucleotides and then add additional copies (alternating between reverse complements and the original plus strands) until the maximum capacity of the synthetic oligonucleotide array (currently up to 55,000) has been reached. The PCR product and the biotinylated RNA bait is the same for forward and reverse-complemented oligonucleotides. Synthesizing plus and minus oligonucleotides for a given target may provide better redundancy at the synthesis step than synthesizing the very same sequence twice, although we have no hard evidence that reverse complementing the oligonucleotides has any measurable benefit. Genome segments targeted for regional capture are shown in Table 2. Oligonucleotide libraries were resuspended in 100 μl TE0.1 buffer (10 mM Tris-HCl, 0.1 mM EDTA, pH 8.0). A 4-μl aliquot was PCR-amplified in 100 μl containing 40 nmol of each dNTP, 60 pmol each of 21-mer PCR primers A (5′-CTGGGAATCGCACCAGCGTGT-3′, SEQ ID NO:6) and B (5′-CGTGGATGAGGAGCCGCAGTG-3′, SEQ ID NO:5), and 5 units PfuTurboCx Hotstart DNA polymerase (Stratagene). The temperature profile was 5 min. at 94° C. followed by 10 to 18 cycles of 20 s at 94° C. ° C., 30 s at 55° C., 30 s at 72° C. The 212-bp PCR product was cleaned-up by ultrafiltration (Millipore Montage), preparative electrophoresis on a 4% NuSieve 3:1 agarose gel (Lonza) and QIAquick gel extraction (Qiagen). The gel-purified PCR product (100 μl) was stored at −70° C. To add a T7 promoter, a 1-μl aliquot was reamplified in 200 μl as before, except that the forward primer was T7-A (5′-GGATTCTAATACGACTCACTATAGGGATCGCACCAGCGTGT-3′, SEQ ID NO:4) and 12 to 15 PCR cycles were sufficient. Qiagen-purified 232-bp PCR product (1 μg) was used as template in a 100-μl MAXIscript T7 transcription (Ambion) containing 0.5 mM ATP, CTP and GTP, 0.4 mM UTP and 0.1 mM Biotin-16-UTP (Roche). After 90 min. at 37° C., the unincorporated nucleotides and the DNA template were removed by gel filtration and TURBO DNase (Ambion). The yield was typically 10-20 μg of biotinylated RNA as determined by a Quant-iT assay (Invitrogen), i.e., enough for 20-40 hybrid selections. Biotinylated RNA was stored in the presence of 1 U/μl SUPERase-In RNase inhibitor (Ambion) at −70° C.

Whole-Genome Fragment Libraries (“Pond”).

Whole-genome fragment libraries were prepared using a modification of Illumina's genomic DNA sample preparation kit. Briefly, 3 μg of human genomic DNA (Coriell) was sheared for 4 min. on a Covaris E210 instrument set to duty cycle 5, intensity 5 and 200 cycles per burst. The mode of the resulting fragment-size distribution was ˜250 bp. End repair, non-templated addition of a 3′-A, adaptor ligation and reaction clean-up followed the kit protocol except that we used a generic adaptor for libraries destined for shotgun-sequencing after hybrid selection. This adapter consisted of oligonucleotides C (5′-TGTAACATCACAGCATCACCGCCATCAGTCxT-3′ (SEQ ID NO:1) with “x” denoting a phosphorothioate bond resistant to excision by 3′-5′ exonucleases and D (5′-[PHOS]GACTGATGGCGCACTACGACACTACAATGT-3′, SEQ ID NO:2). The ligation products were cleaned up (Qiagen) and size-selected on a 4% NuSieve 3:1 agarose gel followed by QIAquick gel extraction. A standard prep starting with 3 μg of genomic DNA yielded˜500 ng of size selected material with genomic inserts ranging from ˜200 to ˜350 bp, i.e., enough for one hybrid selection. To increase the yield we typically amplified an aliquot by 12 cycles of PCR in Phusion High-Fidelity PCR master mix with HF buffer (NEB) using Illumina PCR primers 1.1 and 2.1, or, for libraries with generic adapters, oligonucleotides C and E (5′-ACATTGTAGTGTCGTAGTGCGCCATCAGTCxT-3′, SEQ ID NO:3) as primers. After QIAquick clean-up, if necessary, fragment libraries were concentrated in a vacuum microfuge to 250 ng per pi before hybrid selection.

Hybrid Selection.

A 7-μl mix containing 2.5 μg human C₀t-1 DNA (Invitrogen), 2.5 μg salmon sperm DNA (Stratagene) and 500 ng whole genome fragment library was heated for 5 min. at 95° C., held for 5 min. at 65° C. in a PCR machine and mixed with 13 μl prewarmed (65° C.) 2× hybridization buffer (10×SSPE, 10×Denhardt's, 10 mM EDTA and 0.2% SDS) and a 6-μl freshly prepared, prewarmed (2 min. at 65° C.) mix of 500 ng biotinylated RNA and 20 U SUPERase-In. After 66 h at 65° C., the hybridization mix was added to 500 ng (50 μl) M-280 streptavidin Dynabeads (Invitrogen), that had been washed 3 times and were resuspended in 200 μl 1M NaCl, 10 mM Tris-HCl, pH 7.5, and 1 mM EDTA. After 30 min. at RT, the beads were pulled down and washed once at RT for 15 min. with 0.5 ml 1×SSC/0.1% SDS, followed by three 10-min. washes at 65° C. with 0.5 ml prewarmed 0.1×SSC/0.1% SDS, resuspending the beads once at each washing step. Hybrid-selected DNA was eluted with 50 μl 0.1 M NaOH. After 10 min. at RT, the beads were pulled down, the supernatant transferred to a tube containing 70 μl 1 M Tris-HCl, pH 7.5, and the neutralized DNA desalted and concentrated on a QIAquick MinElute column and eluted in 20 μl. We routinely use 500 ng of “pond” and “bait” per reaction but have seen essentially identical results in proportionally scaled-down 5 μl reactions with 100 ng each.

“Catch” Processing and Sequencing.

For fragment libraries carrying standard Illumina adaptor sequences, 4 μl of hybrid-selected material was amplified for 14 to 18 cycles in 200 μl Phusion polymerase master mix and PCR primers 1.1 and 2.1 (Illumina) and the PCR product cluster-amplified and end-sequenced for 36 or 76 cycles. Hybrid-selected material with generic adaptor sequences (8 μl) was amplified in 400 μl Phusion High-Fidelity PCR master mix for 14 to 18 cycles using PCR primers F (5′-CGCTCAGCGGCCGCAGCATCACCGCCATCAGT-3′, SEQ ID NO:7) and G (5′-CGCTCAGCGGCCGCGTCGTAGTGCGCCATCAGT-3′, SEQ ID NO:8). Initial denaturation was 30 s at 98° C. Each cycle was 10 s at 98° C., 30 s at 55° C. and 30 s at 72° C. Qiagen-purified PCR product (˜1 μg) was digested with NotI (NEB), cleaned-up (Qiagen MinElute) and concatenated in a 20-μl ligation reaction with 400 U T4 DNA ligase (NEB). After 16 h at 16° C., reactions were cleaned up (Qiagen) and sonicated (Covaris). Sample preparation for Illumina sequencing followed the standard protocol except that the PCR amplification was limited to 10 cycles.

Genotyping.

Specific custom SNP genotyping was performed in 24-plex PCR and primer-extension reaction format using MassARRAY iPLEX chemistry and mass-spectrometric detection (Sequenom).

Computational Methods.

All coverage and SNP statistics are for single lanes of sequencing data. Illumina reads were collected from the instrument and aligned to the human genome using the Broad Institute's in-house aligner, which is the ImperfectLookupTable (ILT) of the ARACHNE2 genome assembly suite²⁵ and is available with documentation at www.broad.mit.edu/wga/ILT. Briefly, a lookup table of the locations of every 12-mer in the genome was computed. For a single read, each 12-mer in the read was looked up, and all occurrences of each 12-mer were considered putative placements. Each putative placement of the read in the genome was interrogated for number of mismatches. No insertions or deletions were considered. To ensure high quality and unique placements, only reads with 4 or fewer errors and a next-best placement at least 3 errors worse were considered. Coverage at each reference position was accumulated from the unique alignments. All aligned bases were included in the basic coverage calculations. High-confidence base calls (and coverage calculations based thereon) excluded bases that failed a signal clarity filter. The filter was that the ratio of brightest dye color to next-brightest dye color had to be 2 or greater. Typically, about 80% of aligned bases passed this filter. Genotypes at each position were inferred with a straightforward Bayesian model. The likelihood of the observed data P(data|genotype) assuming each genotype at each position were computed with the assumptions that each allele is equally likely to be observed and miscalls occur with a rate of 1/1000. These genotypes were combined with a prior probability over the genotypes defined by the reference. The prior used was: P(homozygous reference)=0.999, P(heterozygous ref/nonref)=0.001, P(nonref)=0.00001. This yields the posterior probability P(genotype|data). The most likely genotype was selected. The “confidence” in our call of the specific genotype was the ratio of the best to next-best theory. We used a best-to-next-best ratio of 10⁵ (LOD score 5) as threshold for calling a high-confidence genotype. The confidence in our belief that there was a SNP (independent of the specific genotype) was the ratio of the best theory to the reference. We used a best-to-reference ratio of 10⁵ as our minimum confidence cutoff for reporting a SNP. Genome coordinates are zero-offset and for NCBI Build 35 (hg17). Raw unaligned IIlumina sequences in SRF format from the hybrid-selection experiments described here are available at www.broad.mit.edu/wga/NBT.

### REFERENCES

1. Margulies, M. et al. Genome sequencing in microfabricated high-density picolitre reactors. Nature 437, 376-380 (2005).
2. Shendure, J. et al. Accurate multiplex polony sequencing of an evolved bacterial genome. Science 309, 1728-1732 (2005).
3. Bentley, D. R. et al. Accurate whole genome sequencing using reversible terminator chemistry. Nature 456, 53-59 (2008).
4. Smith, D. R. et al. Rapid whole-genome mutational profiling using next-generation sequencing technologies. Genome Res. (2008).
5. Ley, T. J. et al. DNA sequencing of a cytogenetically normal acute myeloid leukaemia genome. Nature 456, 66-72 (2008).
6. Wang, J. et al. The diploid genome sequence of an Asian individual. Nature 456, 60-66 (2008).
7. Wheeler, D. A. et al. The complete genome of an individual by massively parallel DNA sequencing. Nature 452, 872-876 (2008).
8. Dahl, F., Gullberg, M., Stenberg, J., Landegren, U. & Nilsson, M. Multiplex amplification enabled by selective circularization of large sets of genomic DNA fragments. Nucleic Acids Res. 33, e71 (2005).
9. Albert, T. J. et al. Direct selection of human genomic loci by microarray hybridization. Nat. Methods 4, 903-905 (2007).
10. Dahl, F. et al. Multigene amplification and massively parallel sequencing for cancer mutation discovery. Proc. Natl. Acad. Sci. USA 104, 9387-9392 (2007).
11. Fredriksson, S. et al. Multiplex amplification of all coding sequences within 10 cancer genes by Gene-Collector. Nucleic Acids Res. 35, e47 (2007).
12. Hodges, E. et al. Genome-wide in situ exon capture for selective resequencing. Nat. Genet. 39, 1522-1527 (2007).
13. Okou, D. T. et al. Microarray-based genomic selection for high-throughput resequencing. Nat. Methods 4, 907-909 (2007).
14. Porreca, G. J. et al. Multiplex amplification of large sets of human exons. Nat. Methods 4, 931-936 (2007).
15. Krishnakumar, S. et al. A comprehensive assay for targeted multiplex amplification of human DNA sequences. Proc. Natl. Acad. Sci. USA (2008).
16. Clamp, M. et al. Distinguishing protein-coding and noncoding genes in the human genome. Proc. Natl. Acad. Sci. USA 104, 19428-19433 (2007).
17. Nilsson, M. et al. Padlock probes: circularizing oligonucleotides for localized DNA detection. Science 265, 2085-2088 (1994).
18. Hardenbol, P. et al. Multiplexed genotyping with sequence-tagged molecular inversion probes. Nat. Biotechnol. 21, 673-678 (2003).
19. Dohm, J. C., Lottaz, C., Tatiana Borodina, T. & Himmelbauer, H. Substantial biases in ultra-short read data sets from high-throughput DNA sequencing. Nucleic Acids Res. 36, e105 (2008).
20. Quail, M. A. et al. A large genome center's improvements to the Illumina sequencing system. Nat Methods 5, 1005-1010 (2008).
21. Frazer, K. A. et al. A second generation human haplotype map of over 3.1 million SNPs. Nature 449, 851-861 (2007).
22. Lovett, M., Kere, J. & Hinton, L. M. Direct selection: a method for the isolation of cDNAs encoded by large genomic regions. Proc. Natl. Acad. Sci. USA 88, 9628-9632 (1991).
23. Parimoo, S., Patanjali, S. R., Shukla, H., Chaplin, D. D. & Weissman, S. M. cDNA selection: efficient PCR approach for the selection of cDNAs encoded in large chromosomal DNA fragments. Proc. Natl. Acad. Sci. USA 88, 9623-9627 (1991).
24. Bashiardes, S. et al. Direct genomic selection. Nat. Methods 2, 63-69 (2005).
25. Jaffe, D. B. et al. Whole-genome sequence assembly for mammalian genomes: Arachne 2. Genome Res 13, 91-96 (2003).

### Example 3

### Production Hybrid Selection Protocol

This example is the production protocol of the Broad Institute Genome Sequencing Platform. It is written for hybrid selection of 24 samples in parallel but can be easily scaled to 96 samples and hybrid selections. It uses lab automation stations (e.g., Velocity 11 Bravo Deck; Janus) at most of the individual steps. Briefly, the DNA sample is sheared, end-repaired, A-extended, size-selected (non-gel based double SPRI protocol), ligated to Illumina paired-end sequencing adapters, and PCR amplified. The PCR-amplified “pond” is hybridized to a biotinylated RNA bait. Biotinylated hybrids are captured and washed on the automated bead capture apparatus shown in FIG. 12. The catch is PCR amplified and paired-end-sequenced with 2×76-base Illumina reads according to standard methods.

### 1. Standard Automated Library Construction with Double-SPRI Size Selection

1.1 DNA Shearing on Covaris E210

1. Ensure that the Covaris has been degassed & the bath temp has reached ˜3 degrees.

2. Bring the volume of genomic DNA samples to 100 ul with 1×TE.

3. Load 24 100 ul glass shearing tubes in a staggered orientation into bottom half of the plastic holder.

4. Transfer all 100 ul of DNA to the tubes. Record the location of each GSSR sample in the holder.

5. Cap the shearing tubes with snap caps with blue septa.

6. Secure the top half of plastic holder on top of tubes.

7. Bring Covaris stage to loading position & load plate in proper orientation.

8. Open the “AFA_Vessel_{—}150” protocol. Settings: Duty cycle: 20%. Intensity: 5. Cycles per burst: 200. Time: 300 sec. Z Axis: 6 mm.

9. Click on “Configure” to check that wells containing tubes are selected for shearing.

10. Click “Start” to begin shearing.

11. Proceed to QiaQuick plate clean up.

1.2 Purification & Concentration (Qiaquick Plate)

1. Transfer 100 uL DNA to be purified in a 96-well deep well plate in a staggered fashion.

2. Add 5 volumes of Buffer PB to each sample. ie. For 100 ul reaction, add 500 ul PB. Vortex on and off 5 times to mix.

3. Prepare Qiaquick vacuum manifold: Place 48 well waste plate inside manifold. Place clear top ½ of manifold over waste plate. Ensure that hose is attached to house vacuum. Place Qia96 filter plate (yellow) in top of manifold.

4. Transfer 1200 ul of sample+PB to Qia96 plate w/ a Matrix 1250 ul multichannel pipette.

5. Turn on house vacuum. Press on manifold to ensure seal.

6. Allow all liquid to pass through filter. Repeat with additional sample if necessary.

7. To wash, add 1 ml of Buffer PE. Turn on vacuum & allow liquid to pass through filter.

8. Run vacuum for 2 min more to dry.

9. Seal the plate with sticky seal and allow the pressure to stabilize.

10. Fold 3 LARGE Kimwipes in half & lay on bench top in a row.

11. Remove seal with a ripping motion and turn vacuum off.

12. Remove Qia96 plate from manifold & tap up-side-down firmly ONCE per Kimwipe (total of 3 taps).

13. Turn vacuum on and repeat steps 9-12 three times.

14. Remove waste tray and dispose of it.

15. Place an Eppendorf 96 well PCR plate where waste plate was in the apparatus and put the filter plate on top in the same orientation. (ie. well A1 over well A1).

16. Dispense 40 ul of EB into Qiaquick plate directly onto the filter of each well

17. Allow the plate to sit for 2 min.

18. Turn vacuum on. Let plate sit under vacuum for 2 min.

19. Apply a plate seal, wait for pressure to build, then rip away smoothly.

20. Repeat step 19 a total of 3 times.

21. Stop vacuum and remove filter plate without making contact with PCR plate underneath.

22. Tap the PCR plate lightly on the table top to move DNA to bottom.

23. Seal the PCR plate with optical strip caps and spin down briefly.

24. Dispose of the filter plate and rinse the vacuum manifold for reuse.

25. Run standard pico green DNA quantitation assay. and Agilent Bioanalyzer assay to check size distribution.

1.3 End Repair

1. Starting material is 29 ul from Qia96 post shearing cleanup and Agilent QC.

2. Obtain the three tubes listed in table below.

| | | For 7 Rxn | | For 24 Rxn | |
|---|---|---|---|---|---|
| Reagent | Tube Name | Mix (ul) | | Mix (ul) | |
| 150 ul 10X T4 DNA | FRAGPOLMIX510 | 119 | ul | 510 | ul |
| Ligase Buffer | | | | | |
| 150 ul BSA (1 mg/ml) | | | | | |
| 150 ul ATP (10 mM) | | | | | |
| 60 ul dNTPs (10 mM) | | | | | |
| 150 ul T4 PNK | T4PNK150 | 35 | ul | 150 | ul |
| 150 ul T4 DNA Pol | T4POL3U150 | 35 | ul | 150 | ul |

| | | |
|---|---|---|
| Mix Total Volume | 189 | 810 |

3. Allow FRAGPOLMIX510 tube to thaw at room temp.

4. Transfer the T4PNK150 and T4POL3U150 tubes to a bench top cooler.

5. Prepare a master mix for 7 or 24 samples in 1.5 ml tube as shown in Table 1. Vortex 30 sec to mix. Spin down.

6. Add 20 ul of mix to each of the 24 samples in the 96-well PCR plate. Total volume will be 50 ul.

7. Cap the plate.

8. Transfer to thermal cycler and run the “End Repair” program: 12° C. for 15 min, 25° C. for 15 min, 4° C. hold.

9. Proceed to Single SPRI Cleanup.

1.4 SPRI Clean Up

1. Wipe down Bravo deck with EtOH.

2. Wash out all reservoirs with EtOH and water.

3. From the desktop open up “VWorks” next to the Velocity 11 Bravo.

4. Log in to software.

5. Open up the “Solexa” folder and the file called “SPRI_Cleanup.”

6. Make sure you mix the SPRI bead stock tube vigorously to resuspend the beads before use.

7. Pour some SPRI beads into a trough and pipette 150 ul of them into each well of an Eppendorf 96 well plate as seen in FIG. 3 and allow them to come to room temp before using. (return stock to fridge directly and dispose of the unused portion in trough).

8. Measure pH of SPRI. See “Appendix—Measuring pH of SPRI”.

9. Obtain the remaining materials and set up the Bravo deck.

10. Transfer 50 μL DNA to be purified in a 96-well Eppendorf plate in a staggered fashion.

11. Ensure that the water reservoir connected to the pump is full.

12. The program will run the wash station first. Abort the protocol if the water is not flowing. Restart the program until the wash is functioning.

FOLLOWING STEPS ARE AUTOMATED ON BRAVO

13. 120 μL of SPRI beads are added to each well.

14. Pipette mix 25× and incubate for 10 minutes.

15. Separate supernatant from beads by placing Eppendorf plate on Dynal magnet for 6 minutes; discard supernatant.

16. Leave plate on magnet and wash beads with 60 μL of 70% Ethanol.

17. Move Eppendorf plate off magnet & air dry SPRI beads for 6 minutes at room temp.

18. Add 40 μL of Tris-HCl to each well.

19. Pipette mix 5× and incubate for 3 minutes at room temp.

20. Separate liquid from beads on magnet for 6 minutes.

21. Transfer 40 μL eluate into destination 96 well Eppendorf plate.

1.5 A Base Addition

1. Starting material is 40 ul elutions from SPRI post end repair cleanup in 96 well plate.

2. Allow ABASEADDMIX tube to thaw at room temperature.

3. KLENOWEXOAQ tube should remain in a bench top cooler.

4. Prepare Master mix for 7 or 24 reactions in a 1.5 ml tube as shown in Table below. Vortex briefly to mix.

| | | | |
|---|---|---|---|
| | | For 7 Rxn | For 24 Rxn |
| Reagent | Tube Name | Mix (ul) | Mix (ul) |

| | | | | | |
|---|---|---|---|---|---|
| 60 ul Nuclease Free | ABASEADDMIX | 119 | ul | 510 | ul |
| Water | | | | | |
| 150 ul 10X Klenow | | | | | |
| Buffer | | | | | |
| 300 ul dATP (1 mM) | | | | | |
| 90 ul Klenow exo− | KLENOWEXOAQ | 21 | | 90 | ul |
| Mix Total Volume | | 140 | | 600 | |

5. Add 15 ul of master mix to each of the samples in the 96 well plate for a total volume of 55 ul for each sample, pipette mixing each sample as the master mix is added.

6. Cap the plate.

7. Transfer to thermocycler and run A Addition protocol (37° C. for 30 min) for a plate.

8. Proceed to double sided SPRI cleanup/size selection if performing automated library construction.

1.6 Size Selection Double Sided SPRI

1. Wipe down Bravo deck with EtOH.

2. Wash out all reservoirs with EtOH and water.

3. From the desktop open up “VWorks” next to the Velocity 11 Bravo.

4. Log in to software.

5. Open up the “Solexa” folder and the file called “Double SPRI_Cleanup.”

6. Make sure to mix the SPRI bead stock tube vigorously to resuspend the beads before use.

7. Pour some SPRI beads into a trough and pipette 200 ul of them into 24 staggered wells of an Eppendorf 96 well plate and allow them to come to room temp before using. (return stock to fridge directly and dispose of the unused portion in trough).

8. Obtain the remaining materials and set up the Bravo deck.

9. Transfer 50 μL DNA to be purified in a 96-well Eppendorf plate in a staggered fashion according.

10. Ensure that the water reservoir connected to the pump is full.

11. The program will run the wash station first. Abort the protocol if the water is not flowing. Restart the program until the wash is functioning.

FOLLOWING STEPS ARE AUTOMATED ON BRAVO

12. 65 μL of SPRI beads are added to each well.

13. Pipette mix 25× and incubate for 10 minutes.

14. Separate supernatant from beads by placing plate on Dynal magnet for 6 minutes.

15. Transfer supernatant into a new 96 well Eppendorf plate (located at position 2); discard beads.

16. Add another 120 μL of SPRI beads to each well of the new plate containing the transferred supernatant.

17. Pipette mix 25× and incubate for 10 minutes.

18. Separate supernatant from beads by placing Eppendorf plate on dynal magnet for 6 mins; discard supernatant.

19. Leave plate on magnet and wash beads with 60 μL of 70% Ethanol.

20. Move Eppendorf plate off magnet and air dry SPRI beads for 6 minutes.

21. Add 40 μL of Tris-HCl to each well.

22. Pipette mix 5× and incubate for 3 minutes.

23. Separate liquid from beads on magnet for 6 minutes.

24. Transfer 40 μL eluate into destination 96 well Eppendorf plate.

1.7 Adapter Ligation

1. Starting material is 40 ul elutions from Double sided SPRI cleanup in 96 well plate.

2. Allow 2XLIGBUF600 and IIlumina Oligo tubes to thaw at room temperature.

3. QUICKLIG120 tube should remain in a bench top cooler.

4. Prepare Master mix for 7 or 24 reactions in a 1.5 ml tube as shown in Table below.

| | | For 7 Rxn Mix | | For 24 Rxn | |
|---|---|---|---|---|---|
| Reagent | Tube Name | (ul) | | Mix (ul) | |
| 2X DNA Ligase | 2XLIGBUF600 | 175 | ul | 750 | ul |
| Buffer | | | | | |
| Adapter Oligo Mix | Illumina Oligo | 42 | ul | 180 | ul |
| DNA Ligase (1 U/ul) | QUICKLIG120 | 35 | ul | 150 | ul |
| Nuclease Free Water | NW | 28 | ul | 120 | ul |
| Mix Total Volume | | 280 | | 1200 | |

5. Pipette mix the master mix.

6. Add 20 ul of master mix to each of the samples in the 96 well plate for a total volume of 60 ul for each sample, pipette mixing each sample as the master mix is added.

7. Cap the plate.

8. Transfer to thermocycler and run Adapter Ligation protocol (25° C. for 15 min) for a plate.

9. Proceed to Single SPRI cleanup.

1.8 SPRI Clean Up

See step 1.4 above

2. Bait Preparation

Biotinylated RNA baits are prepared as described in examples 1 and 2 except that MEGAshortscript™ High Yield T7 Transcription Kit from Ambion is used for in vitro transcription instead of the MAXI T7 transcription kit (also from Ambion).

### 3. Hybrid Selection and Capture

3.1 Pond Amplification (“PCR Enrichment”)

1. Starting material is 40 ul of DNA from an Automated SPRI LC protocol before amplification is performed.

2. Place Pfu Ultra II Fusion tubes, DNA samples, tubes dNTPs (25 mMeach) plates, and 15 ml tube in bucket with ice.

3. Each sample requires 20 50 ul PCR reactions.

4. Make a master mix for the appropriate number of samples according to Table below. Vortex gently (speed 6) for 30 sec to mix.

| | For 1 | |
|---|---|---|
| Reagent | Sample (ul) | For 48 Samples (ul) |
| Ultrapure Water | 790 | 49375 |
| PCR primer PE 1.0 | 20 | 1250 |
| PCR primer PE 2.0 | 20 | 1250 |
| 100 mM dNTP mix (25 mM | 10 | 625 |
| each) | | |
| 10 × Pfu Ultra Buffer | 100 | 6250 |
| Pfu Ultra II Fusion HS DNA | 20 | 1250 |
| Polymerase | | |
| Total Master Mix Volume | 960 | 60000 |

5. Pipette 48 ul of master mix into 20 individual wells of a Eppendorf twin tec 96 well plate (for greater than 4 samples multiple 96 well plates will be needed; for a large number of samples automated plate set-up should be used).

6. Transfer 2 ul of sample to each of its 20 corresponding wells (40 ul of total sample will be used).

7. Mix by pipetting 10× and cap plates with strip caps.

8. Transfer to a thermocycler & run an 10 to 12 cycle (based on the test PCR results) Pfu enrichment program (Pond Enrichment 10×/12×(Pfu)); 30 sec at 95° C.; 10-12 cycles of (30 sec at 95° C., 30 sec at 65° C., 30 sec at 72° C.); 10 min at 72° C.; Hold at 4° C.

9. After cycling, pool the 20 reactions for each sample. Proceed to QIA96 plate cleanup, PICO, and Normalization.

3.2 Purification & Concentration (Qiaquick Plate)

See step 1.2 above. Run standard pico green DNA quantitation assay.

3.3 Automated Normalization

1. Open the “Normalization.tab” file within Normalization folder in the A.B. folder on the desktop.

2. Open the “Normalization.tab” file within Normalization folder in the A.B. folder on the desktop.

3. Close this file, when prompted to save choose Save As, delete the quotation marks (“ ”) from either side of the file name (“Normalization.tab” should be Normalization.tab) and save. When prompted to replace the existing file with an error choose “yes” for both.

4. Open the WinPrep application and the Normalization protocol within it.

5. On the Janus, place the uncapped and labeled matrix tubes into the lower right position and a tip box cover reservoir with EB Buffer (Tris-HCl pH 8.0) in the position directly above that.

6. On the left hand side of the program double click on “Custom_{—}1”. Click “Runtime Parameters” on the menu that appears and check that the volumes of EB match those that were entered. Close the menu window.

7. Click “Execute Test” from the top of the program. During the wash steps that follow make sure that there are no air bubbles in the lines. If there are still air bubbles when the machine begins the procedure, click “Pause”, then “Abort” and then “Execute Test” again until the lines are free of bubbles.

8. When the protocol is complete, remove the matrix tube rack and replace it in its position on the Bravo from the Pico protocol.

9. Open the Normalization Protocol and click start. Click “Finish” in the dialog box that appears.

10. When prompted, recap the normalized tubes and uncap the enriched tubes, replacing them in their proper spot on the deck before clicking “Continue”.

11. When the protocol is complete, recap the enriched tubes and clean off the Bravo deck.

3.4 Hybridization

1. Set heat block temperature to 65° C.

2. Prepare HYB buffer in 1.5 mL tube and place in 65° C. heat block.

3. Set 2 thermocyclers to idle at 65° C.

4. Place appropriate baits, DNA samples and PCR plates on ice.

5. Thoroughly clean work surfaces and all objects (pipettes, tip boxes, etc.) that may be used or touched in the process with an RNase wipe.

6. Pipette 2.5 ul of Cot-1 and 2.5 ul Salmon sperm DNA per sample being hybridized into each sample well of a PCR plate.

7. Add 5 ul (100 ng/ul for a total of 500 ng) of a pond sample into each well containing Cot-1 and Salmon sperm and cap the plate with strip caps.

8. On a separate PCR plate, pipette 20 ul HYB buffer into each well that corresponds to a sample well on the sample plate.

9. Cap the HYB buffer plate and place it on one of the thermocyclers set to idle at 65° C. and close the lid.

10. Add 9 ul SUPERase to each tube of bait being used.

11. Pipette 6 ul of appropriate bait into a third PCR plate following the same well pattern as the other two plates and cap it.

12. Place the sample PCR plate onto the unheated thermocycler and begin the hybridization protocol (95° C. 5 minutes then 65° C. forever).

13. After 2 and ½ minutes have passed in the sample plate program, place the bait PCR plate onto the third thermocycler which has been set 65° C. and close the lid.

14. Once the sample plate has been at 95° C. for 5 minutes, pause the program before it starts to ramp down to 65° C. and open the lids of the sample thermocycler and the bait thermocycler.

15. Multichannel pipette 6 ul of each bait into the appropriate sample well.

16. Open the lid of the HYB buffer thermocycler and multichannel pipette 16 ul of HYB buffer into all the corresponding wells of the sample plate.

17. Cap the sample plate and resume the thermocycler program for 65-70 hours.

3.5 Automated Capture

1. Aliquot 180 uL of AP buffer per sample well into a Twin Tec 96 well plate (see Appendix 1).

2. Aliquot 2 mL GS Buffer per sample well into a Costar 96-Deep well plate (see Appendix 1).

3. Aliquot 180 uL of washed Dynabeads M-280 Streptavidin per sample well into a Twin Tec 96 well plate (see Appendix 1 for proper bead preparation).

4. Aliquot 75 uL 0.1N NaOH into a Twin Tec 96 well plate.

5. Aliquot 50 uL 1M Tris-HCL into a Twin Tec 96 well plate (store at 4° C. until used).

6. Place tips into tip box corresponding to sample location in “Hybridization Plate”.

7. Turn on the 2 heating blocks to 78° C. (power sources to the left of the Bravo).

8. Obtain the necessary materials to set up the Bravo deck (see FIG. 12). Start protocol with M-280 Streptavidin beads in position 9, switch plate to 1M Tris-HCl when prompted (after GS washes).

9. Check the water and waste reservoirs below the bench. Make sure the water reservoir is full and the waste reservoir is empty.

10. From the computer desktop, open the software VWorks.

11. Login to the software.

12. Open the protocol: “Capture_{—}6_GS_Washes_Tip_Wash” in the Hybrid Selection folder.

13. Immediately run the Bravo script after placing the reagents into correct positions (Press the start icon and follow the prompts).

14. The first step of the program will run the wash station. Abort the protocol if the water is not flowing, restart the program until the wash is functioning.

15. The program will run for approximately 3 hours until you must intervene. You should replace the tip box in position 3 with a fresh tip box and also replace the M-280 Streptavidin bead plate with a Twin Tec PCR 96 well plate containing 50 uL of 1M Tris-HCl in position 9.

16. At the end of the program, your samples will be located in the 1M Tris-HCl plate at a final volume of 100 uL. Proceed to Cleanup using Qiaquick 96-plate.

17. Dispose of all plates and tips remaining on the Bravo into the biohazard waste. Wipe down the Bravo using 70% EtOH.

3.6 Appendix 1

Buffer Preparation

Make the necessary buffers according to the following formulations:

BW Buffer

40 mL nuclease-free water

10 mL 5M NaCl

50 uL 1M Tris-HCl

100 uL 0.5M EDTA

AP Buffer (Low Stringency Wash)

40 mL nuclease-free water

2.5 mL 20×SSC

500 uL 10% SDS

GS Buffer (High Stringency Wash; Store at 65° C.)

49 mL nuclease-free water

250 uL 20×SSC

500 uL 10% SDS

Bead Preparation

1. Resuspend the Dynabeads M-280 Streptavidin by shaking the vial to obtain a homogeneous suspension

2. For every sample to be captured, transfer 50 uL of Dynabeads to a 15 mL conical tube (it helps to aliquot addition samples worth of beads for dead volume and pipeting error).

3. Wash beads with 200 uL BW buffer per 50 uL beads (i.e. for 200 uL beads use 800 uL of BW buffer)

4. Gently vortex and place tube in DynaMag-15 bead separator for 1-2 minutes. Remove the supernatant without disturbing the attached beads

5. Repeat wash 2×

6. Resuspend beads in 165 uL BW buffer per 50 uL beads

7. Aliquot 180 uL of washed beads into each well of a Twin Tec PCR 96 well plate (beads location should match that of the sample location in the Hybridization plate).

8. Store plate at 4° C. until use.

3.7. Catch Amplification (“Enrichment” PCR)

1. Starting material is 30 ul of DNA from post Capture Qia96 clean up.

2. Place Pfu Ultra II Fusion tubes, DNA samples, tubes dNTPs (25 mM each) plates, and 15 ml tube in bucket with ice.

3. Each sample requires 10 50 ul PCR reactions.

4. Make a master mix for the appropriate number of samples according to Table below. Vortex gently (speed 6) for 30 sec to mix.

| | For 1 | |
|---|---|---|
| Reagent | Sample (ul) | For 48 Samples (ul) |
| Ultrapure Water | 385 | 24062.5 |
| PCR primer PE 1.0 | 10 | 625 |
| PCR primer PE 2.0 | 10 | 625 |
| 100 mM dNTP mix (25 mM | 5 | 312.5 |
| each) | | |
| 10 × Pfu Ultra Buffer | 50 | 3125 |
| Pfu Ultra II Fusion HS | 10 | 24062.5 |
| DNA Polymerase | | |
| Total Master Mix Volume | 470 | 29375 |

5. Pipette 47 ul of master mix into 10 individual wells of a Eppendorf twin tec 96 well plate (for greater than 4 samples multiple 96 well plates will be needed; for a large number of samples automated plate set-up should be used).

6. Transfer 3 ul of sample to each of its 10 corresponding wells (30 ul of total sample will be used).

7. Mix by pipetting 10× and cap plates with strip caps.

8. Transfer to a thermocycler & run an 16 to 20 cycle (based on the test PCR results) Pfu enrichment program (Pond Enrichment 16×/18×/20×(Pfu)); 30 sec at 95° C.; 16-20 cycles of (30 sec at 95° C., 30 sec at 65° C., 30 sec at 72° C.); 10 min at 72° C.; Hold at 4° C.

9. After cycling, pool the 10 reactions for each sample. Proceed to QIA96 plate cleanup, PICO, and Normalization.

3.8 Purification & Concentration (Qiaquick Plate)

See step 1.2 above. Run standard pico green DNA quantitation assay.

3.9 AUTOMATED NORMALIZATION

See step 3.3 above

### 4. Illumina Paired-End Sequencing (Standard Illumina Protocol; not Shown)

### Example 4

### Hybrid Capture from Unamplified Whole-Genome Fragment “Pond” Libraries without Explicit Size Selection

This example describes a method for solution hybrid selection whereby the whole-genome fragment library (“pond”) is neither subjected to an explicit size-selection step (e.g. on an agarose gel) nor PCR-amplified prior to the solution hybridization. The post hybrid-selection PCR amplifications are performed using exemplary conditions that minimize the amplification bias against high GC sequences.

1. Shear 3 μg of human genomic DNA for 4 min. on a Covaris E210 instrument set to duty cycle 5, intensity 5 and 200 cycles per burst such that the mode of the resulting fragment-size distribution (as assayed on an Agilent BioAnalyzer) is ˜250 bp.

2. Clean-up and concentrate sheared DNA using a QIAquick MinElute kit and elute the DNA in 26 μl.

3. Run 1 μl on an Agilent Bioanalyzer to check size distribution.

4. Perform end-repair, phosphorylation and A-addition reactions on the remaining 25 μl as described in the Illumina genomic DNA sample preparation kit and elute the DNA from the QIAquick MinElute in 10 μl EB.

5. Add 6 μl annealed Illumina paired-end adapter oligonucleotides (15 μM), 2 μl 10×T4 DNA ligation buffer (New England Biolabs), 2 μl T4 DNA ligase (NEB #M0202T) and incubate overnight at 16° C.

6. Clean-up and concentrate sheared DNA using a QIAquick MinElute kit and elute the DNA in 10 μl EB. Use a vacuum microcentrifuge to reduce the volume to 5 μl.

7. Prepare 500 ng biotinylated RNA baits from pools of synthetic oligodeoxynucleotides as described in examples 1-3 above. Alternatively, prepare biotinylated RNA transcripts from a concentration-normalized pool of ˜100-300-bp PCR-products amplified with target-specific PCR primer pairs out of total human DNA, whereby one primer of each primer pair has a T7 promoter at the 5′ end, followed by in vitro transcription with a standard Ambion MEGAshortscript™ High Yield T7 Transcription Kit in the presence of biotin UTP and/or biotin CTP).

8. Mix 2 μl of this unamplified “pond” library with 2.5 μl human Cot-1 DNA (1 μg/μl; Invitrogen) and 2.5 μl sonicated fish sperm DNA (1 μg/μl) and set up a solution hybridization with 500 ng biotinylated RNA bait including the bead capture and washing steps as described in example 1 above. Neutralize the 0.1 N NaOH eluate (50 μl) from the beads with 70 μl 1 M Tris-HCl. pH 7.5, desalt and concentrate using the QIAquick Minelute kit eluting the DNA in 10 μl EB.

9. PCR amplify 8 μl of the eluate in a 200 μl PCR reaction (split into 4×50 μl) containing 4 μl each of Illumina PE 1.0 and PE 2.0 PCR primers, 50 μl 5M betaine (Sigma) and 100 μl 2× Phusion HF mastermix with GC buffer (NEB). Thermocycle as follows: 1 min 98° C.; 12-18 Cycles [20 s/98° C., 30 s/65° C., 30 s/72° C.]; 7 m/72° C., ∞/4° C. Alternatively, omit the betaine from the reaction and use a modified thermoprofile: 3 min 98° C.; 12-18 Cycles [60 s/98° C., 30 s/65° C., 30 s/72° C.]; 7 m/72° C., ∞/4° C. Both PCR reaction conditions are designed to minimize the amplification bias against high-GC target sequences.

10. Clean up the reaction using a QIAquick PCR Purification Kit, or, alternatively, a standard Agencourt AmpPure SPRI clean-up kit to remove PCR primers.

11. Perform standard Illumina sequencing library quantitation steps, paired-end cluster-amplification and sequencing reactions to generate 2×76 bases of sequences from each end of the captured genome fragments.

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety for the purposes indicated herein.

We claim:

1. A method for solution-based selection of nucleic acids comprising
hybridizing in solution (1) a group of nucleic acids and (2) a set of bait sequences, to form a hybridization mixture,
contacting the hybridization mixture with a molecule or particle that binds to or is capable of separating the set of bait sequences from the hybridization mixture, and
separating the set of bait sequences from the hybridization mixture to isolate a subgroup of nucleic acids that hybridize to the bait sequences from the group of nucleic acids, wherein the subgroup of nucleic acids is a part or all of a set of target sequences that is desired to be selected,
wherein the sequence composition of the set of bait sequences determines the nucleic acids directly selected from the group of nucleic acids, and wherein the bait sequences are oligonucleotides between about 70 nucleotides and 1000 nucleotides in length.

2. The method of claim 1, wherein the set of bait sequences comprises an affinity tag on each bait sequence.

3. The method of claim 2, wherein the affinity tag is a biotin molecule or a hapten.

4. The method of claim 1, wherein the molecule or particle that binds to or is capable of separating the set of bait sequences from the hybridization mixture binds to the affinity tag.

5. The method of claim 4, wherein the molecule or particle that binds to or is capable of separating the set of bait sequences is an avidin molecule, or an antibody that binds to the hapten or an antigen-binding fragment thereof.

6.-8. (canceled)

9. The method of claim 1, wherein the bait sequences are oligonucleotides between about 100 nucleotides and 300 nucleotides in length.

10. The method of claim 9, wherein the bait sequences are oligonucleotides between about 130 nucleotides and 230 nucleotides in length.

11. The method of claim 10, wherein the bait sequences are oligonucleotides of about 150-200 nucleotides in length.

12. The method of claim 1, wherein the bait sequences are oligonucleotides between about 300 nucleotides and 1000 nucleotides in length.

13. (canceled)

14. (canceled)

15. The method of claim 1, wherein the target-specific sequences in the oligonucleotides are between about 100 and 200 nucleotides in length.

16. The method of claim 15, wherein the target-specific sequences in the oligonucleotides are between about 120 and 170 nucleotides in length.

17.-24. (canceled)

25. The method of claim 1, wherein the bait sequences in the set of bait sequences are RNA molecules.

26. (canceled)

27. The method of claim 1, wherein the group of nucleic acids is fragmented genomic DNA.

28. (canceled)

29. (canceled)

30. The method of claim 1, wherein the target sequences or subgroup of nucleic acids comprises substantially all exons in a genome, or exons from selected genes of interest.

31.-49. (canceled)

50. The method of claim 1, wherein the group of nucleic acids comprises less than 5 microgram of nucleic acids.

51. (canceled)

52. The method of claim 1, wherein the number of bait sequences in the set of bait sequences is greater than 1,000.

53.-57. (canceled)

58. The method of claim 1, wherein the group of nucleic acids is fragmented by physical or enzymatic methods and ligated to synthetic adapters.

59. (canceled)

60. (canceled)

61. The method of claim 1, wherein the selected subgroup of nucleic acids is amplified before being analyzed by sequencing or other methods.

62.-76. (canceled)

77. A method of sequencing or resequencing nucleic acids comprising,
isolating by solution hybridization a subgroup of nucleic acids according to claim 1, and
subjecting the isolated subgroup of nucleic acids to nucleic acid sequencing.

78.-101. (canceled)

102. A method for determining the presence or sequence of a nucleic acid sequence, cell, tissue or organism in a sample, comprising
obtaining a sample containing nucleic acids,
subjecting the nucleic acids in the sample to solution-based selection of nucleic acids according to claim 1, and
determining the presence or sequence of one or more nucleic acids of the subgroup of nucleic acids obtained by selection, whereby the presence or sequence of the one or more nucleic acids indicates the presence of a nucleic acid sequence, cell, tissue or organism in the sample.

103.-109. (canceled)
